SEQUENCE LISTING (61 sequences)

SEQ ID NO: 1 - protein, 365 aa, HOMO SAPIENS
MAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 2 - DNA, 4428 bp, HOMO SAPIENS
ttaaggaaatccgggctgctcttccccatctggaagtggctttccccacatcggctcgtaaactgattatgaaacatacgatgttaattcggagctgcatttcccagctgggcactctcgcgcgctggtccccggggcctcgccccccaccccctgcccttccctcccgcgtcctgcccccatcctccaccccccgcgctggccaccccgcctccttggcagcctctggcggcagcgcgctccactcgcctcccgtgctcctctcgcccatggaattaattctggctccacttgttgctcggcccaggttggggagaggacggagggtggccgcagcgggttcctgagtgaattacccaggagggactgagcacagcaccaactagagaggggtcagggggtgcgggactcgagcgagcaggaaggaggcagcgcctggcaccagggctttgactcaacagaattgagacacgtttgtaatcgctggcgtgccccgcgcacaggatcccagcgaaaatcagatttcctggtgaggttgcgtgggtggattaatttggaaaaagaaactgcctatatcttgccatcaaaaaactcacggaggagaagcgcagtcaatcaacagtaaacttaagagacccccgatgctcccctggtttaacttgtatgcttgaaaattatctgagagggaataaacatcttttccttcttccctctccagaagtccattggaatattaagcccaggagttgctttggggatggctggaagtgcaatgtcttccaagttcttcctagtggctttggccatatttttctccttcgcccaggttgtaattgaagccaattcttggtggtcgctaggtatgaataaccctgttcagatgtcagaagtatatattataggagcacagcctctctgcagccaactggcaggactttctcaaggacagaagaaactgtgccacttgtatcaggaccacatgcagtacatcggagaaggcgcgaagacaggcatcaaagaatgccagtatcaattccgacatcgacggtggaactgcagcactgtggataacacctctgtttttggcagggtgatgcagataggcagccgcgagacggccttcacatacgccgtgagcgcagcaggggtggtgaacgccatgagccgggcgtgccgcgagggcgagctgtccacctgcggctgcagccgcgccgcgcgccccaaggacctgccgcgggactggctctggggcggctgcggcgacaacatcgactatggctaccgctttgccaaggagttcgtggacgcccgcgagcgggagcgcatccacgccaagggctcctacgagagtgctcgcatcctcatgaacctgcacaacaacgaggccggccgcaggacggtgtacaacctggctgatgtggcctgcaagtgccatggggtgtccggctcatgtagcctgaagacatgctggctgcagctggcagacttccgcaaggtgggtgatgccctgaaggagaagtacgacagcgcggcggccatgcggctcaacagccggggcaagttggtacaggtcaacagccgcttcaactcgcccaccacacaagacctggtctacatcgaccccagccctgactactgcgtgcgcaatgagagcaccggctcgctgggcacgcagggccgcctgtgcaacaagacgtcggagggcatggatggctgcgagctcatgtgctgcggccgtgggtacgaccagttcaagaccgtgcagacggagcgctgccactgcaagttccactggtgctgctacgtcaagtgcaagaagtgcacggagatcgtggaccagtttgtgtgcaagtagtgggtgccacccagcactcagccccgctcccaggacccgcttatttatagaaagtacagtgattctggtttttggtttttagaaatattttttatttttccccaagaattgcaaccggaaccattttttttcctgttaccatctaagaactctgtggtttattattaatattataattattatttggcaataatgggggtgggaaccacgaaaaatatttattttgtggatctttgaaaaggtaatacaagacttcttttggatagtatagaatgaagggggaaataacacataccctaacttagctgtgtgggacatggtacacatccagaaggtaaagaaatacattttctttttctcaaatatgccatcatatgggatgggtaggttccagttgaaagagggtggtagaaatctattcacaattcagcttctatgaccaaaatgagttgtaaattctctggtgcaagataaaaggtcttgggaaaacaaaacaaaacaaaacaaacctcccttccccagcagggctgctagcttgctttctgcattttcaaaatgataatttacaatggaaggacaagaatgtcatattctcaaggaaaaaaggtatatcacatgtctcattctcctcaaatattccatttgcagacagaccgtcatattctaatagctcatgaaatttgggcagcagggaggaaagtccccagaaattaaaaaatttaaaactcttatgtcaagatgttgatttgaagctgttataagaattgggattccagatttgtaaaaagacccccaatgattctggacactagattttttgtttggggaggttggcttgaacataaatgaaatatcctgtattttcttagggatacttggttagtaaattataatagtagaaataatacatgaatcccattcacaggtttctcagcccaagcaacaaggtaattgcgtgccattcagcactgcaccagagcagacaacctatttgaggaaaaacagtgaaatccaccttcctcttcacactgagccctctctgattcctccgtgttgtgatgtgatgctggccacgtttccaaacggcagctccactgggtcccctttggttgtaggacaggaaatgaaacattaggagctctgcttggaaaacagttcactacttagggatttttgtttcctaaaacttttattttgaggagcagtagttttctatgttttaatgacagaacttggctaatggaattcacagaggtgttgcagcgtatcactgttatgatcctgtgtttagattatccactcatgcttctcctattgtactgcaggtgtaccttaaaactgttcccagtgtacttgaacagttgcatttataaggggggaaatgtggtttaatggtgcctgatatctcaaagtcttttgtacataacatatatatatatatacatatatataaatataaatataaatatatctcattgcagccagtgatttagatttacagcttactctggggttatctctctgtctagagcattgttgtccttcactgcagtccagttgggattattccaaaagttttttgagtcttgagcttgggctgtggccccgctgtgatcataccctgagcacgacgaagcaacctcgtttctgaggaagaagcttgagttctgactcactgaaatgcgtgttgggttgaagatatctttttttcttttctgcctcacccctttgtctccaacctccatttctgttcactttgtggagagggcattacttgttcgttatagacatggacgttaagagatattcaaaactcagaagcatcagcaatgtttctcttttcttagttcattctgcagaatggaaacccatgcctattagaaatgacagtacttattaattgagtccctaaggaatattcagcccactacatagatagctttttttttttttttttttttttaataaggacacctctttccaaacaggccatcaaatatgttcttatctcagacttacgttgttttaaaagtttggaaagatacacatcttttcatacccccccttaggaggttgggctttcatatcacctcagccaactgtggctcttaatttattgcataatgatatccacatcagccaactgtggctctttaatttattgcataatgatattcacatcccctcagttgcagtgaattgtgagcaaaagatcttgaaagcaaaaagcactaattagtttaaaatgtcacttttttggtttttattatacaaaaaccatgaagtactttttttatttgctaaatcagattgttcctttttagtgactcatgtttatgaagagagttgagtttaacaatcctagcttttaaaagaaactatttaatgtaaaatattctacatgtcattcagatattatgtatatcttctagcctttattctgtacttttaatgtacatatttctgtcttgcgtgatttgtatatttcactggtttaaaaaacaaacatcgaaaggcttattccaaatggaagatagaatataaaataaaacgttacttgtaaaaaaaaaaaa

SEQ ID NO: 3 - DNA, 2460 bp, HOMO SAPIENS
cgtggcacgcgcggaagattctcagtgtccttacagagtcatcttccctgagccccggaagtgttggaaaacatttagccccttctttgggaaactcagtttctgatcagaatttttgttttaccctggggttgacagtctcgccagaggtctcatttcatactgtcttttcggatctgatcctcttggtaaacaggcggggatgttttaccctacagagccgatgtatgtgtgagttcgctgtgagttctttgagtgtctcaaacttgtggggccttttctcggttgcactgggattgaagagggaagaggcccaaggtgtttccgggcaagcggcggggttaagtggagatgcgactcgtgaggctctcctttccgatccccctttgggacaccctctgcctacctctaccctggagccagggagacccaagtcttggtgaccggatgggcccgctctcagttggcctgggctctgggaactggtggactctccctgggggcttcgggctgggagtgggttcggtttgtgtggcttcggctctaacaaagagatccgctgtaatccgccgaatctgttatcaatttctctgctgcttgagccccgccccacgcgccccgcccgccgcgaagcttggaaagtgcacgcggccagcaccaatctgggccgctgactcggaaacatgtcgcagcgtgtgtgtctatggacgcgtgtgagtgtgtaaatgtgcacgagtgtgaatgtgtatgatgtgtgtgcacgcggcatcggctgcccttggggagagttgactttgcagcctgggctgcgcgagaagcagactttgcagcccactccctcccctggaggaaatttgacacttagggcgggggtggggagatagccggagccttctctctcctagctggggaaaccccagatttccattctccaggatgcgccccccagctttgcagcgtcttggggacaactggcctggtgttggagccctgcttagcaggcgctggggaccacataagcattcctctttggagaagccccgaagcgtccaggccaaagggggcggttcacggaagaaaaaccttgcacgcccttgagcgcatagctttaccagggctgcctaggtcccgcctcttgcccttttacggcacaggttccaagccaggctcttcccaccgccttaaagaggctcacctttcttttcttttctgtggaaggggctccttcaggggctatgggcgatgcagtgcggcagggttagacttacgtgtaaggggatttttaaaacccgctcctcccacccgcacccgccacctactcgctccgccgccgcctacaggtggagaagtcaccagtggggaggaacggcagcggaagcttccaaggccaactcctacccctgaaattcttcaggaagggaaccttcgccgctggggggctctttggcctggaatcgatgcgcccagctgcggctcggaagccagcgcctctggccccgtctggactcatctgcaagggctctggcctcgccccgcacccccacctttcgggactgaccgaaccaagtctgagttgggctggagaggctagactggaggcagggtggcagagttccaacgacaggctcgcagtgccgcgaatggcaaagtgggccacaaccccagatcaggacccagagaaactggagtctctctctgggcctcccatctcctccctccctggcaactaccaggttgtggggtgggagggagagtgaaaaatcaagaatttgggagaaagctgtggggagggcagggaagggatccttctccccggggaagcgagacccagactcccttctttcctctagggttccatcccttctctcagtccgtggaagaggccacaggcgacgcgggcgagggtggcactcttttccagtttccttggttgggagacccgacctctctctccattatcccctagggcccccatctccttctcccctccctagtctggctgaagaacgtccttaaggaaatccgggctgctcttccccatctggaagtggctttccccacatcggctcgtaaactgattatgaaacatacgatgttaattcggagctgcatttcccagctgggcactctcgcgcgctggtccccggggcctcgccccccaccccctgcccttccctcccgcgtcctgcccccatcctccaccccccgcgctggccaccccgcctccttggcagcctctggcggcagcgcgctccactcgcctcccgtgctcctctcgcccatggaattaattctggctccacttgttgctcggcccaggttggtgagaggacggagggtgcccacagcgggttcctgagtgaattacccaggagggactgagcacagcaccaactagaggggggccagggggtgcgggactcgagcgagcaggaaggaggcagcgcctggcaccagggc

SEQ ID NO: 4 - protein, 585 aa, HOMO SAPIENS
MARPDPSAPPSLLLLLLAQLVGRAAAASKAPVCQEITVPMCRGIGYNLTHMPNQFNHDTQDEAGLEVHQFWPLVEIQCSPDLRFFLCTMYTPICLPDYHKPLPPCRSVCERAKAGCSPLMRQYGFAWPERMSCDRLPVLGRDAEVLCMDYNRSEATTAPPRPFPAKPTLPGPPGAPASGGECPAGGPFVCKCREPFVPILKESHPLYNKVRTGQVPNCAVPCYQPSFSADERTFATFWIGLWSVLCFISTSTTVATFLIDMDTFRYPERPIIFLSACYLCVSLGFLVRLVVGHASVACSREHNHIHYETTGPALCTIVFLLVYFFGMASSIWWVILSLTWFLAAAMKWGNEAIAGYGQYFHLAAWLIPSVKSITALALSSVDGDPVAGICYVGNQNLNSLRRFVLGPLVLYLLVGTLFLLAGFVSLFRIRSVIKQGGTKTDKLEKLMIRIGIFTLLYTVPASIVVACYLYEQHYRESWEAALTCACPGHDTGQPRAKPEYWVLMLKYFMCLVVGITSGVWIWSGKTVESWRRFTSRCCCRPRRGHKSGGAMAAGDYPEASAALTGRTGPPGPAATYHKQVSLSHV

SEQ ID NO: 5 - DNA, 2334 bp, HOMO SAPIENS
acccagggacggaggacccaggctggcttggggactgtctgctcttctcggcgggagccgtggagagtcctttccctggaatccgagccctaaccgtctctccccagccctatccggcgaggagcggagcgctgccagcggaggcagcgccttcccgaagcagtttatctttggacggttttctttaaaggaaaaacgaaccaacaggttgccagccccggcgccacacacgagacgccggagggagaagccccggcccggattcctctgcctgtgtgcgtccctcgcgggctgctggaggcgaggggagggagggggcgatggctcggcctgacccatccgcgccgccctcgctgttgctgctgctcctggcgcagctggtgggccgggcggccgccgcgtccaaggccccggtgtgccaggaaatcacggtgcccatgtgccgcggcatcggctacaacctgacgcacatgcccaaccagttcaaccacgacacgcaggacgaggcgggcctggaggtgcaccagttctggccgctggtggagatccaatgctcgccggacctgcgcttcttcctatgcactatgtacacgcccatctgtctgcccgactaccacaagccgctgccgccctgccgctcggtgtgcgagcgcgccaaggccggctgctcgccgctgatgcgccagtacggcttcgcctggcccgagcgcatgagctgcgaccgcctcccggtgctgggccgcgacgccgaggtcctctgcatggattacaaccgcagcgaggccaccacggcgccccccaggcctttcccagccaagcccacccttccaggcccgccaggggcgccggcctcggggggcgaatgccccgctgggggcccgttcgtgtgcaagtgtcgcgagcccttcgtgcccattctgaaggagtcacacccgctctacaacaaggtgcggacgggccaggtgcccaactgcgcggtaccctgctaccagccgtccttcagtgccgacgagcgcacgttcgccaccttctggataggcctgtggtcggtgctgtgcttcatctccacgtccaccacagtggccaccttcctcatcgacatggacacgttccgctatcctgagcgccccatcatcttcctgtcagcctgctacctgtgcgtgtcgctgggcttcctggtgcgtctggtcgtgggccatgccagcgtggcctgcagccgcgagcacaaccacatccactacgagaccacgggccctgcactgtgcaccatcgtcttcctcctggtctacttcttcggcatggccagctccatctggtgggtcatcctgtcgctcacctggttcctggccgccgcgatgaagtggggcaacgaggccatcgcgggctacggccagtacttccacctggctgcgtggctcatccccagcgtcaagtccatcacggcactggcgctgagctccgtggacggggacccagtggccggcatctgctacgtgggcaaccagaacctgaactcgctgcggcgcttcgtgctgggcccgctggtgctctacctgctggtgggcacgctcttcctgctggcgggcttcgtgtcgctcttccgcatccgcagcgtcatcaagcagggcggcaccaagacggacaagctggagaagctcatgatccgcatcggcatcttcacgctgctctacacggtccccgccagcattgtggtggcctgctacctgtacgagcagcactaccgcgagagctgggaggcggcgctcacctgcgcctgcccgggccacgacaccggccagccgcgcgccaagcccgagtactgggtgctcatgctcaagtacttcatgtgcctggtggtgggcatcacgtcgggcgtctggatctggtcgggcaagacggtggagtcgtggcggcgtttcaccagccgctgctgctgccgcccgcggcgcggccacaagagcgggggcgccatggccgcaggggactaccccgaggcgagcgccgcgctcacaggcaggaccgggccgccgggccccgccgccacctaccacaagcaggtgtccctgtcgcacgtgtaggaggctgccgccgagggactcggccggagagctgaggggaggggggcgttttgtttggtagttttgccaaggtcacttccgtttaccttcatggtgctgttgccccctcccgcggcgacttggagagagggaagaggggcgttttcgaggaagaacctgtcccaggtcttctccaaggggcccagctcacgtgtattctattttgcgtttcttacctgccttctttatgggaaccctctttttaatttatatgtat

SEQ ID NO: 6 - protein, 570 aa, RATTUS SP.
MRARSALPRSALPRLLLPLLLLPAAGPAQFHGEKGISIPDHGFCQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQICVGQNHSEDGTPALLTTAPPSGLQPGAGGTPGGPGGGGAPPRYATLEHPFHCPRVLKVPSYLSYKFLGERDCAAPCEPARPDGSMFFSHHHTRFARLWILTWSVLCCASTFFTVTTSLVAMQRFRYPERPIIFLSGCYTMVSVAYIAGFVLQERVVCNERFSEDGYRTVGQGTKKEGCTILFMMLYFFSMASSIWWVILSLTWFLAAGMKWGHAAIEANSQYFHLAAWAVPAVKTITILAMGQIDGDLLSGVCFVGLNRLDPLRGFVLAPLFVYLFIGTSFLLAGFVSLFRIRTIMKHDGTKTEPLERLMVRIGVFSVLYTVPATIVIACYFYEQAFREHWERSWVSQHCKSLAIPCPAHYTPRTSPDFTVYMIKYLMTLIVGITSGFWIWSGKTLHSWRKFYTRLTNSRHGETTV

SEQ ID NO: 7 - DNA, 1912 bp, RATTUS SP.
aggggaaggcgcgcggtctctgggttgggggcgggggctggggggcgcccaggagccgagtggggggcggcggccagcatgcgggcccgcagcgccctgccccgcagcgccctgccccgcctgctgctgccactgctgctgctgccggctgccgggccggctcagttccacggggagaagggcatctccatcccggaccacggcttctgccagcccatctccatcccgctgtgcacggacatcgcctacaaccagaccatcatgcccaaccttcttgggcacacgaaccaagaggacgcgggcctggaggtgcatcaattctacccgctggtgaaggtgcagtgctcgcccgagctgcgcttcttcctgtgctccatgtacgctccggtgtgcacggtgctggagcaggccatcccgccgtgccgctccatctgcgaacgcgcgcgccaaggctgcgaggcgctcatgaacaagttcggcttccagtggcccgagcgcctccgctgcgagcatttcccgcgtcacggcgcggagcagatctgcgtgggccagaaccactccgaggacggaactcctgcgctactcaccaccgcgccaccgtctgggctgcagcctggcgctggtggcaccccgggcggccctggcggtggtggcgcgcccccgcgctacgccactctggagcaccctttccactgtccccgcgtcctcaaggtgccgtcctatctcagctataagtttctgggtgagcgcgattgtgccgcgccctgcgagcctgcacggcccgacggctccatgttcttctcgcaccaccacactcgttttgcccgtctctggatcctcacatggtcggtgctgtgctgcgcttctactttcttcacggtcaccacctctttagtggccatgcagcgattccgctacccagagcggcccatcatcttcctgtccggttgctacaccatggtgtcagtggcctacattgcgggcttcgtgctccaggagcgcgtggtgtgcaacgagcgcttctctgaggacggttatcgcacggtggggcagggcactaagaaagaaggctgtactatactcttcatgatgctctacttcttcagtatggccagctccatctggtgggtgattctgtccctcacctggttcctggcagccggtatgaagtggggccacgcggccatcgaggccaattcgcagtacttccacctggccgcctgggcggtgccggccgtcaaaaccatcaccatcctggccatgggccagatcgacggcgacctgctgagcggcgtgtgcttcgtgggcctcaacaggctggacccgctgcgaggcttcgtgctggcgccgctcttcgtgtacctgttcatcggcacatccttcctgctggcgggcttcgtgtcactcttccgcatccgcaccatcatgaagcacgacggcaccaagacggagccgctggagaggctcatggtgcgtatcggcgtcttctccgtgctctacaccgtaccggccaccatcgtcatcgcctgctacttctatgagcaggccttccgcgagcactgggagcgctcgtgggtaagccagcactgcaagagcctagccatcccctgcccggcccactacacgccccgcacgtcgcccgacttcacagtctacatgatcaaatacctcatgacgctcatcgtgggcatcacgtcgggcttctggatctggtccggcaagacgctgcactcgtggaggaagttctacacgcgtctcaccaacagccggcatggagagaccaccgtgtgaagcggtctcgctgctgggcgcccccctctcccaggtccggactgcaaccgtgccctccttcactcgggaggggggtgcaccctacggactcctattttatttttttaaataaagaacagtg

SEQ ID NO: 8 - DNA, 27 bp, Artificial Sequence
  primer
aatgtcttccaagttcttcctagtggc

SEQ ID NO: 9 - DNA, 22 bp, Artificial Sequence
  primer
gatgtcggaattgatactggca

SEQ ID NO: 10 - DNA, 20 bp, Artificial Sequence
  primer
accacagtccatgccatcac

SEQ ID NO: 11 - DNA, 20 bp, Artificial Sequence
  primer
tccaccaccctgttgctgta

SEQ ID NO: 12 - DNA, 38 bp, Artificial Sequence
  probe
gtcctgggagcggggcttttttctcttggaaagaaagt

SEQ ID NO: 13 - DNA, 46 bp, Artificial Sequence
  probe
agagttcttagatggtaacaggaaatttttctcttggaaagaaagt

SEQ ID NO: 14 - DNA, 47 bp, Artificial Sequence
  probe
tatttcccccttcattctatactatctttttctcttggaaagaaagt

SEQ ID NO: 15 - DNA, 48 bp, Artificial Sequence
  probe
catagaagctgaattgtgaatagattttttttctcttggaaagaaagt

SEQ ID NO: 16 - DNA, 38 bp, Artificial Sequence
  probe
gccctgctggggaagggtttttctcttggaaagaaagt

SEQ ID NO: 17 - DNA, 40 bp, Artificial Sequence
  probe
cctccctgctgcccaaatttttttctcttggaaagaaagt

SEQ ID NO: 18 - DNA, 44 bp, Artificial Sequence
  probe
atcccaattcttataacagcttctttttctcttggaaagaaagt

SEQ ID NO: 19 - DNA, 43 bp, Artificial Sequence
  probe
cttgttgcttgggctgagaaactttttctcttggaaagaaagt

SEQ ID NO: 20 - DNA, 43 bp, Artificial Sequence
  probe
ggctcagtgtgaagaggaaggttttttctcttggaaagaaagt

SEQ ID NO: 21 - DNA, 40 bp, Artificial Sequence
  probe
ccagtggagctgccgtttgtttttctcttggaaagaaagt

SEQ ID NO: 22 - DNA, 49 bp, Artificial Sequence
  probe
cactgtactttctataaataagcggtttttaggcataggacccgtgtct

SEQ ID NO: 23 - DNA, 48 bp, Artificial Sequence
  probe
tttctaaaaaccaaaaaccagaattttttaggcataggacccgtgtct

SEQ ID NO: 24 - DNA, 43 bp, Artificial Sequence
  probe
aaaaatggttccggttgcatttttaggcataggacccgtgtct

SEQ ID NO: 25 - DNA, 57 bp, Artificial Sequence
  probe
caaataataattataatattaataataaaccactttttaggcataggacccgtgtct

SEQ ID NO: 26 - DNA, 51 bp, Artificial Sequence
  probe
agatccacaaaataaatatttttcgtgtttttaggcataggacccgtgtct

SEQ ID NO: 27 - DNA, 50 bp, Artificial Sequence
  probe
ccacacagctaagttagggtatgtgttttttaggcataggacccgtgtct

SEQ ID NO: 28 - DNA, 49 bp, Artificial Sequence
  probe
ttaccttctggatgtgtaccatgtctttttaggcataggacccgtgtct

SEQ ID NO: 29 - DNA, 47 bp, Artificial Sequence
  probe
aacctacccatcccatatgatggtttttaggcataggacccgtgtct

SEQ ID NO: 30 - DNA, 45 bp, Artificial Sequence
  probe
ctaccaccctctttcaactggtttttaggcataggacccgtgtct

SEQ ID NO: 31 - DNA, 50 bp, Artificial Sequence
  probe
ccagagaatttacaactcattttggttttttaggcataggacccgtgtct

SEQ ID NO: 32 - DNA, 46 bp, Artificial Sequence
  probe
tcccaagaccttttatcttgcatttttaggcataggacccgtgtct

SEQ ID NO: 33 - DNA, 49 bp, Artificial Sequence
  probe
aggtttgttttgttttgttttgttttttttaggcataggacccgtgtct

SEQ ID NO: 34 - DNA, 51 bp, Artificial Sequence
  probe
gtccttccattgtaaattatcattttgtttttaggcataggacccgtgtct

SEQ ID NO: 35 - DNA, 49 bp, Artificial Sequence
  probe
ttttccttgagaatatgacattctttttttaggcataggacccgtgtct

SEQ ID NO: 36 - DNA, 50 bp, Artificial Sequence
  probe
ggagaatgagacatgtgatatacctttttttaggcataggacccgtgtct

SEQ ID NO: 37 - DNA, 48 bp, Artificial Sequence
  probe
tctgtctgcaaatggaatatttgatttttaggcataggacccgtgtct

SEQ ID NO: 38 - DNA, 48 bp, Artificial Sequence
  probe
tcatgagctattagaatatgacggtttttaggcataggacccgtgtct

SEQ ID NO: 39 - DNA, 48 bp, Artificial Sequence
  probe
aattttttaatttctggggacttttttttaggcataggacccgtgtct

SEQ ID NO: 40 - DNA, 52 bp, Artificial Sequence
  probe
aaatcaacatcttgacataagagttttatttttaggcataggacccgtgtct

SEQ ID NO: 41 - DNA, 46 bp, Artificial Sequence
  probe
ggggtctttttacaaatctggatttttaggcataggacccgtgtct

SEQ ID NO: 42 - DNA, 51 bp, Artificial Sequence
  probe
agaaaatacaggatatttcatttatgttttttaggcataggacccgtgtct

SEQ ID NO: 43 - DNA, 56 bp, Artificial Sequence
  probe
tactattataatttactaaccaagtatccctatttttaggcataggacccgtgtct

SEQ ID NO: 44 - DNA, 51 bp, Artificial Sequence
  probe
ctgtgaatgggattcatgtattatttctttttaggcataggacccgtgtct

SEQ ID NO: 45 - DNA, 44 bp, Artificial Sequence
  probe
gctgaatggcacgcaattactttttaggcataggacccgtgtct

SEQ ID NO: 46 - DNA, 46 bp, Artificial Sequence
  probe
aggttgtctgctctggtgcagttttttaggcataggacccgtgtct

SEQ ID NO: 47 - DNA, 47 bp, Artificial Sequence
  probe
cacaacacggaggaatcagagagtttttaggcataggacccgtgtct

SEQ ID NO: 48 - DNA, 45 bp, Artificial Sequence
  probe
gaaacgtggccagcatcacattttttaggcataggacccgtgtct

SEQ ID NO: 49 - DNA, 45 bp, Artificial Sequence
  probe
tgtcctacaaccaaaggggactttttaggcataggacccgtgtct

SEQ ID NO: 50 - DNA, 46 bp, Artificial Sequence
  probe
gagctcctaatgtttcatttcctttttaggcataggacccgtgtct

SEQ ID NO: 51 - DNA, 46 bp, Artificial Sequence
  probe
agttttaggaaacaaaaatccctttttaggcataggacccgtgtct

SEQ ID NO: 52 - DNA, 49 bp, Artificial Sequence
  probe
gaaaactactgctcctcaaaataaatttttaggcataggacccgtgtct

SEQ ID NO: 53 - DNA, 24 bp, Artificial Sequence
  probe
attcttggggaaaaataaaaaata

SEQ ID NO: 54 - DNA, 20 bp, Artificial Sequence
  probe
gttcccacccccattattgc

SEQ ID NO: 55 - DNA, 28 bp, Artificial Sequence
  probe
caaaagaagtcttgtattaccttttcaa

SEQ ID NO: 56 - DNA, 29 bp, Artificial Sequence
  probe
catatttgagaaaaagaaaatgtatttct

SEQ ID NO: 57 - DNA, 23 bp, Artificial Sequence
  probe
aaaatgcagaaagcaagctagca

SEQ ID NO: 58 - DNA, 27 bp, Artificial Sequence
  probe
caaaaaatctagtgtccagaatcattg

SEQ ID NO: 59 - DNA, 19 bp, Artificial Sequence
  probe
tcaagccaacctccccaaa

SEQ ID NO: 60 - DNA, 24 bp, Artificial Sequence
  probe
ggatttcactgtttttcctcaaat

SEQ ID NO: 61 - DNA, 25 bp, Artificial Sequence
  probe
taagtagtgaactgttttccaagca

(19) United States
(12) Patent Application Publication
(10) Pub. No.: US 2005/0182129 A1
(43) Pub. Date: Aug. 18, 2005

(54) Hair growth tonic

(76) Inventors: Akiko Ikeda, Tokyo (JP); Hideki Shinonaga, Tokyo (JP); Natsuko Fujimoto, Tokyo (JP); Yoko Kasai, Tokyo (JP)

Correspondence Address: SUGHRUE MION, PLLC, 2100 PENNSYLVANIA AVENUE, N.W., SUITE 800, WASHINGTON, DC, 20037, US

(21) Appl. No.: 10/511,632

(22) PCT Filed: Apr. 17, 2003
(86) PCT No.: PCT/JP03/04884

(30) Foreign Application Priority Data
Apr. 17, 2002 (JP) 2002-115529

(51) Int. Cl. A61K 31/365; C07D 313/08

(52) U.S. Cl. 514/450; 549/269

(57) ABSTRACT

A dermal papilla cell growth promoter, a hair growth stimulant and a hair growth tonic containing a compound having an activity of inhibiting the function of WNT-5A.

### TECHNICAL FIELD

The present invention relates to a dermal papilla cell growth promoter, a hair growth stimulant and a hair growth tonic. More specifically, it relates to a dermal papilla cell growth promoter, a hair growth stimulant and a hair growth tonic comprising a WNT-5A function inhibitor as an active ingredient. Furthermore, the present invention relates to a method of screening a dermal papilla cell growth promoter based on the effect of inhibiting the function of WNT-5A.

### BACKGROUND ART

Human hair follicles are made up of epithelial and mesenchymal dermal cells such as keratinocytes, dermal papilla cells, fibroblasts and sebaceous cells and hair growth cycle (hair cycle) is controlled via interactions among these cells. The hair shaft is formed by the proliferation/differentiation (keratinization) of follicular keratinocytes. Dermal papillae regulate the proliferation, differentiation and apoptosis of these follicular keratinocytes and thus play a key role in the control of the hair cycle. In developing a hair growth stimulant/a hair growth tonic, therefore, it would be important to study effects on dermal papilla cells. However, molecular mechanisms regulating the proliferating ability and hair-cycle controlling ability of dermal papilla cells have been scarcely clarified so far.

On the other hand, WNT-5A is a secretor glycoprotein belonging to the WNT family. The WNT family includes about 20 kinds of molecules which are conserved over a wide range from nematodes to mammals. It is known that these WNTs are important intercellular signaling molecules which regulate axis formation and organ formation during the fetal stage (Annu. Rev. Cell Dev. Biol, 14, 59-88 (1998); Genes & Dev. 11, 3286-3305 (1997)). In humans, there are 10 kinds of WNT receptors that are Frizzled seven-pass transmembrane receptors (Annu. Rev. Cell Dev. Biol., 14, 59-88 (1998), Genes & Dev., 11, 3286-3305 (1997)). Depending on WNT-Frizzled bond combinations, there exist 3 kinds of signal transduction pathways (i.e., WNT/β-catenin pathway, PCP pathway and WNT/Ca²⁺ pathway) (Annu. Rev. Cell Dev. Biol., 14, 59-88 (1998)).

In recent years, it is clarified that the WNT/β-catenin pathway is fundamental to the formation of hair follicles (Genes & Dev., 8, 2691-2703 (1994); Cell, 95, 605-614 (1998), Dev. Biol., 207, 133-149 (1999); Genes & Dev., 14, 1181-1185 (2000); Cell, 105, 533-545 (2001)). In 1988, a stabilized β-catenin transgenic mouse in the skin was constricted. It is reported that this mouse showed increased de novo follicular morphogenesis and led to hypertrichosis (Cell, 95, 605-614 (1998)). It was reported in 2000 that the WNT/β-catenin signal plays an important role in maintaining the hair-inducing activity of dermal papilla (Genes & Dev., 14, 1181-1185 (2000)).

However, it has been clarified that signal transduction from WNT-5A is mediated not by the WNT/β-catenin pathway but by the Ca²⁺ pathway (Dev. Biol., 182, 114-120 (1997); Curr. Biol., 9, 695-698 (1999)) and nothing has been reported so far with respect to the relationship between WNT-5A and follicle morphogenesis.

It is furthermore reported that duplication of the embryonic axis is induced by injecting Xenopus laevis WNT-5A mRNA together with human Frizzled 5 mRNA into an early of Xenopus laevis embryo (Science, 275, 1652-1654 (1997)) while the axis duplication induced by the injection of WNT-1 or WNT-8 mRNA is inhibited by WNT-5A (J. Cell Biol., 133, 1123-1137 (1996)), and that Xenopus laevis WNT-5A binds to rat Frizzled 2 and activates CamKII (Ca²⁺/calmodulin-dependent protein kinase II) and PKC (protein kinase II) via the Ca²⁺ pathway (Curr. Biol., 9, 695-698 (1999)). However, physiological meanings thereof still remain unknown and nothing has been reported so far with respect to the relationship between WNT-5A and hair growth stimulation/hair growth promotion.

### DISCLOSURE OF THE INVENTION

An object of the present invention is to provide a screening method using a molecule regulating the proliferation of dermal papilla cells, a dermal papilla cell growth promoter and a hair growth stimulant and a hair growth tonic based on a novel function.

In order to achieve these objects, the present inventors conducted intensive studies. As a result, they have found that WNT-5A is highly expressed in dermal papilla cells and WNT-5A relates to the proliferating ability of dermal papilla cells. As the results of subsequent studies based on these findings, they have further found that the growth of dermal papilla cells can be remarkably promoted and the hair bulb part of follicles can be enlarged by inhibiting the function of WNT-5A, thereby completing the present invention.

That is, in one embodiment, the present invention provides a dermal papilla cell growth promoter which comprises a compound having an activity of inhibiting the function of WNT-5A.

In another embodiment, the present invention provides a dermal papilla cell growth promoter which comprises a compound having an activity of inhibiting the production of WNT-5A.

In still another embodiment, the present invention provides a compound represented by the following formula (I): 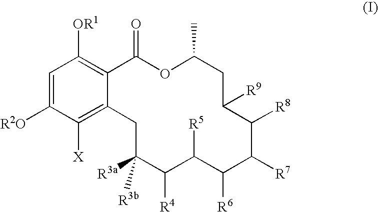
where in R¹ and R² are the same or different and each represents a hydrogen atom, a C₁₋₆ alkyl group or a C₂₋₆ alkanoyl group;
X represents a hydrogen atom or a halogen atom;
R^{3a} and R^{3b} are the same or different and each represents a hydrogen atom or a hydroxyl group; and

R⁴, R⁵, R⁶, R⁷, R⁸ and R⁹ are the same or different and each represents a hydrogen atom, a hydroxyl group, a halogen atom or a C₂₋₆ alkanoyloxy group, or adjacent groups thereof are taken together to form a π bond or an ether bond, or R⁵ and R⁸ or R⁵ and R⁹ are taken together to form an ether bond.

In still another embodiment, the present invention provides a dermal papilla cell growth promoter which comprises a compound represented by the following formula (II): 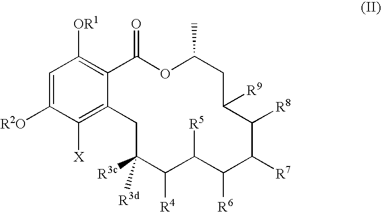
wherein R¹ and R² are the same or different and each represents a hydrogen atom, a C₁₋₆ alkyl group or a C₂₋₆ alkanoyl group;
X represents a hydrogen atom or a halogen atom;
R^{3c} and R^{3d} are the same or different and each represents a hydrogen atom, a hydroxyl group or a C₁₋₆ alkoxy group, or R^{3c} and R^{3d} are taken together to form an oxo group, a hydroxyimino group or a C₁₋₆ alkoxyimino group; and
R⁴, R⁵, R⁶, R⁷, R⁸ and R⁹ are the same or different and each represents a hydrogen atoms a hydroxyl group, a halogen atom or a C₂₋₆ alkanoyloxy group, or adjacent groups thereof are taken together to form a π bond or an ether bond, or R⁵ and R⁸ or R⁵ and R⁹ are taken together to form an ether bond.

In still another embodiment, the present invention provides a dermal papilla cell growth promoter containing a compound represented by the following formula (IX): 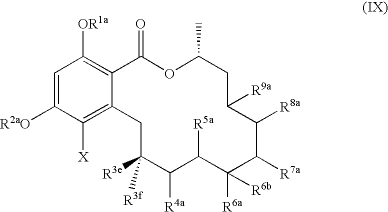
wherein R^{1a} and R^{2a} are the same or different and each represents a hydrogen atom, a C₁₋₆ alkyl group, a C₂₋₆ alkanoyl group,
a group represented by (CH₂)ₚCO—Y—R¹⁰ (wherein Y represents an oxygen atom or a sulfur atom; R¹⁰ represents a hydrogen atom, a C₁₋₆ alkyl group or a substituted or unsubstituted aryl group; and p is 0 or 1),
a group represented by (CH₂)_{q}R¹¹ (wherein R¹¹ represents a substituted or unsubstituted cycloalkyl group or a substituted or unsubstituted C₂₋₁₀ heterocycle; and q is 0 or 1), or
a group represented by COR¹² (wherein R¹² represents a substituted or unsubstituted aryl group or a substituted or unsubstituted C₂₋₁₀ heterocycle);
X represents a hydrogen atom or a halogen atom;
R^{3e} and R^{3f} are the same or different and each represents a hydrogen atom, a hydroxyl group, a C₁₋₆ alkoxy group or a C₁₋₆ alkanoyloxy group, or R^{3e} and R^{3f} are taken together to form an oxo group, a hydroxyimino group or a C₁₋₆ alkoxyimino group; and
R^{4a}, R^{5a}, R^{6a}, R^{7a}, R^{8a} and R^{9a} are the same or different and each represents a hydrogen atom, a hydroxyl group, a halogen atom or
a group represented by Z-R¹³ (wherein Z represents an oxygen atom or a sulfur atom; and R¹³ represents a C₁₋₆ alkyl group, a C₁₋₆ alkanoyl group or a substituted or unsubstituted aryl group), or
adjacent groups thereof are taken together to form a π bond or an ether bond, or
R^{5a} and R^{8a} or R^{5a} and R^{9a} are taken together to form an ether bond; and
R^{6b} represents a hydrogen atom, or is taken together with R^{3e} or R^{3f} to form an ether bond.

In still another embodiment, the present invention provides a hair growth stimulant or a hair growth tonic containing the above-described dermal papilla cell growth promoter as an active ingredient.

In still another embodiment, the present invention provides a method of screening a dermal papilla cell growth promoter, which comprises selecting a substance inhibiting the function of WNT-5A.

In still another embodiment, the present invention provides a method of selecting a substance inhibiting the function of WNT-5A that is a method of screening a dermal papilla cell growth promoter, which comprises the following steps (a) to (c):
(a) a step of culturing human WNT-5A-expressing cells in a medium to which a test compound has been added;
(b) a step of lysing the human WNT-5A-expressing cells cultured in the step (a) to extract RNA and determining the WNT-5A mRNA amount; and
(c) a step of comparing the WNT-5A mRNA amount determined in the step (b).

### BRIEF DESCRIPTION OF THE DRAWINGS

FIG. 1 shows an electrophoresis pattern demonstrating that WNT-5A mRNA is expressed in dermal papilla cells.

FIG. 2 shows an electrophoresis pattern demonstrating that test compounds cause decreases in the WNT-5A mRNA amount in dermal papilla cells.

FIG. 3 shows a graph demonstrating that test compounds have an activity of promoting the growth of dermal papilla cells.

FIG. 4 shows an electrophoresis pattern demonstrating decreases in WNT-5A mRNA amount in monkey skin organ culture.

FIG. 5 shows the results of PCNA-staining in telogen and anagen hair follicles in monkey skin organ culture.

FIG. 6 shows histograms demonstrating that Compound 7 enlarges the hair bulb diameter in monkey skin organ culture.

FIG. 7 shows histograms demonstrating that Compound 3 enlarges the hair bulb diameter in monkey skin organ culture.

FIG. 8 shows photos of the skin before and after application of test compound for 6 months in a hair growth test on a stumptailed macaque (Macaca arctoides).

### BEST MODE FOR CARRYING OUT THE INVENTION

Now, the present invention is described in greater detail.

Compound Inhibiting the Function of WNT-5A

The term “a compound inhibiting the function of WNT-5A” (hereinafter sometimes referred to as “WNT-5A function inhibitor”) as used in the present invention means a compound inhibiting the binding of WNT-5A to a WNT-5A receptor or a compound inhibiting the production of WNT-5A, preferably a compound inhibiting the production of WNT-5A.

It has been confirmed that WNT-5A, which is a secretory glycoprotein belonging to the WNT family, is expressed in human, mouse, rat, Xenopus laevis and the like. From the viewpoint of using as a drug, a compound inhibiting the function of human WNT-5A (SEQ ID NO:1) is preferred.

The compound inhibiting the binding of WNT-5A to a WNT-5A receptor means a compound which acts on WNT-5A or the WNT-5A receptor and thus inhibits the binding of WNT-5A to the WNT-5A receptor to thereby regulate the signal transduction by WNT-5A, and is preferably a compound regulating the signal transduction via the Ca²⁺ pathway. Examples thereof include a WNT-5A receptor antagonist. Specific examples of the WNT-5A receptor include human Frizzled 5 (SEQ ID NO:4) and rat Frizzled 2 (SEQ ID NO:6). Although such a compound may be either peptidic or nonpeptidic, a nonpeptidic inhibitor is preferred because of its advantageous of having a longer duration of action. The compound can be selected by a screening system using a labeled WNT-5A and WNT-5A receptor, and has preferably an IC₅₀ of 30 μM or less, and more preferably IC₅₀ of 10 μM or less.

The compound inhibiting the production of WNT-5A means a compound inhibiting the expression of WNT-5A gene. Although such a compound may be either peptidic or nonpeptidic, a nonpeptidic inhibitor is preferred because of its advantageous of having a longer duration of action. The compound can be selected by using a decrease in the amount of WNT-5A protein (SEQ ID NO:1) or the amount of WNT-5A mRNA (SEQ ED NO:2) as an index, and has preferably an IC₅₀ of 30 μM or less, and more preferably 10 μM or less by a nucleic acid probe assay in accordance with the method of Hartley et al. (Drug Metabolism and Disposition, 28(5), 608-616 (2000); Test Example 4 described below).

Compounds represented by formulae (I), (II) and (IX) are particularly preferred.

In the compounds represented by formulae (I), (II) and (IX), the C₁₋₆ alkyl group means a linear or branched alkyl group having from 1 to 6 carbon atoms, and examples thereof include a methyl group, an ethyl group, a propyl group, an isopropyl group, a butyl group, an isobutyl group, a tert-butyl group, a pentyl group, a 2-ethylpropyl group, a hexyl group, and the like.

The C₁₋₆ alkanoyl group means a linear or branched alkanoyl group having from 1 to 6 carbon atoms, and examples thereof include an acetyl group, a propionyl group, a butyryl group, a t-butyryl group and the like.

The C₁₋₆ alkoxy group means a linear or branched alkoxy group having from 1 to 6 carbon atoms, and examples thereof include a methoxy group, an ethoxy group, a propoxy group, an isopropoxy group, a butoxy group, an isobutoxy group, a sec-butoxy group, a tert-butoxy group, a pentyloxy group, an isopentyloxy group, a neoisopentyloxy group, a tert-pentyloxy group, a 1-methylbutoxy group, a 2-methylbutoxy group, a 1,2-dimethylpropoxy group, a hexyloxy group, an isohexyloxy group and the like.

The C₁₋₆ alkoxyimino group means a linear or branched alkoxyimino group having from 1 to 6 carbon atoms, and examples thereof include an N-methoxyimino group, an N-ethoxyimino group, an N-propxyimino group, an N-isopropoxyimino group, an N-butoxyimino group, an N-isobutoxyimino group, an N-pentyloxyimino group, an N-hexyloxyimino group, and the like.

The C₁₋₆ alkanoyloxy group means a linear or branched alkanoyloxy group having from 1 to 6 carbon atoms, and examples thereof include an acetoxy group, a propionyloxy group, a pivaloyloxy group, and the like.

The C₃₋₁₀ cycloalkyl group means a cycloalkyl group having from 3 to 10 carbon atoms, and examples thereof include a cyclopropyl group, a cyclobutyl group, a cyclopentyl group, a cyclohexyl group, a cycloheptyl group, a cyclooctyl group, a cyclononyl group, and the like.

Examples of the substituted C₃₋₁₀ cycloalkyl group include a cycloalkyl group wherein a hydrogen atom(s) in the cycloalkyl group is/are substituted with at least one group selected from the group consisting of a halogen atom, an substituted or unsubstituted C₁₋₁₀ alkyl group, a hydroxyl group, a C₁₋₅ hydroxyalkyl group, a carboxyl group, a mercapto group, an indolyl group, a C₁₋₅ alkylthio group, an amino group, an amido group and a C₁₋₅ alkoxy group.

Examples of the aryl group include a phenyl group, a naphthyl group, an anthracene group and the like.

Examples of the substituted aryl group include aryl groups wherein a hydrogen atom(s) in an aryl group is/are substituted with at least one group selected from the group consisting of a substituted or unsubstituted C₁₋₁₀ alkyl group, a hydroxyl group, a C₁₋₅ hydroxyalkyl group, a carboxyl group, a mercapto group, an indazolyl group, an indolyl group, a C₁₋₅ alkylthio group, a cyano group, a nitro group, an amino group, an amido group, an acetylamino group and a C₁₋₅ alkoxy group.

Examples of the C₂₋₁₀ heterocycle include furan, pyrrole, thiophene, oxazole, isoxazole, thiazole, imidazole, pyrazole, pyran, pyridine, piperidine, pyridazine, pyrimidine, pyrazine, quinoline and the like.

Examples of the substituted C₂₋₁₀ heterocycle include a heterocycle wherein a hydrogen atom(s) in the heterocycle is/are substituted with at least one group selected from the group consisting of a halogen atom, a substituted or unsubstituted C₁₋₁₀ alkyl group, a hydroxyl group, a C₁₋₅ hydroxyalkyl group, a carboxyl group, a mercapto group, an indazolyl group, an indolyl group, a C₁₋₅ alkylthio group, a cyano group, a nitro group, an amino group, an amido group and a C₁₋₅ alkoxy group.

The ether bond means —O—, the formula —(CH₂)ₘ—O—(CH₂)ₙ— (wherein m and n each represents an integer of 1 to 3 and the alkylene groups in the formula may be substituted with an alkyl group(s)), or the formula —O—(CH₂)ₘ—-O— (wherein m is an integer of 1 to 3 and the alkylene groups in the formula may be substituted with an alkyl group(s)).

Examples of the combination of the adjacent groups which form a π bond or an ether bond include (R⁴ with R⁵), (R⁵ with R₆), (R⁶ with R⁷), (R⁷ with R⁸) and (R⁸ with R⁹) (the same applies to R^{4a} to R^{9a} in formula (IX)).

Among the compounds represented by formula (IX), a compound represented by formula (I) is preferred, and a compound wherein R^{3a} is a hydrogen atom and R^{3b} is a hydroxyl group is more preferred. From the viewpoint of the stability of compound, compounds represented by formulae (IIa), (IIb) and (IIc) are preferred.

Compound of formula (II) wherein (R⁴ and R⁵) and (R⁶ and R⁷) each form a π bond. 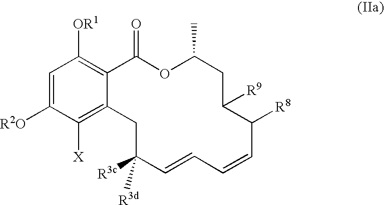
(wherein R¹, R², R^{3c}, R^{3d}, R⁸, R⁹ and X have the same meanings as described above).

Compound of formula (II) wherein (R⁴ and R⁵) form a π bond and R⁶ and R⁷ each represents a hydrogen atom: 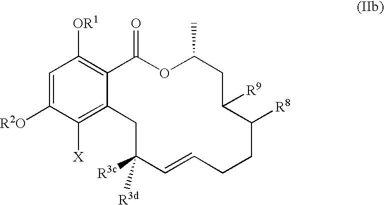
(wherein R¹, I², R^{3c}, R^{3d}, R⁸, R⁹ and X have the same meanings as described above).

Compound of formula (II) wherein R⁴, R⁵, R⁶ and R⁷ each represents a hydrogen atom: 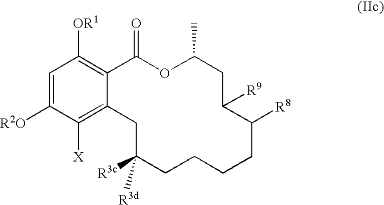
(wherein R¹, R², R^{3c}, R^{3d}, R⁸, R⁹ and X have the same meanings as described above).

Among the compounds represented by formula (IX), a compound wherein R^{3e} and R^{6b} are taken together to form an ether bond and (R^{3f} and R^{4a}) and (R^{5a} and R^{6a}) each form a ix bond (e.g., Compound 77, etc) are preferred since they effectively inhibit the expression of WNT-5A mRNA in dermal papilla cells and promote the growth of the dermal papilla cells.

Among the compounds represented by formula (IX), a compound wherein R^{1a} and R^{2a} each represents (CH₂)ₚCO—Y—R¹⁰ (wherein Y represents an oxygen atom or a sulfur atom; R¹⁰ represents a hydrogen atom, a C₁₋₆ alkyl group or a substituted or unsubstituted aryl group; and p represents 0 or 1) (e.g., Compound 52, etc.) and a compound wherein R^{1a} and R^{2a} represent each COR₁₂ (wherein R₁₂ represents a substituted or unsubstituted aryl group or a substituted or unsubstituted heterocycle) (e.g., Compound 58, etc.) are preferred since they effectively inhibit the expression of WNT-5A mRNA in dermal papilla cells and promote the growth of the dermal papilla cells.

Also, a compound wherein R^{7a} represents Z-R¹³ (wherein Z represents an oxygen atom or a sulfur atom; and R¹³ represents a C₂₋₆ alkanoyl group or a substituted or unsubstituted aryl group) (e.g. Compound 53, etc.) is preferred since they effectively inhibit the expression of WNT-5A mRNA in dermal papilla cells and promote the growth of the dermal papilla cells.

A compound represented by formula (II) can be produced by, for example, combining the following production methods. 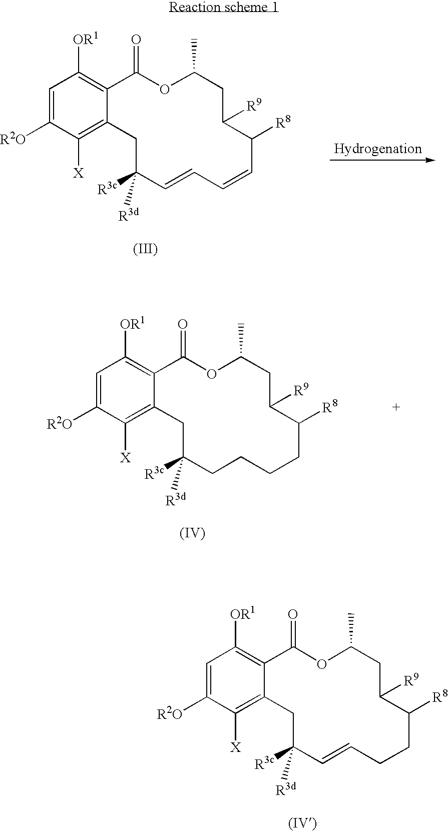
(wherein R¹, R², R^{3c}, R^{3d}, R⁸, R⁹ and X have the same meanings as described above).

The compound represented by formula (III) is subjected to a hydrogenation reaction in the presence of a catalyst such as palladium carbon in an organic solvent (e.g., ethyl acetate, tetrahydrofuran, diethyl ether, ethyl alcohol, methyl alcohol, etc.) to give the compound represented by formula (IV) or (IV′) or a mixture of these compounds. The compounds of formulae (IV) and (IV′) can be separated and purified by a separation method commonly employed such as column chromatography. 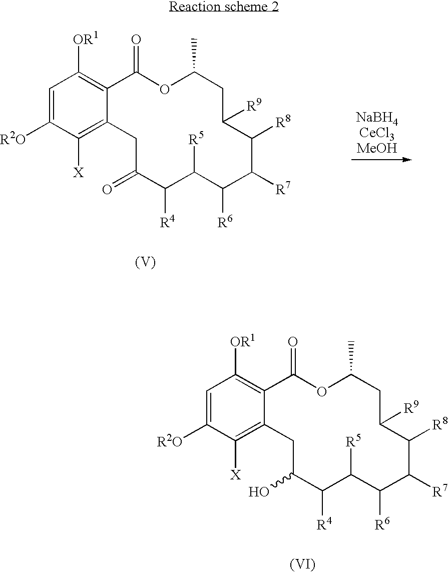
(wherein R¹, R², R⁴, R⁵, R⁶, R⁷, R⁸, R⁹ and X have the same meanings as described above).

The compound represented by formula (V) is allowed to react with a reducing agent such as potassium boron hydride, sodium boron hydride or lithium boron hydride, if necessary, in the coexistence of a salt such as LiCl, MgCl₂, CeCl₃, CaCl₂ or NiCl in an organic solvent (e.g., tetrahydrofuran, diethyl ether, ethyl alcohol, methyl alcohol, etc.) at −20 to 100° C., preferably at 0 to 20° C., to give the compound represented by formula (VI). The compound represented by formula (VI) can be separated and purified by a separation method commonly employed such as column chromatography. 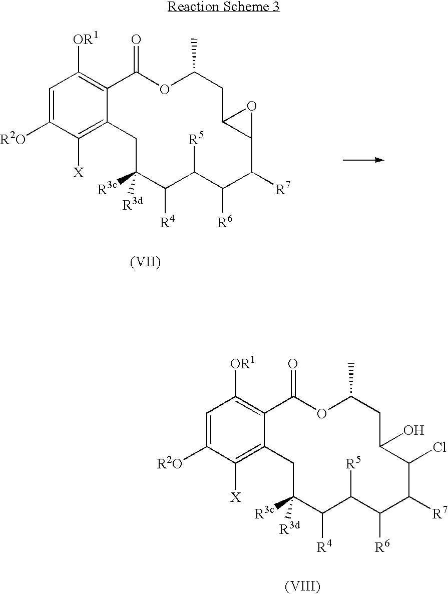
(wherein R¹, R², R^{3c}, R^{3d}, R⁴, R⁵, R⁶, R⁷ and X have the same meanings as described above).

The compound represented by formula (VII) is treated with an acid or a base in an appropriate organic solvent (e.g., tetrahydrofuran, diethyl ether, ethanol, methanol, etc.) to give the compound represented by formula (VIII).

The compounds represented by formula (IX) (1, 60 to 78) are produced by culturing Pochonia chalamydosporia var. chlamydosporia TF-0480 strain in a medium containing various nutrients basically in accordance with methods commonly employed for producing fermentation products.

A liquid medium is mainly used as the medium, the medium contains a carbon source, a nitrogen source and an inorganic salt optionally together with vitamins, precursors and a defoaming agent, and the pH is adjusted to around 7. The carbon source includes glucose, dextrin, glycerine, starch and the like, which may be used alone or as a mixture thereof. The nitrogen source includes meat extract, oatmeal, yeast extract, soybean flour, polypeptone, corn steep liquor, urea, an ammonium salt and the like, which may be used alone or as a mixture thereof. The inorganic salt includes monopotassium phosphate, magnesium sulfate, sodium chloride, calcium carbonate and the like, which may be used alone or as a mixture thereof. The defoaming agent which can be used include Adekanol or a silicone compound. As the culture method, aerobic culture methods such as shaken culture and aeration-agitation culture are suitable. The culture is carried out at pH 4 to 10 and at 25 to 35° C. for 2 to 5 days, preferably at 25 to 28° C. for 3 days.

Compounds 1 and 60 to 78 thus produced by the culture can be isolated in accordance with methods commonly employed for collecting fermentation products. For example, it is effective to employ the following methods. Namely, after the completion of the culture, a culture filtrate is obtained by centrifugation or filtration. Then, it is adsorbed by a polystyrene resin such as DIAION RP-20 (trade name, manufactured by Mitsubishi Chemical) and eluted with an organic solvent such as lower alcohol or acetone. Cells are extracted with an organic solvent such as lower alcohol or acetone. Next, the cell extract and the eluate from the adsorbent resin are combined together and concentrated under reduced pressure. To the remaining water phase, an organic solvent such as ethyl acetate, chloroform or n-butanol is added for extraction, and the extract is concentrated. The resulting residue is redissolved in an organic solvent such as benzene, ethyl acetate, acetone, methanol or chloroform and then applied to silica gel column chromatography, gel filtration column chromatography, column chromatography packed with ODS for reversed phase partition and high performance liquid chromatography to thereby purify and isolate the compound according to the present invention.

The strain producing the active ingredient according to the present invention is a novel strain that has been newly isolated from soil by the present inventors. The microorganism was named “Pochonia chlamydosporia var. chlamydosporia TF-0480” and has been deposited to International Patent Organism Depository, National Institute of Advanced Industrial Science and Technology as “FERM BP-8332”.

The microbiological properties of this strain are as follows.

1) Growth on Medium

The microorganism grows well in various agar plate media and well or moderately forms spores in tested media. Table 1 summarizes characteristics of colonies formed after culturing at 26° C. for 2 weeks on various media. Colors are indicated in accordance with the color codes proposed by A. Kornerup ex al., Dizionario dei colori, Musterschmidt (1978).

TABLE 1

| Medium | Results of observation on colonies with naked eye |
|---|---|
| PDA*⁻¹ | Showing relatively well growth and sporulation. Colony diameter: 23-28 mm. |
| | Wooly and somewhat domed colonies with pannose center and slight |
| | radial wrinkles. Somewhat irregular periphery (colony tip). Secreting a |
| | pale light yellow [3A6] pigment into the medium. Colony surface color: |
| | white [1A1] at the periphery and pale yellow [4A3] at the center. Back |
| | face color: reddish yellow [4A6] at the periphery and yellowish brown |
| | [5D8] at the center. Agar medium being irregularly or radially fractured. |
| OMA*⁻² | Showing well growth and sporulatian. Colony diameter: 48-50 mm. |
| | Somewhat thin and wooly colonies with domed center and intermediate |
| | part. Relatively regular-shaped periphery. No pigment secretion. |
| | Colony surface color: white [1A1] at the periphery and light yellow [3A4] at |
| | the center. Back face color: light yellow [3A4] similar to the surface. |
| CMA*⁻³ | Showing well growth and moderate sporulation. Colony diameter: 47-51 mm. |
| | Thin and pannose colonies. Relatively regular-shaped periphery. |
| | No pigment secretion into the medium. Colony surface/back face color: |
| | both white [1A1] to yellowish white [1A2]. |
| MA*⁻⁴ | Showing relatively well growth and sporulation. Colony diameter: 26-27 mm. |
| | Thick and wooly colonies with domed center. Relatively regular- |
| | shaped periphery. Secreting a slightly reddish yellow [4A7] pigment into |
| | the medium. Colony surface color: white [1A1] at the periphery and pale |
| | yellow [4A3] at the center. Back face color: light orange [5A4] at the |
| | periphery, brown [7E8] at the center and orange [6B7] at the intermediate |
| | part. |
| SA*⁻⁵ | Showing relatively well growth and sporulation. Colony diameter: 23-28 mm. |
| | Thick and wooly colonies with domed center. Somewhat irregular |
| | periphery with slight radial wrinkles. Secreting a reddish yellow [4A7] |
| | pigment into the medium. Colony surface color: white [1A1] at the |
| | periphery and orange white [5A2] in other parts. Back face color: light |
| | orange [5A4] at the periphery and center and brown [7E8] at the |
| | intermediate part. Agar medium being irregularly or radially fractured. |
| LCA*⁻⁶ | Showing well growth and moderate sporulation. Colony diameter: 42-44 mm. |
| | Thin pannose to slightly wooly colonies. Relatively regular-shaped |
| | periphery. No secretion of pigment into the medium. Colony |
| | surface/back face color: both white [1A1]. |

*⁻¹Potato dextrose agar medium (E-MF21, manufactured by Eiken Chemical Co., Ltd.).
*⁻²½-Diluted oatmeal agar medium (manufactured by Difco, prepared by ½-diluting Bacto oatmeal agar and adding agar to give a final agar concentration of 2%).
*⁻³Corn meal agar medium (manufactured by Difco, Corn meal agar).
*⁻⁴Malt extract agar medium (Nakase, T., 6th ed., pp.617, Japan Collection of Microorganisms, the Institute of Physical and Chemical Research, Saitama, 1995)
*⁻⁵Sabouraud agar medium (manufactured by Eiken Chemical Co., Ltd., E-MF03)
*⁻⁶Miura agar medium (Miura, K. and M. Kudo, Trans. Mycol. Soc. Japan, 11, 116-118 (1970))

2) Morphology

Colonies formed after culturing the strain on a corn meal agar plate at 26° C. for 14 days are observed under an optical microscope. As a result, the formation of phialo-conidia was observed. A single conidium-forming cell (phialide) or 2 to 4 verticillate phialides are formed directly on a hypha. Conidiophorer cannot be clearly distinguished from vegetative hyphae. A plural number of conidia are formed at the front edge of a phialide to form a viscous mass, A phialide has a tapered cylindrical shape and smooth and colorless surface. It is from 13 to 15 μm in length and 0.8 to 1.7 μm in thickness at the base and 0.5 to 0.8 μm in thickness at the end. Conidia are in an elliptical shape or a semi-spherical shape (rare) and most of them have slightly projecting base. The conidia have smooth and colorless surface and are 2.2 to 5.0×1.8 to 2.8 μm in size. A single pale yellow and multicellular chlamydospore having netted wall (dictyochlamydospore) is formed on the medium surface or at the end of a short hypha sprouting on the medium. Dictyochlamydospores are also formed in the medium in a small amount. These dictyochlamydospores are 14 to 20×12 to 22 μm in size. After prolonging the culture over one month or longer, no teleomorph is observed.

3) Physiological Properties

(1) Growth Temperature Range and Optimum Temperature

In Sabouraud liquid medium at pH 6.0, this strain grows within a temperature range of 15 to 31° C. and the optimum temperature is from 25 to 27° C.

(2) Growth pH Range and Optimum pH Value

In YpSs liquid medium at 26° C., this strain grows within a pH of 3 to 10 and the optimum pH is from 5 to 7.

4) Aerobic/anaerobic: aerobic.

Based on the above-described morphological characteristics and culture properties, the characteristics of this strain are compared with various known species reported in (1) K. H. Domsch et al., Compendium of soil fungi, Vol. 1, IHW-Verlag (1980) and (2) G. L. Barron, The Genera of Hyphoomycetes from soil, Williams & Wilkins (1968) and the like. As a result, this strain is closely similar to an incomplete species known under a classification name Verticillium chlamydosporia or Diheterospora chlamydosporia and, therefore, seemingly belongs to the same species. In (3) R. Zare, W. Gain and H. C. Evans, A revision of Verticillium section Prostrata. V. The genus Pochonia, with notes on Rotiferophthora, 73, 1-2, p 51-86 (2001), this species is molecular systematically re-examined and, in its turn, referred to as belonging to the genus Pochonia as the proper genus. Moreover, it is suggested to refer such a species forming non-linked Verticillium-type conidia as the strain of the present invention as to a variant Pochonia chlamydosporia var. chlamydosporia.

Based on these facts, this strain is named “Pochonia chlamydosporia var. chlamydosporia TF-0480”.

Dermal Papilla Cell Growth Promoter

The term “dermal papilla cell growth promoter” as used in the present invention means a medicament or a reagent having an effect of increasing the number of dermal papilla cells.

The dermal papilla cell growth promoter according to the present invention is characterized by being based on the effect of inhibiting the function of WNT-5A. Since WNT-5A inhibits the functions of WNT-1 class molecules (WNT-1, WNT-8, etc.) which are activators of WNT/β-catenin pathway, the growth of dermal papilla cells is controlled by regulating the function of WNT-5A or regulating the expression of WNT-5A. Therefore, even compounds having completely different structures (e.g., Compound 24, Compound 79, Compound 7, etc.) have an excellent effect of promoting the growth of dermal papilla cells, so long as these compounds have an excellent effect of inhibiting the function of WNT-5A.

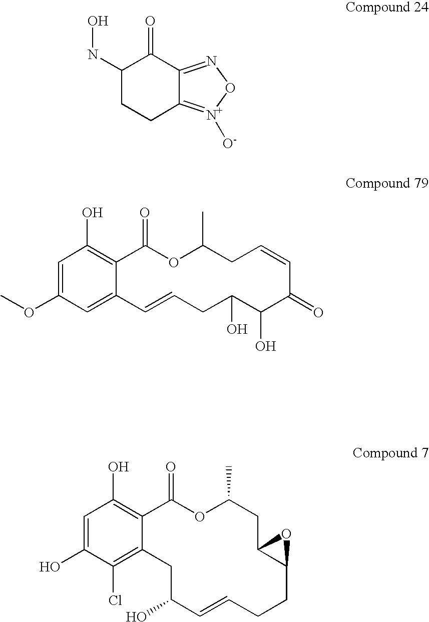

The growth of cells can be measured by a method publicly known by a person skilled in the art. Examples of the measuring method include counting of viable cells using an appropriate color-developing substrate and the [³H]-thymidine uptake method. As the color-developing substrate, tetrazolium salts such as MTT, MTS and XTT and Alamar Blue are preferably used.

Hair Growth Stimulant/Hair Growth Tonic

The term “hair growth stimulant or hair growth tonic” as used in the present invention means products to be used for inducing hair growth, promoting hair growth, preventing alopecia and the like. In the case of using the hair growth stimulant and hair growth tonic according to the present invention as medicaments, the subject to be applied includes improvement or prevention of alopecia areata and male pattern alopecia.

The effects of the hair growth stimulant and hair growth tonic according to the present invention are achieved by the effect of promoting the growth of dermal papilla cells by inhibiting the function of WNT-5A. Based on this function mechanism, the cell growing ability, which has been depressed in dermal papillae in an alopecia site, is enhanced and dermal well-developed papilla tissue is formed. It is therefore expected that the hair growth stimulant/hair growth tonic according to the present invention are efficacious against symptoms on which little effects can be established by the existing hair growth stimulants and hair growth tonics.

It is furthermore expected that synergistic effects could be obtained by combining the hair growth stimulant and hair growth tonic according to the present invention with other hair growth stimulants and hair growth tonics having different function points.

The hair growth stimulant and hair growth tonic according to the present invention can be administered in various doses and various dosage forms depending on individual compounds.

In the case of administering the compound represented by formula (IX) by application (lotion, ointment, a gel, etc.), for example, it can be administered at a dose of 0.0001 to 10% by weight, preferably 0.001 to 5% by weight and more preferably 0.001 to 1% by weight, although the dose varies depending on the type and dosage form of the hair growth tonic. In the case of orally administering (dust, tablet or capsule) the compound represented by formula (IX) to a male adult, the daily dose preferably ranges from 1 to 100 mg/kg.

Although the dosage forms of the hair growth stimulant and hair growth tonic according to the present invention are not particularly restricted, it is preferable to provide the hair growth stimulant/hair growth tonic containing a WNT-5A production inhibitor (e.g., the compound represented by formula (IX)) as an active ingredient in the form of a water-soluble composition in the case of external application. Such a water-soluble composition can be produced by using various additives (humectant, thickener, preservative, antioxidant, aromatic coloring agent, etc.) commonly employed in producing drugs, quasi drugs or cosmetics, so long as the effects of the present invention are not damaged thereby. The hair growth stimulant and hair growth tonic according to the present invention can be provided as hair-care compositions such as hair tonic, hair oil, hair moose or gel, or ointments.

In the case of using hair growth stimulant and hair growth tonic according to the present invention as liquid preparations, such a preparation is produced as an aqueous solution, a non-aqueous solution, a suspension, liposomes or an emulsion prepared by dissolving a WNT-5A production inhibitor (e.g., the compound represented by formula (IX)) in purified water, an appropriate buffer such as a phosphate buffer, a physiological salt solution such as physiological saline, Ringer solution or Locke solution, ethanol or glycerol appropriately combined with a surfactant, etc. commonly employed, followed by sterilization. It is topically administered as a liquid preparation for scalp. The liquid preparation may be applied directly to the scalp. Alternatively, it may be applied by using a jetting nozzle such as a spray.

In the case of using hair growth stimulant and hair growth tonic according to the present invention as semi-solid preparations, a WNT-5A production inhibitor (for example, the compound represented by formula (IX)) is mixed with a fat, an oil, lanolin, vaseline, paraffin, wax, a plaster, a resin, plastic, glycerine, higher alcohol, glycerol, water, an emulsifier, a suspending agent or the like to give an external preparation (ointment, cream, etc.) which can be topically administered.

In the case of using hair growth stimulant and hair growth tonic according to the present invention as solid preparations, a WNT-5A production inhibitor (e.g., the compound represented by formula (IX)) is mixed with appropriate additive(s) to give an external preparation such as a dust or a powder. Alternatively, it is possible to produce a solid preparation which is dissolved or suspended in a solvent before using and applied to the scalp.

In the case of orally using, it is preferable that a WNT-5A production inhibitor (e.g., the compound represented by formula (IX)) is blended with a pharmaceutically acceptable carrier (filler, binder, disintegrating agent, flavor, corrigent, emulsifier, etc.), a diluent, a dissolution aid or the like and the obtained medicinal composition is processed in a conventional manner to give a tablet, a capsule, granules, a dust, a syrup, a suspension, a solution and the like.

These preparations can be produced by using formulation techniques commonly employed.

Screening Method

The present invention further relates to a method of screening a dermal papilla cell growth promoter, which comprises selecting a compound inhibiting the function of WNT-5A.

Selecting a compound inhibiting the function of WNT-5A (hereinafter sometimes referred to as “WNT-5A function inhibitor”) means, for example, selecting (i.e., screening) a WNT-5A production inhibitor, or selecting a WNT-5A receptor antagonist.

As test materials to be subjected to the screening method according to the present invention, any materials can be used. That is, the test materials are not restricted in kind, and they may be low-molecular weight compounds, compounds contained in a natural extract or synthetic peptides. They may also be a compound library or a combinatorial library. The compound library can be constructed by a method publicly known by one of the skilled in the art. Alternatively, a commercially available compound library can be used. From the viewpoint of using as a medicament, the compound which is subjected to screening preferably has a molecular weight of 3000 or less. From the viewpoint of enabling application/oral administration, a low-molecular weight compound having a molecular weight of 600 or less is preferred.

(1) Method of Screening WNT-5A Receptor Antagonist

In a method of screening a WNT-5A receptor antagonist, whether or not a bond between the WNT-5A protein and the WNT-5A receptor is formed can be examined by using a labeled WNT-5A protein and a WNT-5A receptor and detecting or measuring the label. Examples of the label include a radioisotope (³²P, ³³P, ¹³¹I, ¹²⁵I, ³H, ¹⁴C, ³⁵S, etc.), an enzyme (alkali phosphatase, hoarse radish peroxidase, etc.), a fluorescent substance (fluorescein isothiocyanate, etc.) and the like. They can be commercially available and the labeling can be made by a known method.

A specific example of an in vitro assay system is performed in a cell-free system. More specifically, one of the WNT-5A protein and the WNT-5A receptor is bound to a support and the other and a test material are added thereto. After incubating, the reaction mixture is washed and the binding of the other protein to the protein having been bound to the support is detected or measured.

Examples of the support to which the protein is bound include insoluble polysaccharides such as agarose, dextran and cellulose; synthetic resins such as polystyrene, polyacrylamide and silicone; and the like. More specifically, commercially available beads and plates obtained by these materials can be employed.

(2) Method of Screening Compound Inhibiting WNT-5A Production

A compound inhibiting WNT-5A production can be screened by using the amount of WNT-5A mRNA or the amount of WNT-5A protein as an index. It is also possible to bind a reporter gene to the promoter region of a WNT-5A gene and detect the expression amount. As the promoter of the WNT-5A gene, one represented by SEQ ID NO:3 is preferably used.

Examples of the reporter gene include a GFP green fluorescent protein) gene, a GUS (β-glucuronidase) gene, an LUC (luciferase) gene, and a CAT (chloramphenicol acetyltransferase) gene.

When the expression amount of WNT-5A mRNA is used as an index, for example, human WNT-5-expressing cells are used and a test material is added thereto. After culturing in an incubator for several hours at 37° C. in a 5% CO₂-95% air atmosphere, the cells are lysed and RNAs are extracted. Then, the amount of the WNT-5A RNA is measured by, for example, RT-PCR. Thus, a material having an activity of decreasing the WNT-5mRNA amount can be searched. The primers used in PCR are not particularly limited, so long as they are specific to WNT-5A mRNA, and they can be designed from the WNT-5A mRNA sequence. Preferred examples include a forward primer AATGTCTTCC AAGTTCTTCC TAGTGGC (SEQ ID NO:8) and a reverse primer GATGTCGGAA TTGATACTGG CA (SQ ID NO:9).

When the WNT-5A protein amount is used as an index, for example, human WNT-5A-expressing cells are used and a test material is added thereto. After culturing in an incubator for several hours at 37° C. in a 5% CO₂-95% air atmosphere, the cells are lysed by using the culture medium and proteins are extracted. Then, the amount of the WNT-5A protein is measured by ELISA (enzyme-linked immunosorbent assay) or the like. Thus, a substance having an activity of decreasing the expression amount of the WNT-5A mRNA can be searched.

Next, the present invention is described in detail by reference to Test Examples below, however, the present invention is not limited thereto.

### EXAMPLE 1

### Compounds 3 and 4

Radicicol (Compound 1: 10.8 g) was dissolved in ethyl acetate (140 ml) and 5% palladium carbon (wet type) (255 mg) was added thereto, followed by hydrogen replacement (1 atm) and stirring at room temperature for 3 hours. After filtering the palladium carbon, the filtrate was distilled off under reduced pressure. The resulting residue was purified by silica gel chromatography and eluted with n-hexane:ethyl acetate (2:1) to give target Compound 4 (3.43 g) was obtained, or eluted with n-hexane:ethyl acetate (3:2) to give target Compound 3 (4.14 g).

### EXAMPLE 2

### Compounds 7 and 8

Compound 3 (602.5 mg) was dissolved in methanol (13 ml) and cerium (III) chloride heptahydride (2.14 g) was added thereto, followed by stirring at room temperature for 30 minutes. Next, sodium boron hydride (180 mg) was slowly added to the solution under ice cooling, followed by stirring at room temperature for 5 minutes. After adding saturated disodium hydrogen phosphate (40 ml) to the reaction solution, the mixture was diluted with water (40 ml) and the organic solvent was distilled off under reduced pressure. The residual aqueous layer was extracted with ethyl acetate (300 ml×2). The resulting ethyl acetate layer was dried over anhydrous sodium sulfate and then the ethyl acetate was distilled off under reduced pressure. Then, the residue was crudely purified by silica gel preparative thin layer chromatography (20 cm×20 cm, 1.0 mm in thickness, developed with chloroform:methanol=93:7, eluted with ethyl acetate). The resulting crude purified product was then purified by high performance liquid chromatography (20φ×250 mm, YMC-Pack Pro C18, eluted with water (to which acetic acid had been added, pH 3.5):acetonitrile=65:35) to give the target compounds (Compound 7: 144.9 mg, Compound 8: 13.8 mg).

### EXAMPLE 3

### Compounds 9 and 10

Compound 4 (26.5 mg) was dissolved in methanol (5 ml) and cerium (M) chloride heptahydride (100 mg) was added thereto, followed by stirring at room temperature for 30 minutes. Ne sodium boron hydride (60 mg) was slowly added to the solution under ice cooling, followed by stirring at room temperature for 30 minutes. After adding saturated disodium hydrogen phosphate (12 ml) to the reaction solution, the mixture was diluted with water (20 ml) and the organic solvent was distilled off under reduced pressure. The residual aqueous layer was extracted with ethyl acetate (30 ml×2). The resulting ethyl acetate layer was dried over anhydrous sodium sulfate and then the ethyl acetate was distilled off under reduced pressure. Then, the residue was purified by silica gel preparative thin layer chromatography (20 cm×20 cm, 0.25 mm in thickness, developed with chloroform:methanol=94:6, eluted with ethyl acetate) to give the target compounds (Compound 9: 5.7 mg, Compound 10: 8.8 mg).

### EXAMPLE 4

### Compound 5

Radicicol (91.5 mg) was dissolved in methanol (5 ml) and cerium (III) chloride heptahydride (88 mg) was added thereto, followed by stirring at room temperature for 10 minutes. Next, sodium boron hydride (60 mg) was slowly added to the solution under ice cooling, followed by stirring at room temperature for 30 minutes. After adding saturated disodium hydrogen phosphate (20 ml) to the reaction solution, the mixture was diluted with water (20 ml) and the organic solvent was distilled off under reduced pressure. The residual aqueous layer was extracted with ethyl acetate (50 ml×3). The resulting ethyl acetate layer was dried over anhydrous sodium sulfate and then the ethyl acetate was distilled off under reduced pressure. Then, the residue was purified by silica gel preparative thin layer chromatography (20 cm×20 cm, 0.5 mm in thickness, developed with chloroform:methanol=9:1, eluted with ethyl acetate) to give target Compound 5 (27.2 mg).

### EXAMPLE 5

### Compound 2

Radicicol (15.3 mg) was dissolved in pyridine (1.5 ml) and acetic anhydride (4 ml) was added thereto, followed by stirring at room temperature for 6.5 hours. After adding ice water (20 ml) to the reaction solution, the mixture was extracted with ethyl acetate (20 ml). The ethyl acetate layer was washed with water (20 ml×2) and dried over anhydrous sodium sulfate and then the ethyl acetate was distilled off under reduced pressure. Then, the residue was purified by silica gel preparative thin layer chromatography (20 cm×20 cm, 0.5 mm in thickness, developed with chloroform:methanol=95:5, eluted with ethyl acetate) to give target Compound 2 (18.5 mg).

### EXAMPLE 6

### Compound 11

Radicicol (19.0 mg) was dissolved in dimethyl sulfoxide (1 ml), and potassium carbonate (3 mg) and methyl iodide (4 ml) were added thereto, followed by stirring at room temperature for 6.5 hours. After adding water (20 ml) to the reaction solution, the mixture was extracted with ethyl acetate (20 ml). The ethyl acetate layer was dried over anhydrous sodium sulfate and then the ethyl acetate was distilled off under reduced pressure. Then, the residue was purified by silica gel preparative thin layer chromatography (20 cm×20 cm, 0.5 mm in thickness, developed with chloroform:methanol 95:5, eluted with ethyl acetate) to give target Compound 11 (15.3 mg).

### EXAMPLE 7

### Compounds 16, 17, 19 and 23

Radicicol (930 mg) was dissolved in 1,4-dioxane (14 ml) and 1N hydrochloric acid (12 ml) was added thereto, followed by stirring at room temperature for 2 hours. After diluting with water (40 ml), the mixture was extracted with ethyl acetate (100 ml). The ethyl acetate layer was dried over anhydrous sodium sulfate and then the ethyl acetate was distilled off under reduced pressure. Then, the residue was crudely purified by silica gel preparative thin layer chromatography (20 cm×20 cm, 1.0 mm in thickness, developed with chloroform:methanol:n-hexane=5:1:5, eluted with ethyl acetate). The resulting crude purified product was then purified by high performance liquid chromatography (20φ×250 mm, YMC-Pack Pro C18, eluted under gradient with water (to which acetic acid had been added, pH 3.5):acetonitrile=70:30 to 40:60) to give the target compounds (Compound 16: 11.4 mg, Compound 17: 19.4 mg, Compound 19: 32.6 mg, Compound 23: 103.7 mg).

### EXAMPLE 8

### Compounds 12 and 13

Radicicol (232.6 mg) was dissolved in 1,4-dioxane (4 ml) and 1N hydrochloric acid (1 ml) was added thereto, followed by stirring at room temperature for 30 minutes. After neutralizing with 1N sodium hydroxide, the solvent was distilled off under reduced pressure. Then, the concentrate was dissolved with 20 ml of methanol. The resultant solution was filtered through a cotton plug and the methanol was distilled off under reduced pressure. The resulting residue was then purified by high performance liquid chromatography (20φ×250 mm, YMC-Pack Pro C18, eluted with water (to which acetic acid had been added, pH 3.5):acetonitrile=65:35) to give the target compounds (Compound 12: 42,6 mg, Compound 13: 10.4 mg).

### EXAMPLE 9

### Compound 18

Into 5 ml of dimethylformamide, 1 ml of phosphorus oxychloride was dropped under ice cooling and then the mixture was stirred at room temperature for 30 minutes. The resultant solution was slowly added to a dimethylformamide solution (4 ml) of Radicicol (98.5 mg) under ice cooling followed by stirring at room temperature for 24 hours. Then, the reaction solution was diluted with ethyl acetate (100 ml) and washed with water (100 ml×3). The ethyl acetate layer was dried over anhydrous sodium sulfate and then the ethyl acetate was distilled off under reduced pressure. Then, the resulting residue was purified by silica gel preparative thin layer chromatography (20 cm×20 cm, 0.5 mm in thickness, developed with chloroform:methanol=94:6, eluted with ethyl acetate) to give the target compound (Compound 18: 61.8 mg).

### EXAMPLE 10

### Compounds 14 and 15

Radicicol (378 mg) was dissolved in 1,4-dioxane (4 ml) and 1N hydrochloric acid (1 ml) was added thereto, followed by stirring at room temperature for 20 minutes. After neutralizing with 1N sodium hydroxide, the solvent was distilled off under reduced pressure. Then, the concentrate was dissolved with 20 ml of methanol. The resultant solution was filtered through a cotton plug and the methanol was distilled off under reduced pressure. The resulting residue was then purified by high performance liquid chromatography (20φ×250 mm, YMC-Pack Pro C18, eluted with water (to which acetic acid had been added, pH 3.5):acetonitrile=70:30) to give the target compounds (Compound 14: 10.7 mg, Compound 15: 9.9 mg) simultaneously with Compound 12 (40.6 mg).

### EXAMPLE 11

### Compounds 20, 21 and 22

Compound 3 (96.3 mg) was dissolved in 1,4-dioxane (2 ml) and 1N hydrochloric acid (2.5 ml) was added thereto, followed by stirring at room temperature for 16 hours. After neutralizing with 1N sodium hydroxide, the solvent was distilled off under reduced pressure. Then, the concentrate was dissolved with 20 ml of methanol. The resultant solution was filtered through a cotton plug, and the methanol was distilled off under reduced pressure. The resulting residue was then purified by high performance liquid chromatography (20φ×250 mm, YMC-Pack Pro CIS, eluted with water (to which acetic acid had been added, pH 3.5):acetonitrile=45:55) to give the target compounds (Compound 20: 9.3 mg, Compound 21: 22.0 mg, Compound 22: 26.2 mg).

### EXAMPLE 12

### Compounds 25 and 26

Compound 7 (34 mg) was dissolved in N,N-dimethylformamide (2 ml), and potassium hydroxide (3 mg) and methyl iodide (500 μl) were added thereto, followed by stirring at room temperature for 4.5 hours. After adding water (20 ml) to the reaction solution, the mixture was extracted with ethyl acetate (20 ml). The ethyl acetate layer was dried over anhydrous sodium sulfate and the ethyl acetate was distilled off under reduced pressure. The resulting residue was dissolved in a small amount of acetone and purified by preparative high performance liquid chromatography [column: YMC-Pack Pro C18 AS-343 (20φ×250 mm), detection: UV absorption at 254 nm, flow rate; 10 ml/min] using acetonitrile-water (pH 3.5, acetic acid) (38:62) as the mobile phase to obtain two fractions. Each fraction was concentrated under reduced pressure to give Compound 25 (6.4 mg, 39 min) and Compound 26 (14.5 mg, 29 min).

### EXAMPLE 13

### Compound 27

Compound 7 (55 mg) was dissolved in pyridine (1 ml), and acetic anhydride (3 ml) was added thereto, followed by stirring at room temperature for 17 hours. After adding water (50 ml) to the reaction solution, the mixture was extracted with ethyl acetate (50 ml). The ethyl acetate layer was dried over anhydrous sodium sulfate and the ethyl acetate was distilled off under reduced pressure. The resulting residue was dissolved in a small amount of acetone and purified by preparative high performance liquid chromatography [column: YMC-Pack Pro CIS AS-343 (20φ×250 mm), detection: UV absorption at 254 nm, flow rate: 10 ml/min] using acetonitrile-water (pH 3.5, acetic acid) (60:40) as the mobile phase. The resulting fraction was concentrated under reduced pressure to give Compound 27 (64.7 mg, 18 min).

### EXAMPLE 14

### Compounds 28 and 29

Compound 8 (131 mg) was dissolved in N,N-dimethylformamide (2 ml), and potassium hydroxide (3 mg) and methyl iodide (2 ml) were added thereto, followed by stirring at room temperature for 7 hours. After adding water (40 ml) to the reaction solution, the mixture was extracted with ethyl acetate (40 ml). The ethyl acetate layer was dried over anhydrous sodium sulfate and the ethyl acetate was distilled off under reduced pressure. The resulting residue was dissolved in a small amount of acetone and purified by preparative high performance liquid chromatography [column: YMC-Pack Pro C18 AS-343 (20φ×250 mm), detection: UV absorption at 254 nm, flow rate; 10 ml/min] using acetonitrile-water (pH 3.5, acetic acid) (38:62) as the mobile phase. Thus 2 fractions were obtained and respectively concentrated under reduced pressure to give Compound 28 (14.2 mg, 52 min) and Compound 29 (31.7 mg, 37 min).

### EXAMPLE 15

### Compound 30

Compound 8 (72 mg) was dissolved in pyridine (2 ml), and acetic anhydride (2 ml) was added thereto, followed by stirring at room temperature for 17 hours. After adding water (50 ml) to the reaction solution, the mixture was extracted with ethyl acetate (50 ml). The ethyl acetate layer was dried over anhydrous sodium sulfate and the ethyl acetate was distilled off under reduced pressure. The resulting residue was dissolved in a small amount of acetone and purified by preparative high performance liquid chromatography [column: YMC-Pack Pro C18 AS-343 (20φ×250 mm), detection: UV absorption at 254 nm, flow rate; 10 ml/min] using acetonitrile-water (pH 3.5, acetic acid) (53:47) as the mobile phase. The obtained fraction was concentrated under reduced pressure to give Compound 30 (45.5 mg, 30 min).

### EXAMPLE 16

### Compound 31

Radicicol (10.8 g) was dissolved in ethyl acetate (140 ml) and 5% palladium carbon (wet type) (255 mg) was added thereto, followed by hydrogen replacement (1 atm) and stirring at room temperature for 3 hours. After filtering the palladium carbon, the filtrate was concentrated under reduced pressure. The resulting residue was purified by silica gel chromatography [Silica Gel 60 (trade name, manufactured by Merck)] and eluted with n-hexane:ethyl acetate (2:1) to give Compound 4 (3.43 g), or eluted with n-hexane:ethyl acetate (3:2) to give Compound 3 (4.14 g). A portion (500 mg) of a fraction (3.08 g) between Compound 3 and Compound 4 was dissolved in a small amount of N,N-dimethylformamide and purified by preparative high performance liquid chromatography [column: YMC-Pack Pro C18 AS-343 (20φ×250 mm), detection: UV absorption at 254 nm, flow rate: 10 ml/min] using acetonitrile-water (pH 3.5, acetic acid) (38:62) as the mobile phase. Thus 2 fractions were obtained and respectively concentrated under reduced pressure to give Compound 3 (177.8 mg) together with Compound 31 (20.7 mg, 23 min).

### EXAMPLE 17

### Compounds 32, 33 and 34

Radicicol (11.9 g) was dissolved in ethyl acetate (160 ml) and 5% palladium carbon (wet type) (300 mg) was added thereto. After purging with hydrogen (1 atm), the mixture was stirred at room temperature for 3 hours. After filtering the palladium carbon, the filtrate was concentrated under reduced pressure. The resulting residue was purified by silica gel chromatography [Silica Gel 60 (trade name, manufactured by Merck)] and eluted with n-hexane:ethyl acetate (2:1) to give Compound 4, or eluted with n-hexane:ethyl acetate (3:2) to give Compound 3. Furthermore, a fraction eluted with n-hexane:ethyl acetate (1:1) was concentrated under reduced pressure to give a crude purified product (936 mg). The resulting crude purified product was dissolved in a small amount of acetone and purified by preparative high performance liquid chromatography [column: YMC-Pack Pro C 18 AS-343 (20φ×250 mm), detection: UV absorption at 254 nm, flow rate: 10 ml/min] using acetonitrile-water (pH 3.5, acetic acid) (34:66) as the mobile phase to obtain three fractions. Each fraction was concentrated under reduced pressure to give Compound 32 (17.4 mg, 17 min), Compound 33 (49.4 mg, 18 min) and Compound 34 (347.4 mg, 21 min).

### EXAMPLE 18

### Compound 35

Radicicol (210 mg) was dissolved in pyridine (4 ml), and hydroxylamine hydrochloride (180 mg) was added thereto at room temperature, followed by heating 40° C. and stirring for 2.5 hours. After cooling to room temperature, water (100 ml) was added thereto and the mixture was extracted with ethyl acetate (80 ml). The ethyl acetate layer was dried over anhydrous sodium sulfate and then the ethyl acetate was distilled off under reduced pressure. The resulting residue was purified by preparative silica gel chromatography [Silica Gel 60 F254 (trade name, manufactured by Merck), 0.5 mm in thickness] using chloroform-methanol (90:10) as the developing solvent. A fraction showing an Rf value of 0.65 was collected, extracted with ethyl acetate and then concentrated under reduced pressure to give Compound 35 (40.0 mg).

### EXAMPLE 19

### Compounds 36, 37, 38 and 39

Radicicol (11.9 g) was dissolved in ethyl acetate (160 ml), and 5% palladium carbon (wet type) (300 mg) was added thereto, followed by hydrogen replacement (1 atm) and stirring at room temperature for 3 hours. After filtering the palladium carbon, the filtrate was concentrated under reduced pressure. The resulting residue was purified by silica gel chromatography [Silica Gel 60 (trade name, manufactured by Merck)] and eluted with n-hexane:ethyl acetate (2:1). The resulting fraction was concentrated under reduced pressure to give a fraction (1537 mg) comprising Compound 4 as the main component. This fraction was dissolved in methanol (21 ml)/tetrahydrofuran (7 ml), and cerium (III) chloride heptahydride (2130 mg) was added thereto, followed by stirring at room temperature for 30 minutes. Next, sodium boron hydride (790 mg) was slowly added to the solution under ice cooling, followed by stirring at room temperature for 10 minutes. The reaction solution was diluted with water (30 ml) and the pH value was adjusted to be neutral by adding a 1N aqueous hydrochloric acid solution, followed by further dilution with water (30 ml). The aqueous layer was extracted with ethyl acetate (100 ml×2). The resulting ethyl acetate layer was dried over anhydrous sodium sulfate and then the ethyl acetate was distilled off under reduced pressure to give a gummy substance (1.46 g). The obtained substance was dissolved in a solvent mixture of chloroform-methanol (4:1) and adsorbed by a silica gel [Silica gel 60 (trade name, manufactured by Merck)] column (400 ml) prepared by moistening with n-hexane. Then, elution was successively carried out with a solvent mixture of n-hexane-ethyl acetate. After eluting Compound 4 with n-hexane-ethyl acetate (2:1), the residue was eluted with n-hexane-ethyl acetate (3:2) to give each of fraction (1) (190 mg) and fraction (2) (398 mg) by concentration under reduced pressure. Fraction (1) was dissolved in a small amount of tetrahydrofuran and purified by preparative high performance liquid chromatography [column: YMC-Pack Pro C18 AS-343 (20φ×250 mm), detection: UV absorption at 254 nm, flow rate: 10 ml/min] using acetonitrile-water (pH 3.5, acetic acid) (35:65) as the mobile phase to obtain three fractions. Each of the fraction was concentrated under reduced pressure to give Compound 9 (87.2 mg) together with Compound 36 (11.3 mg, 20 min) and Compound 38 (10.1 mg, 25 min). Fraction (2) was dissolved in a small amount of tetrahydrofuran and purified by preparative high performance liquid chromatography under the same conditions as in fraction (1) to obtain two fractions. Each of the fractions was concentrated under reduced pressure to give Compound 37 (19.8 mg, 25 min) and Compound 39 (23.2 mg, 27 min).

### EXAMPLE 20

### Compounds 40 and 41

Compound 7 (61 mg) was dissolved in N,N-dimethylformamide (3 ml), and potassium hydroxide (5 mg) and 1-bromobutane (1 ml) were added thereto, followed by stirring at room temperature for 3.5 hours. After adding water (20 ml) to the reaction solution, the mixture was extracted with ethyl acetate (30 ml). The ethyl acetate layer was dried over anhydrous sodium sulfate and then the ethyl acetate was distilled off under reduced pressure. The resulting residue was dissolved in a small amount of acetone and purified by preparative high performance liquid chromatography [column: YMC-Pack Pro C18 AS-343 (20φ×250 mm), detection: UV absorption at 254 nm, flow rate: 10 ml/min] using acetonitrile-water (pH 3.5, acetic acid) under gradient of (60:40) to (90:10) as the mobile phase to obtain two fractions. Each of the fractions was concentrated under reduced pressure to give Compound 40 (25.6 mg, 33 min) and Compound 41 (21.4 mg, 40 min).

### EXAMPLE 21

### Compounds 42 and 43

Compound 7 (67 mg) was dissolved in N,N-dimethylformamide (3 ml), and potassium hydroxide (5 mg) and 1-bromohexane (1 ml) were added thereto, followed by stirring at room temperature for 3.5 hours. After adding water (20 ml) to the reaction solution, the mixture was extracted with ethyl acetate (30 ml). The ethyl acetate layer was dried over anhydrous sodium sulfate and then the ethyl acetate was distilled off under reduced pressure. The resulting residue was dissolved in a small amount of acetone and purified by preparative high performance liquid chromatography [column: YMC-Pack Pro C18 AS-343 (20φ×250 mm), detection: UV absorption at 254 nm, flow rate: 10 ml/min] using acetonitrile-water (pH 3.5, acetic acid) under gradient of (60:40) to (90:10) as the mobile phase to obtain two fractions. Each of the fractions was concentrated under reduced pressure to give Compound 42 (24.9 mg, 38 min) and Compound 43 (32.9 mg, 53 min).

### EXAMPLE 22

### Compound 44

Compound 7 (62 mg) was dissolved in N,N-dimethylformamide (3 ml), and potassium hydroxide (5 mg) and 1-bromopropane (1 ml) were added thereto, followed by stirring at room temperature for 1.5 hours. After adding water (20 ml) to the reaction solution, the mixture was extracted with ethyl acetate (30 ml). The ethyl acetate layer was dried over anhydrous sodium sulfate and then the ethyl acetate was distilled off under reduced pressure. The resulting residue was dissolved in a small amount of acetone and purified by preparative high performance liquid chromatography [column: YMC-Pack Pro C18 AS-343 (20φ×250 mm), detection: UV absorption at 254 nm, flow rate; 10 ml/min] using acetonitrile-water (pH 3.5, acetic acid) under gradient of (45:55) to (85:15) as the mobile phase. The obtained fraction was concentrated under reduced pressure to give Compound 44 (45.3 mg, 38 min).

### EXAMPLE 23

### Compound 45

Compound 7 (64 mg) was dissolved in N,N-dimethylformamide (3 ml), and potassium hydroxide (5 mg) and 2-(bromomethyl)cyclohexane (2 ml) were added thereto, followed by stirring at room temperature for 7 hours. After adding water (20 ml) to the reaction solution, the mixture was extracted with ethyl acetate (30 ml). The ethyl acetate layer was dried over anhydrous sodium sulfate and then the ethyl acetate was distilled off under reduced pressure. The resulting residue was dissolved in a small amount of acetone and purified by preparative high performance liquid chromatography [column: YMC-Pack Pro CIS AS-343 (20φ×250 mm), detection: UV absorption at 254 nm, flow rate: 10 ml/min] using acetonitrile-water (pH 3.5, acetic acid) under gradient of (65:35) to (85:15) as the mobile phase. The obtained fraction was concentrated under reduced pressure to give Compound 45 (24.7 mg, 70 min).

### EXAMPLE 24

### Compound 46

Compound 7 (200 mg) was dissolved in N,N-dimethylformamide (10 ml), and hydrobromic acid (5 ml) was added thereto, followed by stirring at 110° C. for 6 hours. After adding ethyl acetate (150 ml) to the reaction solution, the mixture was washed with sodium hydrogen carbonate (150 ml×2). The ethyl acetate layer was dried over anhydrous sodium sulfate and then the ethyl acetate was distilled off under reduced pressure. The resulting residue was dissolved in a small amount of methanol and purified by preparative high performance liquid chromatography [column: YMC-Pack Pro C18 AS-343 (20φ×250 mm), detection: UV absorption at 254 nm, flow rate; 10 ml/min] using acetonitrile-water (pH 3.5, acetic acid) under gradient of (38:62) to (50:50) as the mobile phase. The obtained fraction was concentrated under reduced pressure to give Compound 46 (17.0 mg, 18 min).

### EXAMPLE 25

### Compound 47

Compound 62 (154 mg) was dissolved in ethyl acetate (5 ml), and 5% palladium carbon (wet type) (30 mg) was added thereto, followed hydrogen replacement (1 atm) and stirring at room temperature for 90 minutes. After filtering the palladium carbon, the filtrate was distilled off under reduced pressure to give Compound 47 (134.8 mg).

### EXAMPLE 26

### Compound 48

Compound 61 (211 mg) was dissolved in ethyl acetate (11 ml), and 5% palladium carbon (wet type) (42 mg) was added thereto, followed by hydrogen replacement (1 atm) and stirring at room temperature for 90 minutes. After filtering the palladium carbon, the filtrate was distilled off under reduced pressure. The resulting residue (184 mg) was purified by preparative thin layer chromatography [Silica Gel 60 F254 (trade name, manufactured by Merck), 20 cm×20 cm, 0.5 mm in thickness] using chloroform-methanol (95:5) as the developing solvent. A part with an Rf value of 0.5 was collected, extracted with ethyl acetate and then concentrated under reduced pressure to give Compound 48 (159.7 mg).

### EXAMPLE 27

### Compounds 49 and 50

Compound 61 (233 mg) was dissolved in methanol (10 ml)/tetrahydrofuran (2 ml), and cerium (I) chloride heptahydride (754 mg) was added thereto, followed by stirring at room temperature for 30 minutes. Next, sodium boron hydride (236 mg) was slowly added to the solution under ice cooling, followed by stirring at room temperature for 5 minutes. After diluting the reaction solution with water (30 ml), a 1N aqueous hydrochloric acid solution (3 ml) was added to adjust the pH value to be neutral, and the mixture was further diluted with water (30 ml). The aqueous layer was extracted with ethyl acetate (100 ml×2). The resulting ethyl acetate layer was dried over anhydrous sodium sulfate and then the ethyl acetate was distilled off under reduced pressure to give a white powder (267 mg). Then, the obtained powder was dissolved in a small amount of water and purified by preparative high performance liquid chromatography [column: YMC-Pack Pro C18 AS-343 (20φ×250 mm), detection: UV absorption at 254 nm, flow rate: 10 ml/min] using acetonitrile-water (pH 3.5, acetic acid) (45:55) as the mobile phase to obtain two fractions. Each of the fractions was concentrated under reduced pressure to give Compound 49 (49.3 mg, 19 min) and Compound 50 (184.6 mg, 21 min).

### EXAMPLE 28

### Compound 51

Compound 62 (191 mg) was dissolved in methanol (10 ml)/tetrahydrofuran (4 ml), and cerium (III) chloride heptahydride (641 mg) was added thereto, followed by stirring at room temperature for 30 minutes. Next, sodium boron hydride (216 mg) was slowly added to the solution under ice cooling, followed by stirring at room temperature for 10 minutes. After diluting the reaction solution with water (30 ml), a 1N aqueous hydrochloric acid solution (3 ml) was added to adjust the pH value to be neutral, and the mixture was further diluted with water (30 ml). The aqueous layer was extracted with ethyl acetate (100 ml×2). The resulting ethyl acetate layer was dried over anhydrous sodium sulfate and then the ethyl acetate was distilled off under reduced pressure to give a colorless oily substance (182 mg). Then, the obtained oil was dissolved in a small amount of methanol and purified by preparative high performance liquid chromatography [column: YMC-Pack Pro C18 AS-343 (20φ×250 mm), detection: UV absorption at 254 nm, flow rate: 10 ml/min] using acetonitrile-water (pH 3.5, acetic acid) (30:70) as the mobile phase. The obtained fraction was concentrated under reduced pressure to give Compound 51 (137.3 mg, 29 min).

### EXAMPLE 29

### Compound 52

Radicicol (205 mg) was dissolved in acetone (10 ml), and S-chloroacetylthiophenol (330 mg) and potassium carbonate (310 mg) were added thereto, followed by stirring at room temperature for 1.5 hours. Then, water was added to the reaction solution, followed by extraction with ethyl acetate. After washing with a saturated aqueous sodium chloride solution and drying over anhydrous magnesium sulfate, the extract was dried under reduced pressure. Then, the residue was crudely purified by silica gel column chromatography (20 g, ethyl acetate:n-hexane=1:2). The resulting crude purified product was then purified by high performance liquid chromatography (20φ 250 mm, YMC-Pack Pro C18, eluted with water (to which acetic acid had been added, pH 3.5):acetonitrile=65:35) to give Compound 52 (105 mg).

### EXAMPLE 30

### Compound 53

Radicicol (406 mg) was dissolved in N,N-dimethylformamide (6 ml), and triethylamine (57.6 mg) and thiophenol (260 mg) were added thereto, followed by stirring under ice cooling overnight. Then, dilute hydrochloric acid was added to the reaction solution, followed by extraction with ethyl acetate. After washing successively with water and a saturated aqueous sodium chloride solution and drying over anhydrous magnesium sulfate, the extract was dried under reduced pressure. A 238 mg portion of the resulting residue was then purified by high performance liquid chromatography (20φ×250 mm, YMC-Pack Pro CIS, eluted with water (to which acetic acid had been added, pH 3.5):acetonitrile 65:35) to give Compound 53 (46.0 mg).

### EXAMPLE 31

### Compounds 54 and 55

Radicicol (378 mg) was dissolved in N,N-dimethylformamide (6 ml), and triethylamine (56.4 mg) and thioacetic acid (163 mg) were added thereto under ice cooling, followed by stirring under ice cooling overnight. Then, dilute hydrochloric acid was added to the reaction solution, followed by extraction with ethyl acetate. After washing successively with water and a saturated aqueous sodium chloride solution and drying over anhydrous magnesium sulfate, the extract was dried under reduced pressure. A 238 mg portion of the resulting residue was then purified by high performance liquid chromatography (20φ×250 mm, YMC-Pack Pro C18, eluted with water (to which acetic acid had been added, pH 3.5):acetonitrile=65:35) to give Compound 54 (49.0 mg) and Compound 55 (15.7 mg).

### EXAMPLE 32

### Compound 56

Radicicol (131 mg) was dissolved in acetone (10 ml), and methyl bromoacetate (219 mg) and potassium carbonate (183 mg) were added thereto, followed by stirring at room temperature for 3 hours. Then, dilute hydrochloric acid was added to the reaction solution, followed by extraction with ethyl acetate. After washing with a saturated aqueous sodium chloride solution and drying over anhydrous magnesium sulfate, the extract was dried under reduced pressure. Then, the resulting residue was recrystallized from ethyl acetate to give Compound 56 (81.8 mg).

### EXAMPLE 33

### Compound 57

Compound 7 (116 mg) was dissolved in acetone (10 ml), and methyl bromoacetate (330 mg) and potassium carbonate (276 mg) were added thereto, followed by stirring at room temperature for 3 days. Then, dilute hydrochloric acid was added to the reaction solution, followed by extraction with ethyl acetate. After washing with a saturated aqueous sodium chloride solution and drying over anhydrous magnesium sulfate, the extract was dried under reduced pressure. Then, the resulting residue was recrystallized from ethyl acetate to give Compound 57 (27.7 mg).

### EXAMPLE 34

### Compound 58

Radicicol (103 mg) was dissolved in N,N-dimethylformamide (6 ml), and nicotinic acid (190 mg), 1-ethyl-3-(3-dimethylaminopropyl)carbodiimide hydrochloride (270 mg) and 4-dimethylpyridine (37.7 mg) were added thereto, followed by stirring at room temperature for 3.5 hours. Then, water was added to the reaction solution, followed by extraction with ethyl acetate. After washing with a saturated aqueous sodium chloride solution and drying over anhydrous magnesium sulfate, the exact was dried under raked pressure. Then, the resulting residue was recrystallized from acetone-water to give Compound 58 (94.5 mg).

### EXAMPLE 35

### Compound 59

Compound 56 (312 mg) was dissolved in methanol (60 ml), and adding potassium hydroxide (206 mg)-water (20 ml) was added thereto, followed by stirring at room temperature for 15 minutes. Then, dilute hydrochloric acid was added to the reaction solution, followed by extraction with ethyl acetate. After washing with a saturated aqueous sodium chloride solution and drying over anhydrous magnesium sulfate, the extract was dried under reduced pressure. Then, the resulting residue was then purified by high performance liquid chromatography (20φ×250 mm, YMC-Pack Pro C18, eluted with water (to which acetic acid had been added, pH 3.5):acetonitrile=65:35) to give Compound 59 (10.6 mg).

### EXAMPLE 36

### Compounds 60, 61, 62, 63, 68 and 70

An aseptic liquid medium (pH 6) containing 2.0% glucose, 4.0% mannitol, 2.0% oatmeal, 0.4% yeast extract, 0.001% iron (II) sulfate heptahydrate, 0.001% zinc (II) sulfate heptahydrate, 0.001% manganese (II) sulfate tetra-pentahydrate and 0.0005% copper (II) sulfate pentahydrate was inoculated with Pochonia chlamydosporia var. chlamydosporia TE-0480 strain which was then cultured therein under shaking at 26° C. for 72 hours. Using a 50 L jar, an aseptic medium having the same composition as one employed in the seed culture was inoculated with 300 ml of the seed culture medium which was then cultured therein under aeration-agitation at 26° C. for 144 hours. After the completion of the culturing, the culture was centrifuged to thereby separate the cells from the supernatant. The supernatant was adsorbed by 1.5 L of EP-20 (manufactured by Mitsubishi Chemical), washed with water and then eluted with 3 L of methanol. The eluate was concentrated under reduced pressure and the obtained aqueous layer was extracted with ethyl acetate. The ethyl acetate layer was dehydrated over anhydrous sodium sulfate and concentrated under reduced pressure to give a brown syrupy substance (60 g).

A portion (30 g) of the culture extract was dissolved in a solvent mixture of chloroform-methanol (4:1) and adsorbed by a silica gel [Silica Gel 60 (trade name, manufactured by Merck)] column (1600 ml) prepared by moistening with n-hexane. Next, elution was successively carried out with n-hexane-ethyl acetate mixtures to give a fraction (130 mg) eluted with a solvent mixture of n-hexane-ethyl acetate (4:1), (2:1)-eluted fractions (1) (1.88 g) and (2) (780 mg), and a (1:2)-eluted fraction (3.56 g). The n-hexane-ethyl acetate (4:1)-eluted fraction (130 mg) was dissolved in a small amount of acetone and purified by preparative high performance liquid chromatography [column: YMC-Pack Pro C18 AS-343 (20φ×250 mm), detection: UV absorption at 254 nm, flow rate: 10 ml/min] using acetonitrile-water (pH 3.5, acetic acid) (55:45) as the mobile phase to obtain two fractions. Each of the fractions was concentrated under reduced pressure to give Compound 63 (23.5 mg, 20 min) and Compound 70 (4.8 mg, 24 min). The n-hexane-ethyl acetate (2:1)-eluted fraction (1.88 g) was recrystallized from acetone (30 ml) to give Compound 61 (277 mg). The eluted fraction (2) (780 mg) was purified by preparative thin layer chromatography [Silica Gel 60 F254 (trade name, manufactured by Merck), 20 cm×20 cm, 1 mm in thickness] using n-hexane-acetone (3:2) as the developing solvent. A part with an Rf value of 0.4 was collected, extracted with ethyl acetate and then concentrated under reduced pressure to give Compound 60 (80.3 mg). Next, the n-hexane-ethyl acetate (1:2)-eluted fraction (3.56 g) was dissolved in a solvent mixture of chloroform-methanol (4:1) and adsorbed by a silica gel [Silica Gel 60 (trade name, manufactured by Merck)] column (880 ml) prepared by moistening with chloroform. Next, elution was successively carried out with a solvent mixture of chloroform-methanol to give a chloroform-methanol (98:2)-eluted fraction (926 mg). Then, it was recrystallized from acetone-methanol (1 ml/3 ml) to give Compound 62 (339 mg). A chloroform-methanol (97:3)-eluted fraction (1.42 g) was purified by column chromatography using Sephadex LH-20 (trade name, manufactured by Amersham Pharmacia Biotech) (methanol, 900 ml), followed by preparative high performance liquid chromatography [column: YMC-Pack Pro C18 AS-343 (20φ×250 mm), detection: UV absorption at 254 nm, flow rate: 10 ml/min] using acetonitrile-water (pH 3.5, acetic acid) (30:70) as the mobile phase. The obtained fraction was concentrated under reduced pressure to give Compound 68 (4.5 mg, 21 min).

### EXAMPLE 37

### Compounds 64, 65, 71, 72 and 76

A portion (30 g) of a culture extract obtained as in Example 36 was dissolved in a solvent mixture of chloroform-methanol (4:1) and adsorbed by a silica gel (Silica Gel 60 (trade name, manufactured by Merck)) column (700 ml) prepared by moistening with chloroform. Next, elution was successively carried out with a solvent mixture of chloroform-methanol. A chloroform-methanol (93:7)-eluted fraction was divided into 2 portions, i.e., fractions (1) (156 mg) and (2) (164 mg). Fraction (1) was dissolved in a small amount of methanol and purified by preparative high performance liquid chromatography [column: YMC-Pack Pro C18 AS-343 (20φ×250 mm), detection: UV absorption at 254 nm, flow rate: 10 ml/min] using acetonitrile-water (pH 3.5, acetic acid) (30:70) as the mobile phase to obtain four fractions. Each of the fractions was concentrated under reduced pressure to give Compound 64 (35.4 mg, 21 min), Compound 72 (13.7 mg, 27 min), Compound 71 (6.6 mg, 29 ml) and Compound 65 (4.1 mg, 33 min). Fraction (2) was purified by preparative high performance liquid chromatography under the same conditions, and the obtained fractions were collected and concentrated under reduced pressure to give Compound 71 (3.7 mg) together with Compound 76 (4.5 mg, 20 min).

### EXAMPLE 38

### Compounds 69 and 74

A culture extract (39.5 g) obtained as in Example 36 was dissolved in n-hexane (300 ml×2). The precipitate thus formed was filtered and the solvent was removed. The obtained precipitate (8.1 g) was dissolved in a solvent mixture of chloroform-methanol (4:1) and adsorbed by a silica gel [Silica Gel 60 (trade name, manufactured by Merck)] column (650 ml) prepared by moistening with chloroform. Next, elution was successively carried out with a solvent mixture of chloroform-methanol to give a chloroform-methanol (88:12)-eluted fraction (318-mg). This fraction was divided in a small amount of methanol and purified by preparative high performance liquid chromatography [column: YMC-Pack Pro C18 AS-343 (20φ×250 mm), detection: UV absorption at 254 nm, flow rate; 10 ml/min] using acetonitrile-water (pH 3.5, acetic acid) (30:70) as the mobile phase to obtain two fractions. Each of the fractions was concentrated under reduced pressure to give Compound 74 (15.3 mg, 19 min) and Compound 69 (8.0 mg, 38 min).

### EXAMPLE 39

### Compound 66

A culture extract (39.5 g) obtained as in Example 36 was dissolved in n-hexane (300 ml×2). The precipitate thus formed was filtered and the solvent was removed. The obtained precipitate (8.1 g) was dissolved in a solvent mixture of chloroform-methanol (4:1) and adsorbed by a silica gel [Silica Gel 60 (trade name, manufactured by Merck)] column (650 ml) prepared by moistening with chloroform Next, elution was successively carried out with a solvent mixture of chloroform-methanol to give a chloroform-methanol (100:0)-eluted fraction (1.5 mg). Next, it was applied to silica gel column (620 ml) using a solvent mixture of n-hexane-ethyl acetate to give an n-hexane-ethyl acetate (2:1)-eluted fraction (321 mg). The obtained fraction was dissolved in a small amount of acetone and purified by preparative high performance liquid chromatography [column: YMC-Pack Pro C18 AS-343 (20φ×250 mm), detection: UV absorption at 254 nm, flow rate: 10 ml/min] using acetonitrile-water (pH 3.5, acetic acid) (under gradient of 45:55 to 70:30) as the mobile phase to give Compound 66 (14.4 mg, 34 min).

### EXAMPLE 40

### Compounds 73, 77 and 78

A culture extract (500 g) obtained as in Example 36 was dissolved in a solvent mixture of chloroform-methanol (4:1) and adsorbed by a silica gel [Silica Gel 60 (trade name, manufactured by Merck)] column (6000 ml) prepared by moistening with chloroform. Next, elution was successively carried out with a solvent mixture of chloroform-methanol. Thus, a chloroform-methanol (95:5)-eluted fraction (57 g) was obtained. A portion (18 g) of this fraction was further subjected to silica gel column (800 ml) using a solvent mixture of n-hexane-ethyl acetate. An n-hexane-ethyl acetate (2:1)-eluted fraction was divided into 2 portions, i.e., fractions (1) (126 mg) and (2) (985 mg). Fraction (1) was dissolved in a small amount of acetone and purified by preparative high performance liquid chromatography [column; YMC-Pack Pro C18 AS-343 (20φ×250 mm), detection: UV absorption at 254 nm, flow rate: 10 ml/min] using acetonitrile-water (pH 3.5, acetic acid) (55:45) as the mobile phase. The obtained fraction was concentrated under reduced pressure to give Compound 78 (13.4 mg, 26 min). On the other hand, fraction (2) was purified by preparative high performance liquid chromatography using acetonitrile-water (pH 3.5, acetic acid) (45:55) as the mobile phase to obtain the two fractions. Each of the fractions was concentrated under reduced pressure to give Compound 73 (38.1 mg, 10 min) and Compound 77 (67.2 mg, 26 min).

### EXAMPLE 41

### Compounds 67 and 75

A culture extract (500 g) obtained as in Example 36 was dissolved in a solvent mixture of chloroform-methanol (4:1) and adsorbed by a silica gel [Silica Gel 60 (trade name, manufactured by Merck)] column (6000 ml) prepared by moistening with chloroform. Next, elution was successively carried out with a solvent mixture of chloroform-methanol. Thus, a chloroform-methanol (95:5)-eluted fraction (57 g) was obtained. A portion (18 g) of this fraction was further subjected to silica gel column (800 ml) using a solvent mixture of n-hexane-ethyl acetate to obtain an n-hexane-ethyl acetate (1:2)-eluted fraction. This fraction was dissolved in a small amount of acetone and purified by preparative high performance liquid chromatography [column: YMC-Pack Pro C18 AS-343 (20φ×250 mm), detection: UV absorption at 254 nm, flow rate: 10 m/min] using acetonitrile-water (pH 3.5, acetic acid) (40:60) as the mobile phase to obtain two fractions. Each of the fractions was concentrated under reduced pressure to give Compound 67 (17.4 mg, 11 min) together with a semi-pure sample (23.9 mg). This semi-pure sample was purified by preparative thin layer chromatography [Silica Gel 60 F254 (trade name, manufactured by Merck), 20 cm×20 cm, 0.5 mm in thickness] using chloroform-methanol (90:10) as the developing solvent. A part with an Rf value of 0.5 was collected, extracted with ethyl acetate and then concentrated under reduced pressure to give Compound 75 (4.3 mg).

Table 2 shows the compounds synthesized and purified in the above Examples and data thereof.

| Comp. No. | Structure | 1H-NMR | 13C-NMR |
|---|---|---|---|
| 1 | 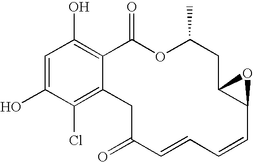 | (CDCl3/CD3OD, 500MHz)δppm: 1.48(d, J=6.7Hz, 3H), 1.83(ddd, J=15.3, 9.6, 3.7Hz, 1H), 2.33(dt, J=15.3, 3.7Hz, 1H), 2.94(dt, J=9.6, 3.0Hz, 1H), 3.19(s, 1H), 3.86(d, J=16.5Hz, 1H), 4.51(d, J=16.5Hz, 1H), 5.41(sxt, J=3.7Hz, 1H), 5.78(dd, J=9.8, 3.0Hz, 1H), 6.05(d, J=15.9Hz, 1H), 6.15(t, J=9.8Hz, 1H), 6.45(s, 1H), 7.48(dd, J=15.9, 9.8Hz, 1H) | (CDCl3/CD3OD, |
| #125MHz)δppm: 18.5(q), 36.3(t), 46.3(t), 55.8(d), 56.1(d), 71.2(d), 103.4(d), 108.0(s), 116.1(s), 130.1(d), 130.5(d), 135.0(d), 135.7(s), 139.6(d), 158.2(s), 161.2(s), 168.9(s), 199.1(s) | | | |
| 2 | 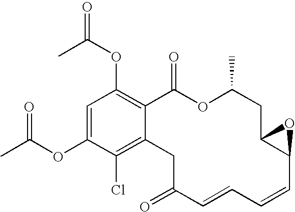 | (CDCl3, 500MHz)δppm: 1.51(d, J=6.7Hz, 3H), 1.54(ddd, J=14.6, 8.5, 3.7Hz, 1H), 2.24(s, 3H), 2.31(s, 3H), 2.38(dt, J=14.6, 3.7Hz, 1H), 2.99(dt, J=8.5, 3.7Hz, 1H), 3.49(qui, J=1.8Hz, 1H), 3.91(d, J=15.9Hz, 1H), 4.04(d, J=15.9Hz, 1H), 5.37(dq, J=6.7, 3.7Hz, 1H), 5.74(dd, J=11.0, 3.7Hz, 1H), 6.03(d, J=16.5Hz, 1H), 6.12(t, J=11.0Hz, 1H), 7.02(s, 1H), | |
| #7.45(dd, J=16.5, 11.0Hz, 1H) | (CDCl3, 125MHz)δppm: 18.6(q), 20.6(q), 20.7(q), 37.1(t), 45.1(t), 55.0(d), 55.4(d), 70.8(d), 117.8(d), 126.3(s), 126.5(s), 129.9(d), 130.6(d), 133.6(s), 136.1(d), 139.1(d), 146.6(s), 148.7(s), 163.8(s), 167.5(s), 168.1(s), 195.6(s) | | |
| 3 | 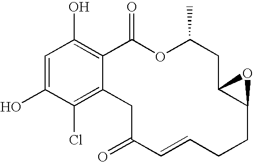 | (CDCl3/CD3OD, 500MHz)δppm: 1.21(m, 1H), 1.41(d, J=6.1Hz, 3H), 1.81(dt, J=15.9, 4.3Hz, 1H), 1.97(ddd, J=15.9, 4.9, 2.4Hz, 1H), 2.23(m, 1H), 2.35(ddd, J=14.0, 7.3, 2.4Hz, 1H), 2.48(dd, J=7.3, 3.7Hz, 1H), 2.52(dt, J=9.8, 2.4Hz, 1H), 2.74(sxt, J=2.4Hz, 1H), 4.27(d, J=18.3Hz, 1H), 4.40(d, J=18.3Hz, 1H), 5.21(dq, J=6.1, 4.3Hz, 1H), 6.05(dd, J=15.9, | |
| #1.2Hz, 1H), 6.48(s, 1H), 6.85(ddd, J=15.9, 11.0, 4.3Hz, 1H) | (CDCl3/CD3OD, 125MHz)δppm: 17.8(q), 29.2(t), 30.9(t), 38.4(t), 45.1(t), 56.0(d), 57.6(d), 72.1(d), 103.5(d), 106.3(s), 116.3(s), 130.1(d), 136.1(s), 148.0(d), 158.8(s), 163.4(s), 170.3(s), 197.0(s) | | |
| 4 | 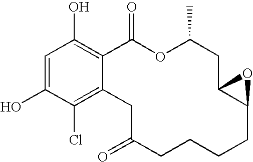 | (CDCl3/CD3OD, 500MHz)δppm: 1.02(m, 1H), 1.32(d, J=6.4Hz, 3H), 1.36(m, 1H), 1.41(m, 2H), 1.64(m, 1H), 1.77(ddd, J=15.5, 6.4, 2.7Hz, 1H), 1.92(ddd, J=14.0, 7.0, 3.0Hz, 1H), 2.04(ddd, J=15.5, 5.5, 4.3Hz, 1H), 2.34(m, 2H), 2.53(dt, J=8.8, 3.0Hz, 1H), 2.68(m, 1H), 4.02(d, J=18.3Hz, 1H), 4.25(d, J=18.3Hz, 1H), 5.15(d′qui, J=6.4, 2.7Hz, 1H), 6.37(s, 1H) | (CDCl3/CD3OD, |
| #125MHz)δppm: 18.6(q), 22.1(t), 23.2(t), 30.8(t), 36.1(t), 40.3(t), 46.5(t), 54.8(d), 57.6(d), 70.8(d), 103.1(d), 106.4(s), 115.6(s), 135.6(s), 158.2(s), 162.2(s), 169.6(s), 208.0(s) | | | |
| 5 | 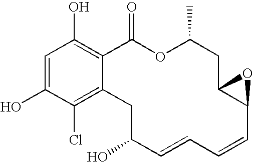 | (CD3OD, 500MHz)δppm: 1.41(d, J=6.7Hz, 3H), 1.95(dd, J=16.1, 5.5Hz, 1H), 2.50(ddd, J=16.1, 7.6, 3.0Hz, 1H), 2.97(qui, J=3.0Hz, 1H), 3.18(dd, J=12.8, 4.0Hz, 1H), 3.28(m, 1H), 3.73(dd, J=12.8, 10.4Hz, 1H), 4.52(sep, J=4.0Hz, 1H), 5.21(m, 1H), 5.31(dd, J=11.3, 5.5Hz, 1H), 5.78˜5.90(m, 3H), 6.35(s, 1H) | (CD3OD, 125MHz)δppm: 20.1(q), 36.0(t), 40.0(t), 56.0(d), |
| #57.2(d), 70.3(d), 72.7(d), 103.5(d), 108.6(s), 115.9(s), 127.4(d), 129.3(d), 130.7(d), 13.0(s), 139.1(d), 159.0(s), 162.8(s), 171.2(s) | | | |
| 7 | 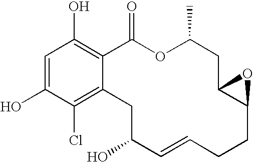 | (CD3OD, 500MHz)δppm: 1.19(m, 1H), 1.35(d, J=6.7Hz, 3H), 1.85(ddd, J=15.5, 5.5, 2.4Hz, 1H), 1.95(m, 1H), 2.01(m, 1H), 2.16(m, 1H), 2.27(ddd, J=15.6, 6.7, 3.0Hz, 1H), 2.50(dt, J=9.1, 3.0Hz, 1H), 2.85(qui, J=3.0Hz, 1H), 3.23(dd, J=12.5, 5.5Hz, 1H), 3.63(dd, J=12.5, 9.4Hz, 1H), 4.33(qui, J=5.5Hz, 1H), 5.07(d′qui, J=6.7, 2.4Hz, 1H), 5.28(dd, J=15.5, | |
| #5.5Hz, 1H), 5.38(ddd, J=15.5, 12.5, 5.5, 1H), 6.32(s, 1H) | (CD3OD, 125MHz)δppm: 20.2(q), 29.5(t), 31.3(t), 37.0(t), 40.2(t), 58.3(d), 59.6(d), 70.9(d), 71.8(d), 103.4(d), 109.3(d), 115.9(d), 129.1(d), 133.2(d), 140.0(d), 159.2(s), 163.0(s), 171.8(s) | | |
| 8 | 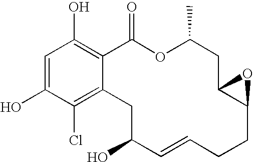 | (CD3OD, 500MHz)δppm: 1.27(m, 1H), 1.35(d, J=6.1Hz, 3H), 2.02(m, 1H), 2.03(m, 1H), 2.05(m, 1H), 2.17(m, 1H), 2.20(m, 1H), 2.86(dd, J=5.8, 3.0Hz, 1H), 2.92(dt, J=8.5, 3.0Hz, 1H), 3.25(dd, J=12.8, 5.5Hz, 1H), 3.30(dd, J=12.8, 9.8Hz, 1H), 4.43(m, 1H), 4.93(d′sxt, J=6.1, 1.8Hz, 1H), 5.37(dd, J=15.5, 4.0Hz, 1H), 5.64(dt, J=15.5, 6.1Hz, 1H), 6.41(s, 1H) | (CD3OD, |
| #125MHz)δppm: 20.4(q), 28.8(t), 31.2(t), 36.8(t), 39.7(t), 56.8(d), 57.6(d), 69.5(d), 70.6(d), 103.3(d), 114.7(s), 114.9(s), 129.7(d), 133.4(d), 138.5(s), 157.3(s), 158.2(s), 169.5(s) | | | |
| 9 | 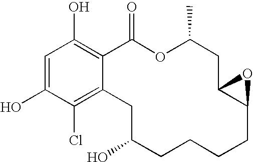 | (CD3OD, 500MHz)δppm: 1.11(m, 1H), 1.43(m, 1H), 1.45(d, J=6.4Hz, 3H), 1.48(m, 1H), 1.52(m, 1H), 1.57(m, 1H), 1.74(ddd, J=15.5, 7.3, 2.4Hz, 1H), 2.09(d′qui, J=14.3, 2.4Hz, 1H), 2.36(ddd, J=15.5, 7.3, 2.4Hz, 1H), 2.71(dt, J=9.4, 2.4Hz, 1H), 3.03(dt, J=7.3, 2.4Hz, 1H), 3.21(dd, J=7.3, 1.8Hz, 2H), 3.88(m, 1H), 5.20(d′qui, J=6.4, 2.4Hz, 1H), 6.38(s, 1H) | CD3OD, |
| #125MHz)δppm: 20.0(q), 25.2(t), 25.8(t), 32.2(t), 36.8(t), 38.0(t), 39.9(t), 58.2(d), 60.5(d), 71.4(d), 72.8(d), 103.0(d), 112.3(s), 115.3(s), 139.7(s), 158.0(s), 160.1(s), 171.1(s) | | | |
| 10 | 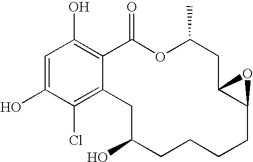 | (CD3OD, 500MHz)δppm: 1.29(m, 1H), 1.35(m, 2H), 1.38(m, 2H), 1.40(d, J=6.1Hz, 3H), 1.50(m, 1H), 1.57(m, 1H), 1.94(m, 1H), 2.37(m, 1H), 2.81(dt, J=7.3, 2.4Hz, 1H), 2.88(dt, J=7.3, 2.4Hz, 1H), 2.98(dd, J=13.4, 5.5Hz, 1H), 3.15(dd, J=13.4, 7.2Hz, 1H), 3.82(qui, J=6.1Hz, 1H), 5.12(d′sxt, J=6.1, 2.4Hz, 1H), 6.39(s, 1H) | (CD3OD, 125MHz)δppm: 20.5(q), 23.5(t), 24.6(t), |
| #30.2(t), 35.1(t), 37.8(t), 39.2(t), 57.1(d), 59.1(d), 72.2(d), 72.3(d), 102.9(d), 114.6(s), 115.9(s), 138.5(s), 156.6(s), 156.8(s), 169.8(s) | | | |
| 11 | 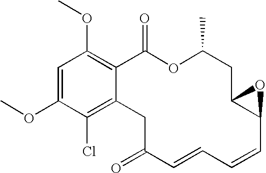 | (CDCl3, 500MHz)δppm: 1.50(d, J=6.7Hz, 3H), 1.61(ddd, J=14.6, 8.6, 3.7Hz, 1H), 2.40(dt, J=14.8, 3.7Hz, 1H), 3.02(dt, J=8.6, 1.8Hz, 1H), 3.40(br.t, J=1.8Hz, 1H), 3.75(d, J=15.9Hz, 1H), 3.83(s, 3H), 3.89(s, 3H), 3.95(d, J=15.9Hz, 1H), 5.35(dq, J=6.7, 3.7Hz, 1H), 5.69(dd, J=10.4, 4.3Hz, 1H), 6.07(d, J=15.9Hz, 1H), 6.12(t, J=10.4Hz, 1H), 6.45(s, 1H), | |
| #7.48(dd, J=15.9, 10.4Hz, 1H) | (CDCl3, 125MHz)δppm: 18.6(q), 37.3(t), 45.2(t), 55.5(d), 55.6(d), 56.3(q), 56.4(q), 70.2(d), 95.5(d), 115.5(s), 117.9(s), 130.2(d), 131.0(d), 132.4(s), 135.6(d), 138.6(d), 156.3(s), 156.9(s), 166.0(s), 196.4(s) | | |
| 12 | 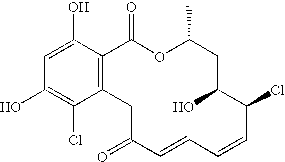 | (CD3OD, 500MHz)δppm: 1.42(d, J=6.4Hz, 3H), 1.89(ddd, J=15.2, 9.1, 3.6Hz, 1H), 2.03(dd, J=15.2, 7.0Hz, 1H), 3.66(d, J=16.2Hz, 1H), 3.99(dd, J=9.1, 5.8Hz, 1H), 4.21(d, J=16.2Hz, 1H), 5.12(dd, J=10.0, 5.8Hz, 1H), 5.38(dq, J=6.4, 3.6Hz, 1H), 5.75(t, J=10.0Hz, 1H), 5.95(d, J=16.1Hz, 1H), 6.17(t, J=11.0Hz, 1H), 6.46(s, 1H), 7.21(dd, J=16.1, 11.0Hz, | |
| #1H). | (CD3OD, 125MHz)δppm: 19.3(q), 38.2(t), 45.7(t), 60.6(d), 70.9(d), 72.6(d), 103.9(d), 114.3(s), 116.3(s), 130.8(d), 132.6(d), 134.6(s), 138.1(d), 141.1(d), 156.9(s), 157.0(s), 168.1(s), 199.8(s) | | |
| 13 | 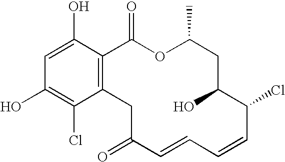 | (CD3OD, 500MHz)δppm: 1.46(d, J=6.1Hz, 3H), 1.96(ddd, J=14.0, 10.4, 3.0Hz, 1H), 2.12(ddd, J=14.0, 9.8, 3.0Hz, 1H), 3.76(d, J=15.3Hz, 1H), 3.88(dt, J=9.8, 3.0Hz, 1H), 4.73(d, J=15.3Hz, 1H), 5.00(dd, J=9.8, 3.0Hz, 1H), 5.27(m, 1H), 6.00(d, J=15.9Hz, 1H), 6.02(t, J=9.8Hz, 1H), 6.14(t, J=9.8Hz, 1H), 6.46(s, 1H), 7.18(dd, J=15.9, 9.8Hz, 1H) | (CD3OD, |
| #125MHz)δppm: 20.8(q), 40.6(t), 44.5(t), 60.4(d), 71.0(d), 72.1(d), 104.1(d), 113.8(s), 115.7(s), 127.6(d), 131.7(d), 135.4(s), 138.6(d), 139.1(d), 157.8(s), 158.6(s), 168.5(s), 198.7(s) | | | |
| 14 | 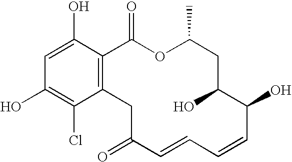 | (CD3OD, 500MHz)δppm: 1.42(d, J=6.7Hz, 3H), 1.87(ddd, J=15.3, 9.2, 3.7Hz, 1H), 1.95(dd, J=15.3, 6.7Hz, 1H), 3.63(d, J=15.9Hz, 1H), 3.80(m, 1H), 4.18(d, J=15.9Hz, 1H), 4.78(dd, J=8.5, 6.1Hz, 1H), 5.40(m, 1H), 5.73(dd, J=11.0, 8.5Hz, 1H), 5.90(d, J=15.9Hz, 1H), 6.14(t, J=11.0Hz, 1H), 6.41(s, 1H), 7.30(dd, J=15.9, 11.0Hz, 1H) | (CD3OD, 125MHz)δppm: 19.4(q), |
| #37.8(t), 45.8(t), 71.2(d), 71.9(d), 72.0(d), 103.8(d), 114.3(s), 116.6(s), 130.0(d), 131.5(d), 134.7(s), 142.4(d), 142.9(d), 156.7(s), 156.9(s), 168.4(s), 200.2(s) | | | |
| 15 | 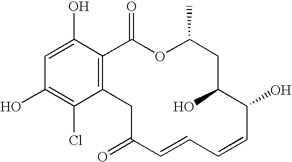 | (CD3OD, 500MHz)δppm: 1.44(d, J=6.1Hz, 3H), 1.83(ddd, J=14.6, 9.8, 3.0Hz, 1H), 2.10(ddd, J=14.6, 9.8, 3.0Hz, 1H), 3.64(dt, J=9.8, 3.0Hz, 1H), 3.87(d, J=15.9Hz, 1H), 4.53(d, J=15.9Hz, 1H), 4.56(m, 1H), 5.45(m, 1H), 5.94(dd, J=10.4, 7.3Hz, 1H), 5.96(d, J=15.9Hz, 1H), 6.12(t, J=10.4Hz, 1H), 6.44(s, 1H), 7.36(dd, J=15.9, 10.4Hz, 1H) | (CD3OD, 125MHz)δppm: |
| #21.0(q), 39.3(t), 44.5(t), 69.5(d), 71.2(d), 72.7(d), 104.0(d), 113.0(s), 115.9(s), 127.2(d), 131.3(d), 135.8(s), 140.6(d), 143.7(d), 158.2(s), 158.9(s), 168.7(s), 199.5(s) | | | |
| 16 | 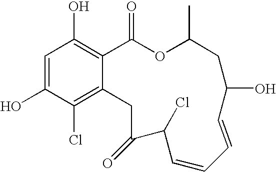 | (CD3OD, 500MHz)δppm: 1.29(d, J=6.1Hz, 3H), 1.70(ddd, J=11.6, 10.4, 2.4Hz, 1H), 2.17(dt, J=11.6, 6.1Hz, 1H), 4.01(d, J=18.9Hz, 1H), 4.05(m, 1H), 4.34(d, J=18.9Hz, 1H), 5.01(m, 1H), 5.46(d, J=9.8Hz, 1H), 5.82(dd, J=15.3, 9.8Hz, 1H), 6.02(t, J=11.0Hz, 1H), 6.10(dd, J=15.3, 11.0Hz, 1H), 6.30(t, J=11.0Hz, 1H), 6.45(s, 1H) | (CD3OD, 125MHz)δppm: 20.6(q), |
| #42.3(t), 42.4(t), 62.9(d), 70.4(d), 71.5(d), 103.8(d), 115.0(s), 116.4(s), 126.2(d), 127.6(d), 133.4(s), 134.8(d), 140.8(d), 158.5(s), 156.6(s), 169.4(s), 201.0(s) | | | |
| 17 | 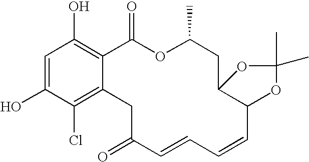 | (CD3OD, 500MHz)δppm: 1.38(s, 3H), 1.39(s, 3H), 1.46(d, J=6.4Hz, 3H), 1.87(dd, J=16.1, 9.1Hz, 1H), 2.08(ddd, J=16.1, 5.8, 1.8Hz, 1H), 3.73(dd, J=9.1, 5.8Hz, 1H), 3.85(d, J=16.4Hz, 1H), 4.38(d, J=16.4Hz, 1H), 4.71(t, J=9.1Hz, 1H), 5.65(m, 1H), 5.78(dd, J=11.3, 9.1Hz, 1H), 6.03(d, J=16.1Hz, 1H), 6.32(t, J=11.3Hz, 1H), 6.47(s, 1H), 7.83(dd, J=16.1, 11.3Hz, | |
| #1H) | (CD3OD, 125MHz)δppm: 21.8(q), 27.2(q), 27.3(q), 34.5(t), 45.9(t), 70.3(d), 75.9(d), 78.9(d), 103.8(d), 110.1(s), 111.0(s), 116.2(s), 130.3(d), 131.3(d), 136.6(s), 138.7(d), 142.2(d), 158.8(s), 160.3(s), 169.1(s), 200.4(s) | | |
| 18 | 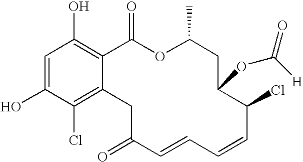 | (CDCl3/CD3OD, 500MHz)δppm: 1.52(d, J=6.4Hz, 3H), 2.02(ddd, J=14.9, 9.1, 4.0Hz, 1H), 2.09(dd, J=14.9, 8.4Hz, 1H), 3.79(d, J=6.1Hz, 1H), 4.20(d, J=16.1Hz, 1H), 5.23(dd, J=10.0, 5.8Hz, 1H), 5.34(dt, J=6.4, 4.0Hz, 1H), 5.39(dd, J=8.8, 5.8Hz, 1H), 5.73(t, J=10.0Hz, 1H), 6.00(d, J=16.1Hz, 1H), 6.16(t, J=11.0Hz, 1H), 6.48(s, 1H), 7.18(dd, J=16.1, 11.0Hz, | |
| #1H), 8.02(s, 1H) | (CDCl3/CD3OD, 125MHz)δppm: 19.0(q), 35.4(t), 45.4(t), 56.8(d), 70.0(d), 73.3(d), 103.6(d), 114.0(s), 115.1(s), 130.9(d), 133.1(d), 133.6(s), 135.6(d), 139.2(d), 156.3(s), 156.4(s), 160.6(d), 167.3(s), 198.8(s) | | |
| 19 | 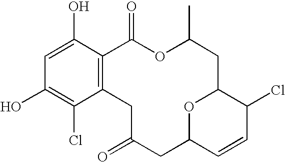 | (DMSO-d6, 500MHz)δppm: 1.25(d, J=6.1Hz, 3H), 1.74(ddd, J=15.3, 10.4, 7.9Hz, 1H), 2.01(d, J=15.3 Hz, 1H), 2.49(dd, J=14.6, 3.0Hz, 1H), 3.11(dd, J=14.6, 12.2Hz, 1H), 3.81(t, J=7.9Hz, 1H), 3.92(d, J=17.7Hz, 1H), 3.97(d, J=17.7Hz, 1H), 4.39(dd, J=7.9, 1.8Hz, 1H), 4.61(d, J=12.2Hz, 1H), 5.11(m, 1H), 5.83(dt, J=10.4, 1.8Hz, 1H), 5.91(dt, J=10.4, 1.8Hz, 1H), 6.47(s, | |
| #1H), 9.97(br.s, 1H), 10.35(br.s, 1H) | (DMSO-d6, 125MHz)δppm: 20.8(q), 37.8(t), 43.3(t), 46.0(t), 56.1(d), 69.2(d), 72.1(d), 72.6(d), 102.2(d), 112.3(s), 115.0(s), 126.9(d), 131.6(d), 131.8(s), 154.0(s), 154.7(s), 167.2(s), 202.8(s) | | |
| 20 | 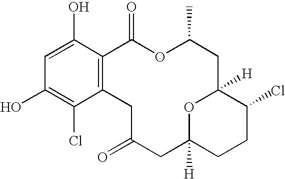 | (CDCl3, 500MHz)δppm: 1.40(d, J=6.4Hz, 3H), 1.77(ddd, J=10.4, 7.9, 1.5Hz, 1H), 1.91(m, 1H), 2.04(m, 1H), 2.13(m, 1H), 2.23(dd, J=12.5, 7.3Hz, 1H), 2.42(dd, J=14.6, 4.9Hz, 1H), 2.73(dd, J=15.5, 11.3Hz, 1H), 3.04(dd, J=14.6, 4.6Hz, 1H), 3.30(t, J=10.0Hz, 1H), 3.91(ddd, J=10.4, 7.9, 1.4Hz, 1H), 4.13(d, J=18.3Hz, 1H), 4.42(sxt, J=4.6Hz, 1H), 4.94(d, | |
| #J=18.3Hz, 1H), 5.26(sxt, J=6.4Hz, 1H), 6.60(s, 1H), 11.9(br.s, 1H) | (CDCl3, 125MHz)δppm: 20.6(q), 27.2(t), 28.7(t), 42.5(t), 45.3(t), 50.4(t), 58.6(d), 72.3(d), 78.1(d), 8.13(d), 103.8(d), 107.4(s), 114.5(s), 135.8(s), 156.3(s), 164.1(s), 170.3(s), 206.1(s) | | |
| 21 | 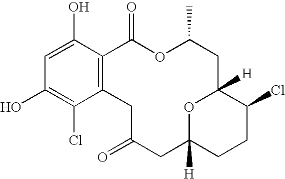 | (CDCl3/CD3OD, 500MHz)δppm: 1.20(d, J=6.1Hz, 3H), 1.41(m, 1H), 1.66(m, 1), 1.70(m, 1H), 1.75(m, 1H), 2.13(d, J=13.4Hz, 1H), 2.16(dd, J=11.0, 2.7Hz, 1H), 2.22(m, 1H), 2.57(t, J=11.0Hz, 1H), 3.28(t, J=9.8Hz, 1H), 3.42(ddd, J=11.6, 9.8, 4.6Hz, 1H), 3.51(dt, J=11.0, 2.7Hz, 1H), 4.13(d, J=19.2Hz, 1H), 5.05(d′sxt, J=6.1, 1.8Hz, 1H), 5.31(br.d, | |
| #J=19.2Hz, 1H), 6.33(s, 1H) | (CDCl3/CD3OD, 125MHz)δppm: 20.8(q), 32.4(t), 34.1(t), 37.9(t), 47.3(t), 47.9(t), 57.5(d), 74.4(d), 76.2(d), 81.7(d), 102.8(d), 107.6(s), 115.0(s), 134.7(s), 157.3(s), 161.3(s), 170.5(s), 207.0(s) | | |
| 22 | 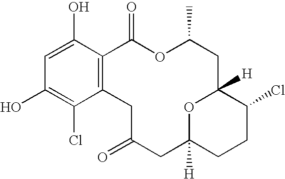 | (CDCl3/CD3OD, 500MHz)δppm: 1.30(d, J=6.1Hz, 3H), 1.75(dd, J=11.6, 5.5Hz, 1H), 1.80(d, J=14.6Hz, 1H), 1.99(m, 1H), 2.18(m, 2H), 2.54(br.s, 2H), 2.85(br.s, 2H), 3.82(m, 1H), 3.91(br.s, 1H), 4.42(br.s, 1H), 5.15(m, 1H), 6.43(s, 1H) | (CDCl3/CD3OD, 125MHz)δppm: 21.4(q), 27.0(t), 28.7(t), 35.8(t), 46.9(t), 46.9(t), 58.6(d), 68.2(d), 74.1(d), 77.0(d), 103.2(d), 107.0(s), |
| #115.5(s), 134.8(s), 158.0(s), 162.1(s), 170.9(s), 205.0(s) | | | |
| 23 | 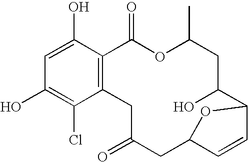 | (DMSO-d6, 500MHz)δppm: 1.26(d, J=6.7Hz, 3H), 1.44(ddd, J=14.6, 7.9, 3.0Hz, 1H), 2.16(d, J=12.8Hz, 1H), 2.37(dd, J=14.6, 5.5Hz, 1H), 2.76(dd, J=14.6, 4.9Hz, 1H), 3.20(m, 1H), 3.71(d, J=18.3Hz, 1H), 3.90(d, J=18.3Hz, 1H), 4.21(dt, J=9.8, 1.8Hz, 1H), 4.88(d, J=6.7Hz, 1H), 5.01(br.s, 1H), 5.20(sep, J=3.7Hz, 1H), 5.89(dt, J=6.1, 1.8Hz, | |
| #1H), 6.00(dt, J=6.1, 1.8Hz, 1H), 6.50(s, 1H), 9.97(s, 1H), 10.47(br.s, 1H) | DMSO-d6, 125MHz)δppm: 19.2(q), 37.6(t), 45.1(t), 48.3(t), 69.7(d), 70.3(d), 83.3(d), 88.4(d), 102.4(d), 111.7(s), 115.3(s), 129.3(d), 130.0(d), 130.2(s), 154.4(s), 154.7(s), 166.4(s), 203.8(s) | | |
| 25 | 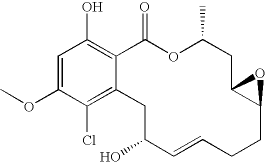 | (Acetone-d6, 500MHz)δppm: 1.22(dt, J=17.0, 9.1Hz, 1H), 1.43(d, J=6.7Hz, 3H), 1.98(m, 1H), 2.00(m, 1H), 2.06(m, 1H), 2.21(m, 1H), 2.33(ddd, J=15.8, 7.0, 3.0Hz, 1H), 2.50(dt, J=9.4, 1.8Hz, 1H), 2.83(m, 1H), 3.35(dd, J=12.5, 6.1Hz, 1H), 3.89(dd, J=12.5, 9.8Hz, 1H), 3.94(s, 3H), 4.43(m, 1H), 5.16(d-qui, J=6.7, 2.4Hz, 1H), 5.36(dd, J=15.2, 4.3Hz, | |
| #1H), 5.53(ddd, J=15.2, 6.7, 4.3, 1.8Hz, 1H), 6.58(s, 1H) | Acetone-d6, 125MHz)δppm: 20.2(q), 29.3(t), 31.1(t), 36.5(t), 40.3(t), 56.9(q), 57.2(d), 58.3(d), 70.6(d), 70.7(d), 100.2(d), 108.0(s), 116.4(s), 127.9(d), 133.4(d), 140.4(s), 160.4(s), 164.5(s), 171.8(s) | | |
| 26 | 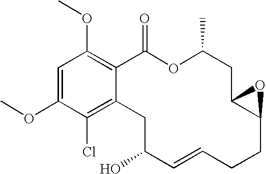 | (Acetone-d8,500MHz)δppm: 1.09(m, 1H), 1.40(ddd, J=14.6, 8.6, 3.0Hz, 1H), 1.48(d, J=6.7Hz, 3H), 2.10-2.20(m, 3H), 2.31(ddd, J=14.6, 6.7, 3.7Hz, 1H), 2.69(dt, J=9.2, 1.8Hz, 1H), 2.73(dd, J=13.4, 5.5Hz, 1H), 2.83(ddd, J=8.6, 3.7, 1.8Hz, 1H), 3.28(dd, J=13.4, 6.7Hz, 1H), 3.84(s, 3H), 3.94(s, 3H), 4.49(dd, J=13.4, 6.7Hz, 1H), 5.18(d-qui, J=6.7, | |
| #3.0Hz, 1H), 5.52(dd, J=15.9, 6.7Hz, 1H), 5.58(m, 1H), 6.75(s, 1H) | (Acetone-d6, 125MHz)δppm: 19.8(q), 28.7(t), 33.3(t), 39.0(t), 40.8(t), 56.1(d), 56.7(q), 56.8(q), 58.0(d), 71.5(d), 73.0(d), 96.5(d), 115.1(s), 119.9(s), 130.2(d), 135.7(d), 136.1(s), 156.9(s), 157.4(s), 167.2(s) | | |
| 27 | 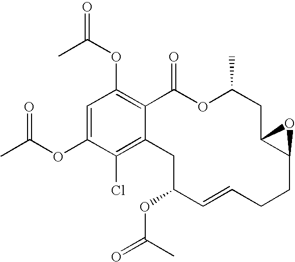 | (Acetone-d6, 500MHz)δppm: 1.15(m, 1H), 1.47(d, J=6.7Hz, 3H), 1.48(ddd, J=15.3, 7.9, 3.0Hz, 1H), 1.94(s, 3H), 2.13-2.23(m, 3H), 2.23(s, 3H), 2.30(ddd, J=15.3, 7.9, 4.3Hz, 1H), 2.34(s, 3H), 2.71(dt, J=9.2, 2.4Hz, 1H), 2.88(ddd, J=7.9, 3.7, 1.8Hz, 1H), 3.05(dd, J=14.0, 4.9Hz, 1H), 3.51(dd, J=14.0, 7.9Hz, 1H), 5.30(d-qui, J=6.7, 2.4Hz, 1H), 5.53(dd, | |
| #J=14.6, 7.3Hz, 1H), 5.55(m, 1H), 5.74(dt, J=146.6, 6.7Hz, 1H), 7.16(s, 1H) | (Acetone-d6, 125MHz)δppm: 19.8(q), 20.4(q), 20.6(q), 20.9(q), 28.0(t), 32.9(t), 37.4(t), 38.9(t), 56.2(d), 57.7(d), 72.8(d), 74.7(d), 118.6(d), 126.0(s), 129.0(s), 130.0(d), 134.1(d), 136.1(s), 147.6(s), 149.2(s), 165.5(s), 168.(s), 168.7(s), 168.4(s) | | |
| 28 | 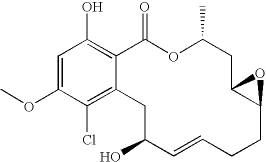 | (Acetone-d6, 500MHz)δppm: 1.17(d, J=6.1Hz, 3H), 1.53(dt, J=14.0, 5.5Hz, 1H), 1.62(m, 2H), 1.66(dd, J=14.0, 6.7Hz, 1H), 2.25(m, 2H), 2.68(dt, J=5.5, 2.4Hz, 1H), 2.80(m, 1H), 2.99(dd, J=17.1, 10.4Hz, 1H), 3.28(dd, J=17.1, 3.7Hz, 1H), 3.92(qui, J=6.1Hz, 1H), 3.98(s, 3H), 5.13(ddd, J=10.4, 3.7, 3.0Hz, 1H), 5.76(dd, J=15.3, 6.7Hz, 1H), 6.02(dt, J=15.3, | |
| #6.7Hz, 1H), 6.60(s, 1H) | (Acetone-d6, 125MHz)δppm: 23.9(q), 29.4(t), 31.5(t), 32.1(t), 42.3(t), 56.6(d), 57.1(q), 58.0(d), 65.8(d), 79.3(d), 99.9(d), 109.9(s), 112.0(s), 128.2(d), 135.8(d), 138.9(s), 162.1(s), 164(s), 170.0(s) | | |
| 29 | 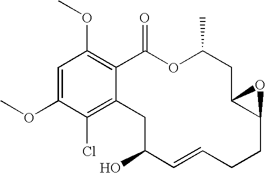 | (Acetone-d6, 500MHz)δppm: 1.22(m, 1H), 1.33(d, J=6.1Hz, 3H), 1.93(m, 1H), 1.96(m, 2H), 2.15(dd, J=9.8, 3.0Hz, 1H), 2.18(m, 1H), 2.76(dd, J=5.5, 3.7Hz, 1H), 2.84(dt, J=7.9, 2.4Hz, 1H), 3.09(dd, J=12.8, 10.4Hz, 1H), 3.18(dd, J=12.8, 4.3Hz, 1H), 3.88(s, 3H), 3.96(s, 3H), 4.52(m, 1H), 4.81(qui, J=6.1Hz, 1H), 5.32(ddd, J=16.5, 3.0, 1.2Hz, 1H), 5.72(m, 1H), | |
| #6.78(s, 1H) | Acetone-d6, 125MHz)δppm: 20.4(q), 28.6(t), 30.8(t), 36.7(t), 40.4(t), 55.3(d), 56.4(d), 56.7(q), 56.8(q), 68.6(d), 69.4(d), 96.8(d), 115.6(s), 118.9(s), 128.8(d), 133.2(d), 137.1(s), 158.0(s), 158.6(s), 167.7(s) | | |
| 30 | 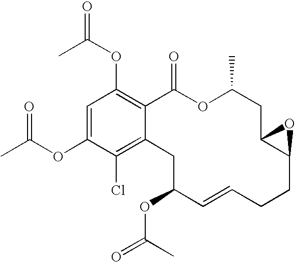 | (Acetone-d6, 500MHz)δppm: 1.27(m, 1H), 1.40(d, J=6.7Hz, 3H), 1.96(m, 1H), 1.98(s, 3H), 2.03-2.10(m, 3H), 2.11(m, 1H), 2.27(s, 3H), 2.34(s, 3H), 2.75(dt, J=7.9, 2.4Hz, 1H), 2.81(m, 1H), 3.35(dd, J=14.0, 6.7Hz, 1H), 3.55(dd, J=14.0, 8.5Hz, 1H), 5.09(d-qui, J=6.7, 2.4Hz, 1H), 5.42(dd, J=15.9, 4.9Hz, 1H), 5.57(dd, J=12.8, 6.7Hz, 1H), 5.69(ddt, J=15.9, | |
| #6.7, 1.2Hz, 1H), 7.18(s, 1H) | Acetone-d6, 125MHz)δppm: 19.8(q), 20.4(q), 20.9(q), 20.9(q), 27.5(t), 30.9(t), 38.3(t), 37.2(t), 55.4(d), 56.1(d), 71.2(d), 71.2(d), 119.2(d), 126.6(s), 128.1(s), 128.5(d), 133.2(d), 136.9(s), 148.7(s), 148.7(s), 165.2(s), 168.3(s), 169.0(s), 169.7(s) | | |
| 31 | 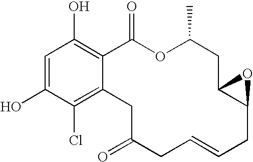 | (CDCl3/CD3OD, 500MHz)δppm: 1.31(d, J=6.7Hz, 3H), 1.66(m, 2H), 2.10(ddd, J=14.6, 4.9, 3.7, 3.0Hz, 1H), 2.53(ddd, J=14.0, 6.7, 3.7Hz, 1H), 2.50(dt, J=7.9, 3.0Hz, 1H), 2.69(dt, J=9.2, 3.0Hz, 1H), 2.81(dd, J=12.2, 7.3Hz, 1H), 3.14(dd, J=12.2, 7.9Hz, 1H), 4.06(d, J=18.3Hz, 1H), 4.22(d, J=18.3Hz, 1H), 5.18(m, 1H), 5.28(ddd, J=15.3, 7.3, | |
| #6.7Hz, 1H), 5.40(ddd, J=15.3, 7.9, 7.3Hz, 1H), 6.38(s, 1H) | CDCl3/CD3OD, 125MHz)δppm: 18.9(q), 33.8(t), 36.2(t), 45.4(t), 45.8(t), 53.7(d), 58.2(d), 71.5(d), 103.1(d), 107.6(s), 115.6(s), 125.9(d), 129.2(d), 135.2(s), 157.8(s), 161.0(s), 189.1(s), 205.6(s) | | |
| 32 | 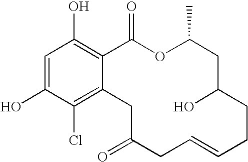 | (CDCl3, 500MHz)δppm: 1.40(d, J=6.4Hz, 3H), 1.63(m, 2H), 1.79(ddd, J=14.9, 6.4, 3.0Hz, 1H), 1.89(ddd, J=14.9, 7.6, 6.4Hz, 1H), 2.20(m, 2H), 3.04(dd, J=12.5, 7.6Hz, 1H), 3.16(dd, J=12.5, 7.6Hz, 1H), 3.78(ddd, J=6.7, 6.4, 5.2Hz, 1H), 4.48(d, J=18.3Hz, 1H), 4.59(d, J=18.3Hz, 1H), 5.33(d-qui, J=6.4, 3.0Hz, 1H), 5.47(dt, J=15.2, 7.6Hz, 1H), 5.60(ddd, | |
| #J=15.2, 7.6, 7.0Hz, 1H), 6.53(s, 1H) | (CDCl3, 125MHz)δppm: 20.6(q), 28.0(t), 35.3(t), 42.1(t), 45.1(t), 46.4(t), 66.4(d), 71.7(d), 103.7(d), 108.5(s), 115.2(s), 123.5(d), 135.2(d), 135.8(s), 156.0(s), 161.2(s), 168.0(s), 205.0(s) | | |
| 33 | 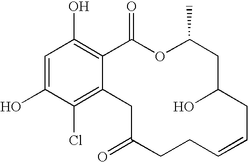 | (CDCl3, 500MHz)δppm: 1.42(d, J=6.4Hz, 3H), 1.86(m, 2H), 2.04(m, 1H), 2.20(m, 1H), 2.29(m, 1H), 2.32(m, 1H), 2.40(m, 1H), 2.73(ddd, J=13.4, 8.8, 3.0Hz, 1H), 3.60(ddd, J=6.7, 6.4, 5.8Hz, 1H), 4.24(d, J=17.7Hz, 1H), 4.38(d, J=17.7Hz, 1H), 5.41-5.50(m, 3H), 6.50(s, 1H) | (CDCl3, 125MHz)δppm: 20.3(q), 22.4(t), 34.6(t), 41.1(t), 41.4(t), 47.4(t), |
| #69.3(d), 71.2(d), 103.8(d), 107.8(s), 115.1(s), 126.5(d), 131.1(d), 136.1(s), 156.7(s), 161.9(s), 168.8(s), 207.8(s) | | | |
| 34 | 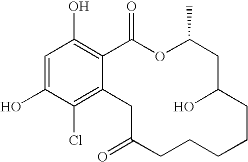 | (CDCl3, 500MHz)δppm: 1.24(m, 2H), 1.32(m, 1H), 1.44(d, J=6.7Hz, 3H), 1.46(m, 2H), 1.60(m, 2H), 1.69(m, 1H), 1.84(ddd, J=15.3, 6.7, 3.0Hz, 1H), 1.95(ddd, J=15.3, 7.3, 5.5Hz, 1H), 2.32(ddd, J=15.9, 9.2, 3.7Hz, 1H), 2.58(ddd, J=15.9, 8.5, 3.0Hz, 1H), 3.71(ddd, J=6.7, 6.1, 5.5Hz, 1H), 4.33(d, J=17.7Hz, 1H), 4.51(d, J=17.7Hz, 1H), 5.47(d-qui, 6.7, | |
| #2.4Hz, 1H), 6.54(s, 1H) | (CDCl3, 125MHz)δppm: 20.3(q), 22.0(t), 22.8(t), 25.2(t), 34.9(t), 40.6(t), 42.3(t), 46.5(t), 66.4(d), 71.4(d), 103.8(d), 107.9(s), 115.3(s), 136.2(s), 156.3(s), 161.7(s), 168.3(s), 207.9(s) | | |
| 35 | 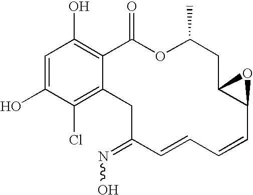 | (CD3OD, 500MHz)δppm: 1.52(d, J=6.7Hz, 3H), 1.57(m, 1H), 2.41(m, 1H), 3.01(ddd, J=9.2, 3.7, 1.8Hz, 1H), 3.34(m, 1H), 3.79(d, J=15.9Hz, 1H), 3.90(d, J=15.9Hz, 1H), 5.29(d-qui, J=6.7, 3.7Hz, 1H), 5.56(dd, J=10.4, 3.0Hz, 1H), 6.16(m, 1H), 6.43(s, 1H), 6.83(d, J=15.9Hz, 1H), 7.22(dd, J=15.9, 11.0Hz, 1H) | (CD3OD, 125MHz)δppm: 18.6(q), 36.2(t), 38.1(t), 56.6(d), |
| #57.0(d), 72.3(d), 103.4(d), 114.6(s), 116.2(s), 121.9(d), 130.8(d), 132.5(d), 132.8(d), 135.5(s), 154.8(s), 156.7(s), 157.1(s), 169.0(s) | | | |
| 36 | 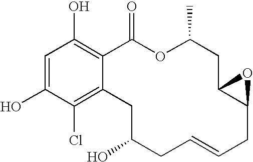 | (CDCl3/CD3OD, 500MHz)δppm: 1.29(d, J=6.7Hz, 3H), 1.37(ddd, J=14.6, 9.2, 2.4Hz, 1H), 2.01(m, 1H), 2.16(m, 2H), 2.19(m, 1H), 2.50(ddd, J=14.0, 7.3, 3.0Hz, 1H), 2.79(dd, J=14.0, 3.0Hz, 1H), 2.85(ddd, J=5.5, 3.0, 2.4Hz, 1H), 3.01(dt, J=9.2, 2.4Hz, 1H), 3.23(dd, J=14.0, 10.4Hz, 1H), 3.93(m, 1H), 5.22(d-sxt, J=6.7, 2.4Hz, 1H), 5.35(dt, J=15.3, 7.3Hz, | |
| #1H), 5.52(dt, J=15.3, 7.9Hz, 1H), 6.26(s, 1H) | (CDCl3/CD3OD, 125MHz)δppm: 19.3(q), 33.1(t), 36.4(t), 37.1(t), 39.4(t), 54.6(d), 59.4(d), 70.1(d), 70.5(d), 102.0(d), 111.8(s), 113.7(s), 127.6(d), 129.8(d), 138.1(s), 155.8(s), 157.7(s), 169.4(s) | | |
| 37 | 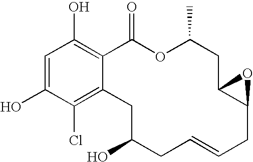 | (CDCl3/CD3OD, 500MHz)δppm: 1.22(d, J=6.7Hz, 3H), 1.27(ddd, J=14.0, 6.7, 1.8Hz, 1H), 1.90(dd, J=12.2, 11.6Hz, 1H), 2.07(ddd, J=14.0, 11.0, 3.0Hz, 1H), 2.40(m, 2H), 2.45(dd, J=14.6, 3.0Hz, 1H), 2.66(dd, J=14.0, 11.0Hz, 1H), 2.81(m, 1H), 2.88(q, J=2.4Hz, 1H), 4.05(m, 1H), 5.17(ddd, J=15.3, 9.8, 4.9Hz, 1H), 5.53(ddd, J=15.3, 10.4, 4.9Hz, | |
| #1H), 6.21(s, 1H) | (CDCl3/CD3OD, 125MHz)δppm: 20.1(q), 32.7(t), 37.0(t), 38.2(t), 38.8(t), 54.4(d), 59.4(d), 67.7(d), 69.9(d), 102.0(d), 112.7(s), 114.4(s), 127.5(d), 129.8(d), 136.1(s), 154.9(s), 155.1(s), 169.8(s) | | |
| 38 | 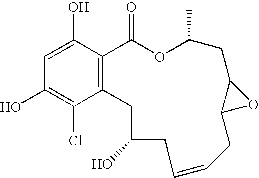 | (CDCl3/CD3OD, 500MHz)δppm: 1.44(d, J=6.1Hz, 3H), 1.84(dt, J=15.9, 4.9Hz, 1H), 1.96(m, 1H), 2.04(dt, J=15.9, 3.7Hz, 1H), 2.18(m, 1H), 2.41(ddd, J=14.0, 10.4, 3.7Hz, 1H), 2.55(ddd, J=14.0, 7.3, 2.4Hz, 1H), 2.74(dt, J=9.2, 2.4Hz, 1H), 2.96(m, 1H), 3.30(dd, J=13.4, 9.8Hz, 1H), 3.39(dd, J=13.4, 4.9Hz, 1H), 3.78(d-qui, J=6.1, 3.7Hz, 1H), | |
| #5.37(m, 1H), 5.52(dd, J=10.4, 7.9Hz, 1H), 5.70(ddd, J=10.4, 5.5, 4.9Hz, 1H), 6.38(s, 1H) | (CDCl3/CD3OD, 125MHz)δppm: 18.6(q), 30.1(t), 36.4(t), 36.6(t), 38.8(t), 55.4(d), 56.8(d), 71.2(d), 72.1(d), 102.6(d), 107.4(s), 115.6(s), 124.2(d), 130.7(d), 138.8(s), 157.3(s), 161.7(s), 170.2(s) | | |
| 39 | 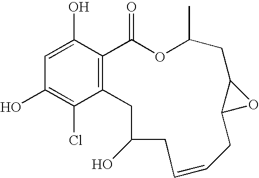 | (CDCl3/CD3OD, 500MHz)δppm: 1.08(d, J=6.1Hz, 3H), 1.52(dd, J=14.2, 6.7Hz, 1H), 1.57(ddd, J=14.2, 5.5, 4.9Hz, 1H), 2.18(t, like, J=4.9Hz, 2H), 2.42(m, 2H), 2.66(m, 1H), 2.68(m, 1H), 2.75(ddt, J=14.0, 5.5, 2.4Hz), 3.05(ddd, J=17.1, 9.2, 3.0Hz, 1H), 3.84(sxt, J=6.1Hz, 1H), 4.40(ddq, J=6.1, 5.5, 3.7Hz, 1H), 5.49(m, 2H), 6.28(s, 1H) | (CDCl3/CD3OD, |
| #125MHz)δppm: 22.7(q), 29.7(t), 34.6(t), 37.5(t), 40.5(t), 55.9(d), 57.4(d), 65.5(d), 77.7(d), 101.1(s), 102.0(d), 110.8(s), 126.4(d), 129.0(d), 137.9(s), 160.1(s), 162.0(s), 169.4(s) | | | |
| 40 | 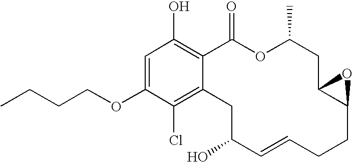 | (Acetone-d6, 500MHz)δppm: 0.98(t, J=7.3Hz, 3H), 1.21(m, 1H), 1.42(d, J=6.7Hz, 3H), 1.54(dt, J=15.3, 7.3Hz, 2H), 1.81(dt, J=15.3, 6.7Hz, 2H), 2.00(m, 2H), 2.05(m, 1H), 2.21(m, 1H), 2.34(ddd, J=15.9, 6.7, 3.0Hz, 1H), 2.50(dt, J=9.8, 1.8Hz, 1H), 2.85(m, 1H), 3.35(dd, J=12.8, 6.1Hz, 1H), 3.90(dd, J=12.8, 9.8Hz, 1H), 4.12(t, J=6.7Hz, 2H), 4.44(m, 1H), | |
| #5.15(d-qui, J=6.7, 2.4Hz, 1H), 5.35(dd, J=15.9, 4.3Hz, 1H), 5.53(ddd, J=15.9, 4.3, 1.8Hz, 1H), 6.55(s, 1H), 11.9(br.s, 1H) | Acetone-d6, 125MHz)δppm: 14.0(q), 19.8(t), 20.3(q), 29.3(t), 31.1(t), 31.7(t), 36.5(t), 40.3(t), 57.2(d), 58.3(d), 69.7(t), 70.5(d), 70.6(d), 100.8(d), 107.8(s), 116.7(s), 127.8(d), 133.4(d), 140.4(s), 159.9(s), 164.5(s), 171.8(s) | | |
| 41 | 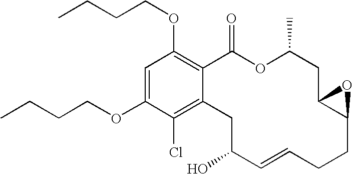 | (Acetone-d6, 500MHz)δppm: 0.92(t, J=7.3Hz, 3H), 0.97(t, J=7.3Hz, 3H), 1.07(m, 1H), 1.38(ddd, J=14.6, 9.2, 3.0Hz, 1H), 1.45(d, J=6.1Hz, 2H), 1.46(d, J=7.3Hz, 3H), 1.53(dt, J=15.3, 7.3Hz, 2H), 1.68(dt, J=14.6, 6.7Hz, 2H), 1.79(dt, J=15.3, 6.1Hz, 2H), 2.12-2.20(m, 3H), 2.33(ddd, J=14.6, 7.3, 3.7Hz, 1H), 2.71(m, 1H), 2.73(dd, J=9.2, 6.1Hz, | |
| #1H), 2.83(m, 1H), 3.29(dd, J=13.4, 7.3Hz, 1H), 4.04(t, J=6.1Hz, 2H), 4.12(t, J=6.1Hz, 2H), 4.48(qui, J=6.1Hz, 1H), 5.18(d-qui, J=7.3, 2.4Hz, 1H), 5.54(dd, J=15.9, 6.1Hz, 1H), 5.59(dd, J=15.9, 11.0Hz, 1H), 6.71(s, 1H) | (Acetone-d6, 125MHz)δppm: 14.0(q*2), 19.8(t*2), 19.9(q), 28.6(t), 31.9(t*2), 33.6(t), 39.1(t), 41.0(t), 56.1(d), 57.9(d), 69.3(t), 69.6(t), 71.5(d), | | |
| #73.3(d), 98.1(d), 115.3(s), 120.1(s), 130.2(d), 136.0(d), 136.0(s), 156.2(s), 156.8(s), 167.4(s) | | | |
| 42 | 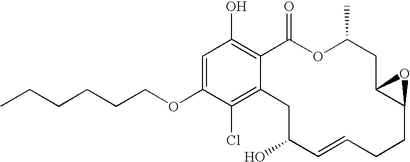 | (Acetone-d6, 500MHz)δppm: 0.90(t, J=7.3Hz, 3H), 1.22(m, 1H), 1.33-1.39(m, 4H), 1.42(d, J=6.7Hz, 3H), 1.52(m, 2H), 1.83(dt, J=15.3, 6.7Hz, 2H), 1.99(m, 2H), 2.05(m, 1H), 2.21(m, 1H), 2.33(ddd, J=16.5, 7.3, 3.7Hz, 1H), 2.50(dt, J=9.2, 1.8Hz, 1H), 2.83(m, 1H), 3.36(dd, J=12.2, 6.1Hz, 1H), 3.90(dd, J=12.2, 9.8Hz, 1H), 4.12(t, J=7.9Hz, 2H), | |
| #4.55(m, 1H), 5.15(d-qui, J=6.7, 1.8Hz, 1H), 5.35(dd, J=15.3, 3.7Hz, 1H), 5.53(ddd, J=15.3, 4.3, 1.8Hz, 1H), 6.55(s, 1H), 11.90(s, 1H) | (Acetone-d6, 125MHz)δppm: 14.2(q), 20.3(q), 23.2(t), 26.3(t), 29.3(t), 29.5(t), 31.1(t), 32.2(t), 36.5(t), 40.3(t), 57.2(d), 58.3(d), 70.0(t), 70.5(d), 70.6(d), 100.9(d), 107.8(s), 116.7(s), 127.8(d), 133.4(d), 140.4(s), 159.9(s), 164.5(s), | | |
| #171.8(s) | | | |
| 43 | 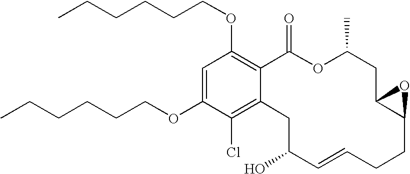 | (Acetone-d6, 500MHz)δppm: 0.88(t, J=7.3Hz, 3H), 0.90(t, J=7.3Hz, 3H), 1.07(m, 1H), 1.31(m, 4H), 1.35(m, 4H), 1.38(m, 1H), 1.43(m, 2H), 1.46(d, J=6.1Hz, 3H), 1.51(m, 2H), 1.69(dt, J=14.8, 6.7Hz, 2H), 1.81(dt, J=15.2, 6.1Hz, 2H), 2.11-2.21(m, 3H), 2.34(ddd, J=14.6, 7.3, 3.7Hz, 1H), 2.71(dd, J=7.3, 1.8Hz, 1H), 2.73(dd, J=13.4, 5.5Hz, 1H), 2.83(m, 1H), 3.29(dd, | |
| #J=13.4, 7.3Hz, 1H), 4.03(t, J=6.1Hz, 2H), 4.11(t, J=6.1Hz, 2H), 4.49(dd, J=12.5, 5.5Hz, 1H), 5.18(d-qui, J=6.1, 2.4Hz, 1H), 5.54(dd, J=15.9, 6.1Hz, 1H), 5.59(dd, J=15.9, 4.9Hz, 1H), 6.71(s, 1H) | (Acetone-d6, 125MHz)δppm: 14.2(q×2), 19.9(q), 23.2(t×2), 26.3(t), 26.4(t), 28.6(t), 29.8(t), 29.9(t), 32.2(t×2), 33.6(t), 39.1(t), 41.0(t), 56.1(d), 57.9(d), 69.6(t), | | |
| #69.9(t), 71.5(d), 73.3(d), 98.1(d), 115.3(s), 120.1(s), 130.2(d), 136.0(d), 136.0(s), 156.2(s), 156.8(s), 167.4(s) | | | |
| 44 | 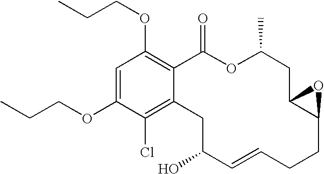 | (Acetone-d6, 500MHz)δppm: 0.97(t, J=7.3Hz, 3H), 1.05(t, J=7.3Hz, 3H), 1.08(m, 1H), 1.38(ddd, J=14.0, 8.5, 2.4Hz, 1H), 1.46(d, J=6.1Hz, 3H), 1.71(dt, J=13.4, 7.3Hz, 2H), 1.82(dt, J=13.4, 7.3Hz, 2H), 2.11-2.20(m, 3H), 2.33(ddd, J=14.6, 7.3, 3.6Hz, 1H), 2.71(dt, J=7.9, 2.4Hz, 1H), 2.73(dd, J=13.4, 5.5Hz, 1h), 2.82(ddd, J=8.5, 3.6, 2.4Hz, 1H), 3.30(dd, | |
| #J=13.4, 7.3Hz, 1H), 3.99(t, J=6.1Hz, 2H), 4.07(t, J=6.1Hz, 2H), 4.49(qui, J=6.1Hz, 1H), 5.18(d-qui, J=6.1, 2.4Hz, 1H), 5.54(dd, J=15.9, 6.1Hz, 1H), 5.59(dd, J=15.9, 5.5Hz, 1H), 6.70(s, 1H) | (Acetone-d6, 125MHz)δppm: 10.8(q×2), 19.9(q), 23.2(t×2), 28.6(t), 33.6(t), 39.1(t), 41.0(t), 56.1(d), 57.9(d), 71.1(t), 71.3(t), 71.5(d), 73.3(d), 98.1(d), 115.4(s), 120.1(s), 130.2(d), | | |
| #136.0(d), 136.0(s), 156.2(s), 156.7(s), 167.4(s) | | | |
| 45 | 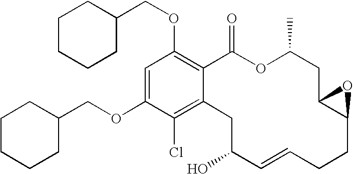 | (Acetone-d6, 500MHz)δppm: 1.06(m, 1H), 1.07-1.34(m, 10H), 1.39(ddd, J=14.6, 8.6, 2.4Hz, 1H), 1.47(d, J=6.1Hz, 3H), 1.63-1.85(m, 10H), 1.90(m, 2H), 2.11-2.21(m, 3H), 2.34(ddd, J=14.6, 7.9, 4.3Hz, 1H), 2.70(m, 1H), 2.73(dd, J=13.4, 5.5Hz, 1H), 2.83(ddd, J=8.6, 3.7, 1.8Hz, 1H), 3.30(dd, J=13.4, 7.3Hz, 1H), 3.84(d, J=6.1Hz, 2H), 3.91(d, J=6.7Hz, 2H), 4.49(dd, | |
| #J=12.5, 5.5Hz, 1H), 5.19(d-qui, J=6.1, 2.4Hz, 1H), 5.53(dd, J=15.9, 6.7Hz, 1H), 5.55(dd, J=15.9, 5.5Hz, 1H), 6.70(s, 1H) | (Acetone-d6, 125MHz)δppm: 20.0(q), 26.5(t×2), 27.2(t×2), 28.5(t), 30.2(t×2), 30.3(t×2), 30.4(t×2), 33.6(t), 38.6(d), 38.7(d), 39.1(t), 41.0(t), 56.2(d), 57.9(d), 71.6(d), 73.3(d), 74.8(t), 75.1(t), 97.9(d), 115.3(s), 119.9(s), 130.1(d), | | |
| #136.0(d), 136.0(s), 156.3(s), 156.9(s), 167.5(s) | | | |
| 46 | 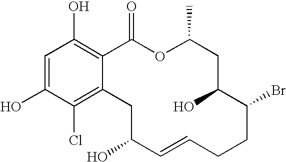 | (CD3OD, 500MHz)δppm: 1.38(d, J=6.7Hz, 3H), 1.92(ddd, J=14.6, 9.2, 3.7Hz, 1H), 2.05(m, 1H), 2.08(m, 1H), 2.18(ddd, J=14.6, 9.8, 3.0Hz, 1H), 2.28(m, 1H), 2.38(m, 1H), 3.30(m, 1H), 3.45(dd, J=12.2, 9.2Hz, 1H), 4.10(dd, J=9.2, 3.7Hz, 1H), 4.25(dd, J=9.2, 3.7Hz, 1H), 4.37(m, 1H), 5.30(m, 1H), 5.31(m, 1H), 5.61(ddd, J=15.9, 9.2, 6.7Hz, 1H), 6.43(s, 1H) | (CD3OD, 125MHz)δppm: 20.7(q), |
| #32.9(t), 32.2(t), 40.0(t), 42.9(t), 61.4(d), 67.8(d), 71.4(d), 71.6(d), 103.5(d), 111.5(s), 115.6(s), 129.6(d), 133.6(d), 138.3(s), 158.5(s), 160.8(s), 171.0(s) | | | |
| 47 | 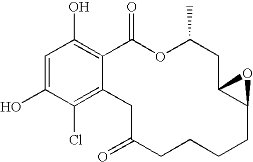 | (Acetone-d6, 500MHz)δppm: 1.08(m, 1H), 1.40(d, J=6.4Hz, 3H), 1.49(m, 2H), 1.52(m, 1H), 1.62(m, 1H), 1.87(ddd, J=15.5, 5.5, 2.4Hz, 1H), 1.96(m, 1H), 2.14(ddd, J=15.5, 6.4, 4.0Hz, 1H), 2.49(m, 2H), 2.59(dt, J=9.1, 2.4Hz, 1H), 2.78(ddd, J=5.5, 4.0, 2.4Hz, 1H), 3.70(d, J=17.4Hz, 1H), 4.32(d, J=17.4Hz, 1H), 5.21(d-qui, J=6.4, 2.4Hz, 1H), 6.24(d, J=2.4Hz, 1H), 6.28(d, | |
| #J=2.4Hz, 1H) | (Acetone-d6, 125MHz)δppm: 19.4(q), 22.8(t), 24.2(t), 31.8(t), 37.0(t), 40.8(t), 50.5(t), 55.4(d), 57.7(d), 71.2(d), 102.8(d), 108.4(s), 113.8(d), 140.7(s), 163.2(s), 166.1(s),171.5(s), 207.7(s) | | |
| 48 | 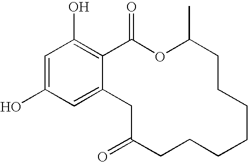 | (Acetone-d6, 500MHz)δppm: 1.31(m, 1H), 1.33(d, J=6.7Hz, 3H), 1.32-1.36(m, 7H), 1.43(m, 1H), 1.53(ddd, J=9.2, 7.3, 2.4Hz, 1H), 1.80(m, 1H), 1.85(m, 1H), 2.54(ddd, J=18.3, 5.5, 3.7Hz, 1H), 2.77(ddd, J=18.3, 11.0, 3.7Hz, 1H), 3.79(d, J=17.7Hz, 1H), 4.61(d, J=17.7Hz, 1H), 5.15(dq, J=6.7, 3.7Hz, 1H), 6.19(d, J=2.4Hz, 1H), 6.30(d, J=2.4Hz, 1H) | (Acetone-d6, 125MHz)δppm: |
| #21.2(q), 22.4(t), 23.3(t), 23.8(t), 24.6(t), 27.4(t), 35.2(t), 39.4(t), 50.5(t), 74.4(d), 102.6(d), 106.3(s), 113.8(d), 140.9(s), 163.0(s), 166.4(s), 172.2(s), 207.0(s) | | | |
| 49 | 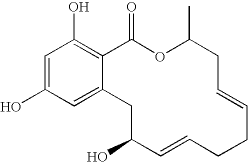 | (Acetone-d6, 500MHz)δppm: 1.37(d, J=6.1Hz, 3H), 2.02(m, 2H), 2.12(m, 2H), 2.29(ddd, J=14.0, 7.3, 6.1Hz, 1H), 2.56(ddd, J=14.0, 6.1, 3.7Hz, 1H), 3.23(dd, J=15.3, 5.5Hz, 1H), 3.25(dd, J=15.3, 5.5Hz, 1H), 4.5(q, J=6.1Hz, 1H), 5.23(d-qui, J=6.1, 3.7Hz, 1H), 5.27(dd, J=15.9, 6.1Hz, 1H), 5.28(dd, J=15.9, 8.5Hz, 1H), 5.39(dd, J=15.9, 6.1Hz, 1H), 5.43(dd, J=15.9, | |
| #6.7Hz, 1H), 6.24(d, J=2.4Hz, 1H), 6.53(d, J=2.4Hz, 1H) | (Acetone-d6, 125MHz)δppm: 19.5(q), 31.1(t), 32.2(t), 38.3(t), 42.4(t), 72.8(d), 73.4(d), 102.0(d), 107.4(s), 112.6(d), 127.0(d), 130.6(d), 133.3(d), 134.5(d), 144.4(s), 162.2(s), 164.6(s), 171.2(s) | | |
| 50 | 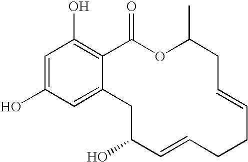 | (Acetone-d6, 500MHz)δppm: 1.39(d, J=6.7Hz, 3H), 1.94(m, 1H), 2.09(m, 1H), 2.12(m, 2H), 2.29(dt, J=15.3, 6.7Hz, 1H), 2.67(ddd,J=15.3, 7.3, 3.7Hz, 1H), 2.85(dd, J=13.4, 6.1Hz, 1H), 3.72(dd, J=13.4, 7.3Hz, 1H), 4.17(dd, J=11.0, 7.3Hz, 1H), 5.20(ddd, J=15.3, 6.1, 3.7Hz, 1H), 5.22(q, J=6.7Hz, 1H), 5.33(dd, J=15.3, 4.3Hz, 1H), 5.35(ddd, J=15.3, 11.0, | |
| #5.5Hz, 1H), 5.43(ddd, J=15.3, 9.2, 7.3Hz, 1H), 6.23(d, J=2.4Hz, 1H), 6.50(d, J=2.4Hz, 1H), 9.05(br.s, 1H), 11.87(br.s, 1H) | Acetone-d6, 125MHz)δppm: 19.5(q), 31.0(t), 31.9(t), 37.8(t), 43.4(t), 73.3(d), 75.0(d), 102.0(d), 105.9(s), 112.1(d), 126.4(d), 128.3(d), 133.8(d), 135.0(d), 145.5(s), 162.8(s), 166.2(s), 172.4(s) | | |
| 51 | 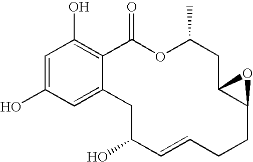 | (CD3OD, 500MHz)δppm: 1.23(ddt, J=14.0, 9.2, 3.7Hz, 1H), 1.40(d, J=6.1Hz, 3H), 2.05(m, 1H), 2.11(m, 2H), 2.13(m, 1H), 2.25(m, 1H), 2.57(dt, J=9.2, 2.4Hz, 1H), 2.74(dd, J=12.2, 6.7Hz, 1H), 2.91(dd, J=6.1, 2.4Hz, 1H), 3.77(dd, J=12.2, 7.9Hz, 1H), 4.13(dd, J=12.8, 6.7Hz, 1H), 5.16(d-qui, 6.1, 1.8Hz, 1H), 5.43(dd, J=15.9, 4.9Hz, 1H), 5.54(ddd, J=15.9, 4.9, | |
| #1.2Hz, 1H), 6.20(d, J=2.4Hz, 1H), 6.30(d, J=2.4Hz, 1H) | (CD3OD, 125MHz)δppm: 19.9(q), 29.4(t), 31.8(t), 36.9(t), 43.2(t), 58.2(d), 58.9(d), 70.5(d), 74.9(d), 102.5(d), 106.2(s), 113.3(d), 129.7(d), 133.3(d), 144.6(s), 163.5(s), 166.4(s), 172.6(s) | | |
| 52 | 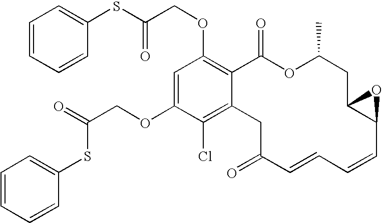 | (DMSO-d6, 500MHz)(rotational isomer of about 1:1)δppm: 1.39(d, J=6.7Hz, 1.5H), 1.41(d, J=6.7Hz, 1.5H), 1.50-1.57(m, 10H), 2.28(dt, J=14.6, 3.7Hz, 0.5H), 2.37(dt, J=14.0, 3.7Hz, 0.5H), 3.01(dt, J=7.9, 3.0Hz, 0.5H), 3.13(dt, J=8.6, 2.4Hz, 0.5H), 3.35(m, 0.6H), 3.38(m, 0.5H), 3.63(d, J=15.9Hz, 0.5H), 3.76(d, J=16.5Hz, 0.5H), 3.78(s, 4H), 3.85(d, J=15.9Hz, | |
| #0.5H), 3.97(d, J=16.5Hz, 0.5H), 5.17(m, 0.5H), 5.20(m, 0.5H), 5.70(dd, J=11.0, 4.9Hz, 0.5H), 5.74(dd, J=11.0, 3.7Hz, 0.5H), 6.04(d, J=15.9Hz, 0.5H), 6.09(d, J=16.5Hz, 0.5H), 6.22(t, J=11.6Hz, 0.5H), 6.25(t, J=10.4Hz, 0.5H), 6.54(s, 0.5H), 6.70(s, 0.5H), 7.18(m, 2H), 7.28-7.38(m, 7H), 7.42(d, J=7.9Hz, 2H) | (DMSO-d6, 125MHz)(rotational isoer of about 1:1)δppm: 18.1(q), 18.2(q), | | |
| #34.7(t), 34.9(t), 36.3(t), 36.4(t), 44.6(t), 44.9(t), 54.2(d×2), 55.0(d), 55.1(d), 89.6(d), 70.8(d), 109.4(d), 110.1(d), 119.4(s×2), 119.5(s×2), 125.8(d), 126.5(d), 127.7(d×2), 128.4(d×2), 129.0(d×2), 129.1(d×2), 129.6(d), 129.7(d), 130.5(d×2), 133.1(d), 134.5(s), 136.1(d), 136.2(d), 138.2(d), 138.4(d), 146.6(s), 146.7(s), | | | |
| #154,9(s), 155.3(s), 163.8(s×2), 166.1(s), 167.8(s), 170.5(s×2), 195.4(s), 195.8(s) | | | |
| 53 | 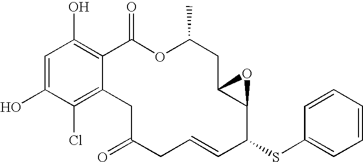 | (DMSO-d6, 500MHz)δppm: 1.22(d, J=6.7Hz, 3H), 1.31(ddd, J=14.0, 8.5, 2.4Hz, 1H), 2.24(ddd, J=14.0, 9.8, 3.7Hz, 1H), 2.94(ddd, J=8.5, 4.3, 2.4Hz, 1H), 2.99(d, J=7.9Hz, 2H), 3.05(dd, J=8.5, 2.4Hz, 1H), 3.61(t, J=8.5Hz, 1H), 3.84(d, J=1.8Hz, 2H), 5.08(dq, J=6.7, 2.4Hz, 1H), 5.55(dd, J=15.3, 8.5Hz, 1H), 5.84(dt, J=15.3, 7.9Hz, 1H), 6.48(s, 1H), 7.25(t, J=7.3Hz, | |
| #1H), 7.31(t, J=7.3Hz, 2H), 7.40(t, J=7.3Hz, 2H), 9.91(s, 1H), 10.39(s, 1H) | (DMSO-d6, 125MHz)δppm: 20.0(q), 37.3(t), 43.5(t), 45.1(t), 52.2(d), 54.6(d), 59.3(d), 70.3(d), 102.2(d), 112.0(s), 115.0(s), 126.9(d), 127.2(d), 128.9(d×2), 130.6(d), 131.2(d×2), 132.0(s), 133.5(s), 154.0(s), 154.6(s), 166.9(s), 203.1(s) | | |
| 54 | 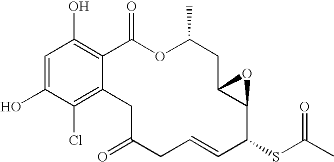 | (DMSO-d6, 500MHz)δppm: 1.22(d, J=6.1Hz, 3H), 1.30(ddd, J=14.6, 8.5, 2.3Hz, 1H), 2.23(ddd, J=14.6, 10.4, 3.7Hz, 1H), 2.35(s, 3H), 3.00(m, 2H), 3.07(dd, J=15.3, 7.3Hz, 1H), 3.11(dd, J=7.9, 1.8Hz, 1H), 3.76(t, J=7.9Hz, 1H), 3.87(s, 2H), 5.10(dq, J=6.1, 3.7Hz, 1H), 5.63(dd, J=15.3, 7.9Hz, 1H), 5.91(ddd, J=15.3, 7.9, 7.3Hz, 1H), 6.48(s, 1H), 9.91(s, | |
| #1H), 10.37(s, 1H) | (DMSO-d6, 125MHz)δppm: 20.1(q), 30.6(q), 37.3(t), 43.4(t), 45.2(t), 48.1(d), 55.3(d), 58.0(d), 70.2(d), 102.2(d), 112.1(s), 116.0(s), 127.2(d), 130.0(d), 132.0(s), 153.9(s), 154.6(s), 166.9(s), 193.6(s), 203.0(s) | | |
| 55 | 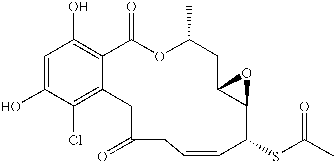 | (DMSO-d6, 500MHz)δppm: 1.22(d, J=5.5Hz, 3H), 1.30(m, 1H), 2.08(m, 1H), 2.38(s, 3H), 2.58(m, 1H), 3.06(br.s, 1H), 3.20(dd, J=18.3, 4.9Hz, 1H), 3.71(dd, J=18.3, 11.0Hz, 1H), 3.98(br.d, J=3.7Hz, 2H), 4.80(d, J=10.4Hz, 1H), 5.13(q like, J=5.5Hz, 1H), 5.17(dd, J=11.0, 10.4Hz, 1H), 5.67(dt, J=11.0, 4.9Hz, 1H), 6.49(s, 1H), 9.87(s, 1H), 10.39(s, 1H) | (DMSO-d6, |
| #125MHz)δppm: 20.3(q), 30.6(q), 37.8(t), 40.4(t), 40.8(d), 44.2(t), 52.9(d), 58.8(d), 70.2(d), 102.3(d), 112.0(s), 114.6(s), 126.1(d), 126.7(d), 132.5(s), 154.5(s), 154.7(s), 167.2(s), 193.9(s), 203.8(s) | | | |
| 56 | 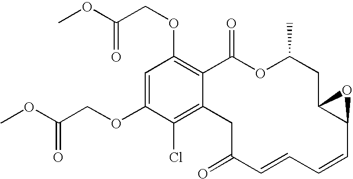 | (DMSO-d6, 500MHz)δppm: 1.10(d, J=6.7Hz, 3H), 1.15-1.24(m, 1H), 2.04(dt, J=14.0, 3.1Hz, 1H), 2.79(dt, J=6.1, 2.4Hz, 1H), 2.97(s, 1H), 3.10-3.20(m, 1H), 3.33(s, 3H), 3.35(s, 3H), 3.56(d, J=15.9Hz, 1H), 4.55(d, J=11.6Hz, 1H), 4.63(d, J=11.6Hz, 1H), 4.60-4.68(m, 1H), 4.86-4.92(m, 1H), 5.38(dd, J=10.4, 4.9Hz, 1H), 5.75(d, J=15.9Hz, 1H), 5.89(t, J=10.4Hz, 1H), | |
| #6.46(s, 1H), 7.00-7.06(m, 1H) | (DMSO-d6, 125MHz)δppm: 18.1(q), 36.4(t), 44.7(t), 51.8(q), 51.9(q), 54.8(d), 54.8(d), 65.3(t), 65.4(t), 70.4(d), 98.6(d), 114.6(s), 118.6(s), 129.7(d), 130.4(d), 131.7(s), 136.4(d), 138.3(d), 154.2(s), 154.6(s), 165.0(s), 168.3(s), 168.4(s), 195.0(s) | | |
| 57 | 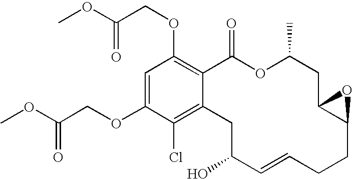 | (DMSO-d6, 500MHz)δppm: 0.66-0.73(m, 1H), 1.02(ddd, J=14.7, 9.2, 2.4Hz, 1H), 1.11(d, J=6.1Hz, 3H), 1.76-1.84(m, 3H), 1.98(ddd, J=1.7, 7.3, 4.3Hz, 1H), 2.28(dd, J=13.4, 6.1Hz, 1H), 2.41(d like, J=9.2Hz, 1H), 2.50(ddd, J=8.5, 3.7, 2.4Hz, 1H), 2.86(dd, J=13.4, 7.3Hz, 1H), 3.38(s, 3H), 3.42(s, 3H), 3.94-3.98(m, 1H), 4.52(s, 2H), 4.68(s, 1H), 4.78(ddd, J=13.4, | |
| #6.7, 2.4Hz, 1H), 5.08-5.18(m, 2H), 6.36(s, 1H) | (DMSO-d6, 125MHz)δppm: 19.1(q), 27.5(t), 32.1(t), 37.6(t), 39.0(t), 51.8(q), 51.9(q), 55.0(d), 56.8(d), 65.4(t), 65.5(t), 70.9(d), 71.5(d), 97.8(d), 114.5(s), 119.3(s), 128.9(d), 134.9(s), 135.2(d), 153.4(s), 154.2(s), 165.9(s), 168.4(s), 168.5(s). | | |
| 58 | 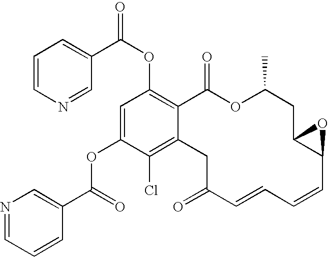 | (DMSO-d6, 500MHz)δppm: 0.86-0.95(m, 1H), 0.93(d, J=6.1Hz, 3H), 1.84(dt, J=14.7, 3.7Hz, 1H), 2.73(dt, J=7.9, 3.1Hz, 1H), 2.97(s, 1H), 3.60(d, J=15.9Hz, 1H), 3.87(d, J=15.9Hz, 1H), 4.76-4.83(m, 1H), 5.44(dd, J=10.4, 4.3Hz, 1H), 4.78(d, J=15.9Hz, 1H), 5.94(t, J=10.4Hz, 1H), 7.00(dd, J=15.9, 10.4Hz, 1H), 7.35(dt, J=7.9, 4.9Hz, 2H), 7.54(s, 1H), 8.10(dt, | |
| #J=7.9, 1.8Hz, 1H), 8.17(dt, J=7.9, 1.8Hz, 1H), 8.60(dt, J=4.3, 1.2Hz, 2H), 8.88(d, J=2.4Hz, 1H), 8.94(d, J=2.4Hz, 1H) | (DMSO-d6, 125MHz)δppm: 18.1(q), 36.2(t), 44.5(t), 54.3(d), 54.8(d), 71.7(d), 119.0(d), 123.8(s), 124.0(s), 124.3(d), 124.4(d), 126.0(s), 127.3(s), 129.6(d), 130.4(d), 133.6(s), 136.5(d), 137.6(d), 137.8(d), 138.5(d), 146.5(s), 148.1(s), | | |
| #150.5(d), 150.7(d), 154.8(d), 154.9(d), 162.2(s), 163.0(s), 163.2(s), 194.7(s) | | | |
| 59 | 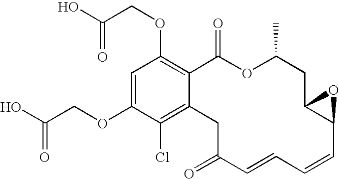 | (DMSO-d6, 500MHz)δppm: 0.93(d, J=6.7Hz, 3H), 1.42-1.50(m, 1H), 2.23-2.40(m, 1H), 2.79-2.84(m, 1H), 2.94(s, 1H), 3.26-3.33(m, 2H), 4.32(s, 2H), 4.48(s, 2H), 4.86-4.94(m, 1H), 5.34-5.46(m, 1H), 5.65(d, J=16.5Hz, 1H), 6.06(t, J=10.4Hz, 1H), 6.37(s, 1H), 6.70(dd, J=16.5, 11.0Hz, 1H), 12.60(brs, 2H) | (DMSO-d6, 125MHz)δppm: 19.7(q), 36.0(t), 44.1(t), 59.6(d), 59.7(d), |
| #65.7(t), 65.9(t), 70.8(d), 99.8(d), 115.1(s), 118.6(s), 130.1(d), 130.8(s), 131.7(d), 132.0(d), 132.8(d), 154.1(s), 166.2(s), 169.0(s), 169.1(s), 169.3(s), 199.0(s). | | | |
| 60 | 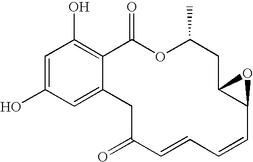 | (Acetone-d6, 500MHz)δppm: 1.60(d, J=6.7Hz, 3H), 1.85(ddd, J=14.9, 8.5, 3.7Hz, 1H), 2.44(dt, J=14.9, 3.7Hz, 1H), 3.12(dt, J=8.5, 2.7Hz, 1H), 3.32(qui, J=2.7Hz, 1H), 3.56(d, J=14.0Hz, 1H), 4.97(d, J=14.0Hz, 1H), 5.83(dd, J=10.7, 2.7Hz, 1H), 5.97(d, J=16.1Hz, 1H), 6.28(d, J=2.7Hz, 1H), 6.29(d, J=2.7Hz, 1H), 6.30(ddd, J=11.3, 10.7, 1.5Hz, 1H), 7.83(ddd, | |
| #J=16.1, 11.3, 1.5Hz, 1H), 9.16(br.s, 1H), 10.94(br.s, 1H) | (Acetone-d6, 125MHz)δppm: 18.9(q), 36.9(t), 43.9(t), 55.7(d), 56.0(d), 72.0(d), 102.8(d), 106.0(s), 110.1(d), 130.6(d), 131.7(d), 137.2(d), 140.3(s), 141.6(d), 162.9(s), 165.5(s), 170.4(s), 198.9(s) | | |
| 61 | 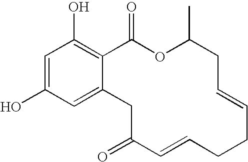 | (Acetone-d6, 500MHz)δppm: 1.23(d, J=6.4Hz, 3H), 2.04(m, 1H), 2.12(m, 1H), 2.12-2.25(m, 3H), 2.39(dt, J=10.4, 3.0Hz, 1H), 3.50(d, J=15.2Hz, 1H), 3.90(d, J=15.2Hz, 1H), 5.09(d-sxt, J=6.4, 3.0Hz, 1H), 5.29(ddd, J=15.2, 7.0, 4.3Hz, 1H), 5.31(ddd, J=15.2, 7.6, 4.9Hz), 1H), 5.86(d, J=16.1Hz, 1H), 6.09(d, J=2.4Hz, 1H), 6.20(d, J=2.4Hz, 1H), 6.64(ddd, J=16.1, | |
| #7.6, 6.4Hz, 1H), 9.92(br.s, 1H), 10.27(br.s, 1H) | (Acetone-d6, 125MHz)δppm: 19.2(q), 30.3(t), 30.4(t), 37.9(t), 45.2(t), 71.0(d), 101.4(d), 108.8(d), 110.3(s), 128.3(d), 129.7(d), 131.2(d), 136.6(s), 148.2(d), 159.5(s), 160.2(s), 168.4(s), 196.6(s) | | |
| 62 | 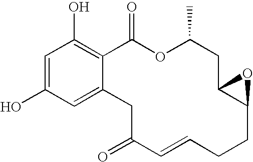 | (Acetone-d6, 500MHz)δppm: 1.19(m, 1H), 1.37(d, J=6.7Hz, 3H), 1.63(ddd, J=16.1, 5.2, 4.0Hz, 1H), 2.06(dt, J=16.1, 4.0Hz, 1H), 2.27(m, 1H), 2.29(m, 1H), 2.47(dd, J=8.8, 4.6Hz, 1H), 2.51(dt, J=9.4, 2.7Hz, 1H), 2.80(ddd, J=5.5, 4.0, 2.7Hz, 1H), 3.65(d, J=17.7Hz, 1H), 4.65(d, J=17.7Hz, 1H), 5.18(m, 1H), 6.02(d, J=15.8Hz, 1H), 6.24(d, J=2.4Hz, 1H), 6.27(d, J=2.4Hz, | |
| #1H), 6.91(ddd, J=15.8, 11.0, 4.6Hz, 1H), 9.23(br.s, 1H), 11.91(br.s, 1H) | (Acetone-d6, 125MHz)δppm: 18.1(q), 29.8(t), 31.8(t), 37.0(t), 48.2(t), 55.7(d), 57.0(d), 72.5(d), 102.8(d), 105.9(s), 113.9(d), 131.9(d), 141.3(s), 148.6(d), 163.4(s), 167.1(s), 171.9(s), 196.9(s) | | |
| 63 | 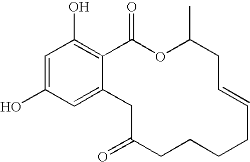 | (Acetone-d6, 500MHz)δppm: 1.35(d, J=6.7Hz, 3H), 1.50(m, 2H), 1.59(m, 2H), 2.03(m, 1H), 2.10(m, 1H), 2.30(dt, J=14.0, 5.5Hz, 1H), 2.49(m, 2H), 2.56(ddd, J=14.0, 3.7, 3.0Hz, 1H), 3.56(d, J=17.1Hz, 1H), 4.36(d, J=17.1Hz, 1H), 5.28(d-qui, J=6.7, 3.7Hz, 1H), 5.45(dd, J=15.9, 5.5Hz, 1H), 5.48(dd, J=15.9, 5.5Hz, 1H), 6.24(d, J=2.4Hz, 1H), 6.31(d, J=2.4Hz, 1H), 9.19(br.s, | |
| #1H), 11.76(br.s, 1H) | (Acetone-d6, 125MHz)δppm: 19.1(q), 22.9(t), 26.0(t), 33.0(t), 38.1(t), 41.1(t), 50.4(t), 73.5(d), 102.7(d), 106.2(s), 113.2(d), 125.8(d), 135.2(d), 140.9(s), 163.1(s), 166.4(s), 171.8(s), 207.3(s) | | |
| 64 | 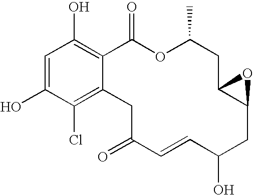 | (CD3OD, 500MHz)δppm: 1.26(dd, J=12.2, 9.2Hz, 1H), 1.44(d, J=6.1Hz, 3H), 1.76(d, J=15.9Hz, 1H), 2.54(d, J=12.2Hz, 1H), 2.61(d, J=9.2Hz, 1H), 2.87(br.s, 1H), 4.36(d, J=18.3Hz, 1H), 4.36(m, 1H), 4.53(d, J=18.3Hz, 1H), 5.21(br.s, 1H), 6.14(d, J=15.9Hz, 1H), 6.46(s, 1H), 6.82(dd, J=15.9, 9.2Hz, 1H) | (CD3OD, 125MHz)δppm: 18.0(q), 37.4(t), 41.2(t), 45.7(t), 55.4(d), |
| #56.8(d), 70.5(d), 73.4(d), 104.3(d), 107.8(s), 116.8(s), 129.4(d), 137.2(s), 150.6(d), 160.0(s), 164.2(s), 171.3(s), 198.5(s) | | | |
| 65 | 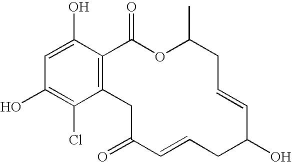 | (CD3OD, 500MHz)δppm: 1.19(d, J=6.4Hz, 3H), 2.16(ddd, J=14.3, 7.6, 6.7Hz, 1H), 2.25(dt, J=12.8, 8.2Hz, 1H), 2.36(ddd, J=12.8, 7.3, 3.4Hz, 1H), 2.47(ddd, J=14.3, 6.7, 3.7Hz, 1H), 4.10(d, J=17.7Hz, 1H), 4.14(d, J=17.7Hz, 1H), 4.24(m, 1H), 5.20(m, 1H), 5.23(dd, J=15.5, 6.1Hz, 1H), 5.38(dt, J=15.5, 7.6Hz, 1H), 5.80(d, J=15.8Hz, 1H), 6.35(s, 1H), 6.69(dt, J=15.8, 8.2Hz, | |
| #1H) | (CD3OD, 125MHz)δppm: 18.9(q), 38.0(t), 40.8(t), 45.8(t), 72.6(d), 73.5(d), 104.1(d), 109.6(s), 116.7(s), 126.3(d), 132.1(d), 136.6(s), 137.1(d), 146.1(d), 159.9(s), 162.3(s), 170.7(s), 198.7(s) | | |
| 66 | 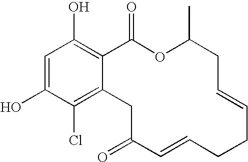 | (Acetone-d6, 500MHz)δppm: 1.28(d, J=6.4Hz, 3H), 2.15(m, 2H), 2.24(m, 1H), 2.28(m, 2H), 2.57(ddd, J=14.6, 8.2, 4.3Hz, 1H), 4.10(d, J=17.7Hz, 1H), 4.33(d, J=17.7Hz, 1H), 5.23(dd like, J=15.2, 7.3Hz, 1H), 5.36(ddd, J=15.2, 8.8, 5.8Hz, 1H), 5.37(dq, J=6.4, 3.7Hz, 1H), 5.81(d, J=15.5Hz, 1H), 6.57(s, 1H), 6.65(dt, J=15.5, 7.3Hz, 1H), 9.85(br.s, 1H), 11.46(s, 1H) | (Acetone-d6, |
| #125MHz)δppm: 18.1(q), 31.8(t×2), 37.3(t), 46.2(t), 73.4(d), 103.8(d), 108.4(s), 116.1(s), 127.7(d), 131.0(d), 133.0(d), 138.0(s), 147.3(d), 158.8(s), 163.5(s), 170.7(s), 195.2(s) | | | |
| 67 | 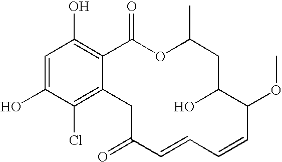 | (CD3OD, 500MHz)δppm: 1.42(d, J=6.4Hz, 3H), 1.85(m, 2H), 3.12(s, 3H), 3.70(d, J=16.4Hz, 1H), 3.88(m, 1H), 4.20(d, J=16.4Hz, 1H), 4.41(dd, J=7.9, 4.6Hz, 1H), 5.36(sxt, J=6.4Hz, 1H), 5.70(dd, J=10.0, 9.7Hz, 1H), 5.90(d, J=16.4Hz, 1H), 6.29(dd, J=11.3, 10.0Hz, 1H), 6.43(s, 1H), 7.29(dd, J=16.4, 11.3Hz, 1H) | (CD3OD, 125MHz)δppm: 19.4(q), 37.9(t), 45.8(t), |
| #57.4(q), 70.1(d), 71.0(d), 82.0(d), 103.6(d), 114.4(s), 116.2(s), 131.7(d), 132.5(d), 135.1(s), 140.8(d), 142.5(d), 157.0(s), 157.2(s), 168.1(s), 200.2(s) | | | |
| 68 | 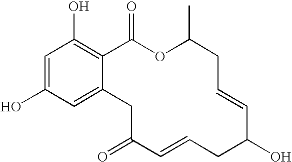 | (Acetone-d6, 500MHz)δppm: 1.28(d, J=6.7Hz, 3H), 2.24(ddd, J=12.8, 7.3, 4.3Hz, 1H), 2.34(dt, J=14.0, 8.5Hz, 1H), 2.48(ddd, J=14.0, 7.3, 4.9Hz, 1H), 2.65(ddd, J=14.0, 7.3, 4.3Hz, 1H), 3.98(d, J=16.5Hz, 1H), 4.04(d, J=16.5Hz, 1H), 4.41(qui, J=4.9Hz, 1H), 5.37(m, 1H), 5.38(m, 1H), 5.52(dt, J=15.3, 7.9Hz, 1H), 5.89(d, J=15.9Hz, 1H), 6.29(d, J=2.4Hz, 1H), 6.31(d, | |
| #J=2.4Hz, 1H), 6.73(ddd, J=15.9, 8.5, 7.3Hz, 1H) | (Acetone-d6, 125MHz)δppm: 18.7(q), 37.2(t), 40.7(t), 48.3(t), 72.1(d), 72.8(d), 102.8(t), 106.6(s), 113.2(d), 125.2(d), 132.5(d), 137.6(d), 141.2(s), 144.9(d), 163.0(s), 165.8(s), 171.0(s), 196.8(s) | | |
| 69 | 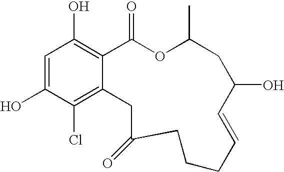 | (CD3OD, 500MHz)δppm: 1.37(d, J=6.7Hz, 3H), 1.65(m, 1H), 1.75(m, 1H), 1.91(m, 1H), 2.00(dd, J=15.3, 7.9Hz, 1H), 2.10(ddd, J=15.3, 7.9, 3.0Hz, 1H), 2.18(m, 1H), 2.30(ddd, J=19.5, 6.7, 3.0Hz, 1H), 2.58(ddd, J=19.5, 10.4, 3.0Hz, 1H), 4.12(d, J=18.3Hz, 1H), 4.23(dt, J=7.9, 3.0Hz, 1H), 4.42(d, J=18.3Hz, 1H), 5.24(dd, J=15.3, 7.9Hz, 1H), 5.45(qui, J=6.7Hz, 1H), | |
| #5.50(ddd, J=15.3, 10.4, 4.3Hz, 1H), 6.45(s, 1H) | (CD3OD, 125MHz)δppm: 20.5(q), 21.0(t), 32.4(t), 40.2(t), 43.1(t), 47.3(t), 47.3(t), 71.2(d), 71.8(d), 104.0(d), 109.0(s), 116.6(s), 133.0(d), 134.4(d), 137.2(s), 159.8(s), 163.4(s), 171.5(s), 208.5(s) | | |
| 70 | 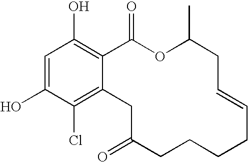 | (Acetone-d, 500MHz)δppm: 1.38(d, J=6.7Hz, 3H), 1.46(m, 1H), 1.59(m, 2H), 1.65(m, 1H), 2.02(m, 1H), 2.10(m, 1H), 2.34(dt, J=14.6, 6.1Hz, 1H), 2.52(m, 1H), 2.56(m, 2H), 4.09(d, J=17.7Hz, 1H), 4.48(d, J=17.7Hz, 1H), 5.34(d-qui, J=6.7, 3.0Hz, 1H), 5.49(d, J=15.3, 5.5Hz, 1H), 5.50(dd, J=15.3, 5.5Hz, 1H), 6.54(s, 1H) | (Acetone-d6, 125MHz)δppm: 19.1(q), 23.0(t), 26.3(t), |
| #32.8(t), 38.3(t), 41.4(t), 46.9(t), 73.8(d), 103.7(d), 108.4(s), 115.9(s), 125.8(d), 135.4(d), 137.9(s), 158.8(s), 163.3(s), 171.0(s), 206.0(s) | | | |
| 71 | 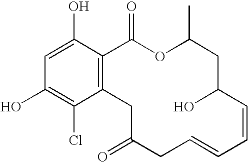 | (CD3OD, 500MHz)δppm: 1.34(d, J=6.1Hz, 3H), 1.56(dt, J=13.4, 1.8Hz, 1H), 2.15(dt, J=12.1, 4.3Hz, 1H), 2.96(dd, J=14.0, 5.5Hz, 1H), 3.27(dd, J=14.0, 6.1Hz, 1H), 3.819d, J=18.3Hz, 1H), 4.65(dt, J=11.0, 4.3Hz, 1H), 4.68(d, J=18.3Hz, 1H), 4.78(ddd, J=12.1, 6.1, 1.8Hz, 1H), 5.26(t, J=11.0Hz, 1H), 5.78(dt, J=15.3, 6.1Hz, 1H), 6.04(dd, J=15.3, 11.0Hz, 1H), 6.15(t, | |
| #J=11.0Hz, 1H), 6.47(s, 1H) | (CD3OD, 125MHz)δppm: 20.6(q), 44.0(t), 44.6(t), 45.2(t), 65.4(d), 71.6(d), 104.0(d), 114.3(s), 115.9(s), 128.4(d), 129.0(d), 130.5(d), 133.9(d), 135.1(s), 157.3(s), 157.7(s), 168.7(s), 206.3(s) | | |
| 72 | 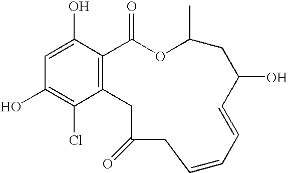 | (CD3OD, 500MHz)δppm: 1.34(d, J=6.1Hz, 3H), 1.73(ddd, J=14.0, 10.0, 3.4Hz, 1H), 2.20(ddd, J=14.0, 11.6, 6.5Hz, 1H), 3.06(dd, J=15.5, 8.2Hz, 1H), 3.29(ddd, J=15.5, 6.7, 1.8Hz, 1H), 3.91(d, J=18.3Hz, 1H), 4.14(sxt, J=5.5Hz, 1H), 4.48(d, J=18.3Hz, 1H), 5.03(dq, J=6.1, 3.4Hz, 1H), 5.64(m, 1H), 5.65(m, 1H), 6.10(dd, J=14.9, 10.7Hz, 1H), 6.18(t, J=10.7Hz, 1H), 6.47(s, | |
| #1H) | (CD3OD, 125MHz)δppm: 20.6(q), 42.5(t), 42.7(t), 45.0(t), 71.3(d), 71.8(d), 104.1(d), 113.4(s), 116.0(s), 124.9(d), 128.2(d), 132.1(d), 135.8(s), 136.9(d), 157.9(s), 158.5(s), 168.9(s), 208.3(s) | | |
| 73 | 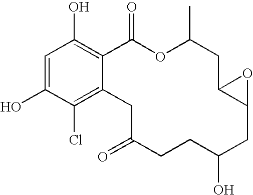 | (CD3OD, 500MHz)δppm: 1.40(d, J=5.7Hz, 3H), 1.43(m, 1H), 1.60(m, 1H), 1.77(m, 1H), 1.80(m, 1H), 1.89(m, 1H), 1.99(m, 1H), 2.10(m, 1H), 2.43(m, 1H), 2.69(m, 1H), 2.83(m, 1H), 3.75(m, 1H), 4.17(d, J=18.3Hz, 1H), 4.25(d, J=18.3Hz, 1H), 5.25(d-qui, J=6.7, 3.0Hz, 1H), 6.42(s, 1H) | (CD3OD, 125MHz)δppm: 19.2(q), 31.5(t), 37.8(t), 38.3(t), 39.6(t), 47.5(t), 55.2(d), 56.7(d), 68.2(d), |
| #72.6(d), 104.0(d), 110.0(s), 116.1(s), 136.5(s), 159.2(s), 162.4(s), 170.8(s), 209.2(s) | | | |
| 74 | 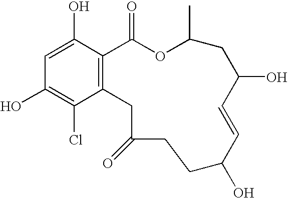 | (CD3OD, 500MHz)δppm: 1.36(d, J=6.7Hz, 3H), 1.75-1.85(m, 2H), 2.03(ddd, J=15.3, 7.3, 1.8Hz, 1H), 2.10(ddd, J=15.9, 7.9, 3.0Hz, 1H), 2.29(ddd, J=19.5, 6.7, 3.0Hz, 1H), 2.54(ddd, J=19.5, 9.8, 3.0Hz, 1H), 3.92(sxt, J=4.9Hz, 1H), 4.08(d, J=17.7Hz, 1H), 4.27(dt, J=9.8, 2.4Hz, 1H), | |
| #4.37(d, J=17.7Hz, 1H), 5.33(dd, J=15.3, 8.5Hz, 1H), 5.43(dd, J=15.3, 8.5Hz, 1H), 5.49(qui, J=6.7Hz, 1H), 6.47(s, 1H) | (CD3OD, 125MHz)δppm: 20.6(q), 29.3(t), 38.0(t), 43.2(t), 47.4(t), 70.9(d), 71.7(d), 73.7(d), 104.1(d), 108.1(s), 116.6(s), 134.6(d), 135.1(d), 137.3(s), 159.9(s), 163.9(s), 171.6(s), 207.7(s) | | |
| 75 | 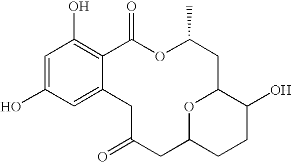 | (CD3OD, 500MHz)δppm: 1.34(d, J=6.7Hz, 3H), 1.58(ddd, J=15.5, 7.3, 4.3Hz, 1H), 1.74(m, 1H), 1.91(m, 1H), 1.94(m, 1H), 2.05(m, 1H), 2.18(dt, J=15.5, 2.4Hz, 1H), 2.40(dd, J=15.5, 4.0Hz, 1H), 2.65(dd, J=15.5, 10.0Hz, 1H), 3.42(ddd, J=10.0, 7.3, 2.4Hz, 1H), 3.58(m, 1H), 3.85(d, J=18.3Hz, 1H), 4.27(d, J=18.3Hz, 1H), 4.38(ddd, J=13.4, 6.7, 3.0Hz, 1H), | |
| #5.32(m, 1H), 5.98(d, J=2.4Hz, 1H), 6.14(d, J=2.4Hz, 1H) | (CD3OD, 125MHz)δppm: 19.0(q), 29.2(t), 31.5(t), 41.1(t), 47.9(t), 52.7(t), 72.9(d), 73.0(d), 77.2(d), 83.0(d), 102.9(d), 108.2(s), 113.0(d), 139.2(s), 163.2(s), 165.1(s), 171.8(s), 208.6(s) | | |
| 76 | 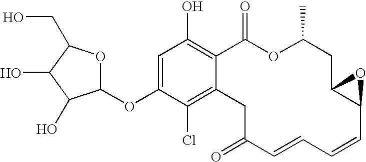 | (CD3OD, 500MHz)δppm: 1.52(d, J=6.1Hz, 3H), 1.65(ddd, J=14.6, 8.5, 3.7Hz, 1H), 2.43(dt, J=14.6, 3.7Hz, 1H), 3.06(dt, J=8.5, 2.4Hz, 1H), 3.35(m, 1H), 3.64(dd, J=12.2, 3.7Hz, 1H), 3.68(dd, J=12.2, 3.7Hz, 1H), 3.92(d, J=16.5Hz, 1H), 4.01(d, J=16.5Hz, 1H), 4.08(dd, J=6.7, 3.0Hz, 1H), 4.17(dd, J=6.7, 3.0Hz, 1H), 4.22(dd, J=6.7, 4.9Hz, 1H), | |
| #5.38(m, 1H), 5.64(d, J=4.9Hz, 1H), 5.75(dd, J=10.4, 4.3Hz, 1H), 6.06(d, J=15.9Hz, 1H), 6.20(t, J=10.4Hz, 1H), 6.85(s, 1H), 7.57(dd, J=15.9, 10.4Hz, 1H) | (CD3OD, 125MHz)δppm: 18.7(q), 37.9(t), 46.3(t), 56.5(d), 56.8(d), 63.1(t), 71.0(d), 72.3(d), 73.5(d), 88.4(d), 103.3(d), 105.3(d), 117.1(s), 117.2(s), 130.9(d), 131.7(d), 134.4(s), 137.2(d), 140.7(d), | | |
| #156.6(s), 157.8(s), 168.4(s), 199.1(s) | | | |
| 77 | 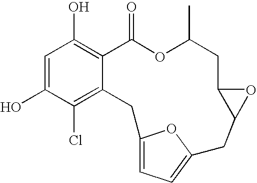 | (Acetone-d6, 500MHz)δppm: 1.38(ddd, J=14.0, 9.2, 3.7Hz, 1H), 1.40(d, J=6.1Hz, 1H), 2.13(dd, J=15.3, 7.9Hz, 1H), 2.38(ddd, J=14.0, 11.0, 4.3Hz, 1H), 2.75(ddd, J=8.5, 3.7, 1.8Hz, 1H), 2.94(ddd, J=8.5, 3.7, 2.4Hz, 1H), 3.24(dd, J=15.3, 3.7Hz, 1H), 4.24(d, J=15.3Hz, 1H), 4.58(d, J=15.3Hz, 1H), 5.15(d-sxt, J=6.1, 3.7Hz, 1H), 5.50(br.d, J=2.4Hz, | |
| #1H), 6.17(br.d, J=2.4Hz, 1H), 6.52(s, 1H) | (Acetone-d6, 125MHz)δppm: 20.5(q), 28.6(t), 32.1(t), 38.3(t), 54.9(d), 56.5(d), 72.3(d), 103.6(d), 107.3(d), 108.3(d), 113.7(s), 114.7(s), 137.6(s), 150.4(s), 141.7(s), 156.4(s), 156.9(s), 168.3(s) | | |
| 78 | 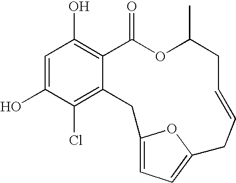 | (Acetone-d6, 500MHz)δppm: 1.38(d, J=6.1Hz, 3H), 2.25(dt, J=13.7, 10.7Hz, 1H), 2.39(dd, J=13.7, 4.6Hz, 1H), 3.18(d, J=7.3Hz, 2H), 4.17(d, J=14.3Hz, 1H), 4.58(d, J=14.3Hz, 1H), 5.40(m, 1H), 5.42(d-qui, J=6.1, 1.8Hz, 1H), 5.61(ddd, J=15.2, 10.4, 4.6Hz, 1H), 5.86(d, J=3.0Hz, 1H), 6.08(d, J=3.0Hz, 1H), 6.47(s, 1H) | (Acetone-d6, 125MHz)δppm: |
| #21.1(q), 27.9(t), 31.4(t), 39.9(t), 71.7(d), 103.2(d), 106.4(d), 107.1(d), 113.5(s), 114.4(s), 128.3(d), 131.7(d), 138.8(s), 151.7(s), 151.9(s), 156.2(s), 156.7(s), 167.0(s) | | | |

### TEST EXAMPLE 1

Expression of WNT-5A mRNA in Dermal Papilla Cells:

Human dermal papilla cells, which were purchased from Toyobo, were cultured in MEM (Invitrogen) containing 12% FBS. Human hair follicle cells were separated from plucked hairs in accordance with the method of Arase et al. (J. Dermatol. Sci., 2, 66-70 (1991)) and cultured by using KGM-2 (Sanko Junyaku).

The dermal papilla cells in passage 5 and the hair follicle cells in passage 2 were sowed in a 10 cm dish to give a density of 2×10⁶ cells/well and cultured over 2 nights. After removing the medium, the cells were washed with PBS(−) and total RNAs were extracted by using TRIzol reagent (Invitrogen). Using 50 ng of the total RNAs, 0.4 μM primers specific to WNT-5A or glyceraldehyde3-phosphate dehydrogenase (GAPDH) [NT-5A forward: AATGTCTTCC AAGTTCTTCC TAGTGGC (SEQ ID NO:8), WNT-5A reverse: GATGTCGGAA TTGATACTGG CA (SEQ ID NO:9), GAPDH forward: ACCACAGTCC ATGCCATCAC (SEQ ID NO:10) and GAPDH reverse: TCCACCACCC TGTTGCTGTA (SEQ ID NO:11)] and SUPERSCRIPT One-Step RT-PCT with PLATINUM Taq (Invitrogen), RT-PCR was carried out in accordance with the protocol attached to SUPERSCRIPT One-Step RT-PCT with PLATINUM Taq. In brief first strand synthesis was carried out at 50° C. for 30 minutes in a reaction system of 25 μl in total volume. After heating to 94° C. for 2 minutes, each cDNA fragment was amplified in 23 or 20 cycles with each cycle consisting of 30 seconds at 94° C., 30 seconds at 55° C. and 30 seconds at 72° C.

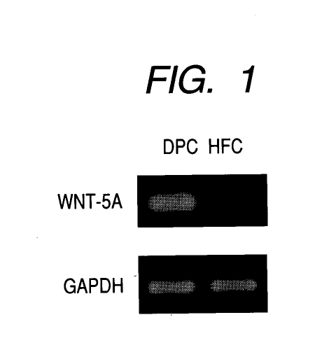

Each reaction solution was electrophoresed in a 1.5% agarose gel and stained with ethidium bromide. FIG. 1 shows the results.

In the case of using the dermal papilla cell (DPC)-derived RNA, a remarkable amplification of the cDNA fragment was observed in the 23-cycle PCR using the WNT-5A-specific primers. In the case of using the hair follicle cell (HFC)-derived RNA, no amplified product was observed under the same conditions.

### TEST EXAMPLE 2

Measurement of Activity of Decreasing WNT-5A mRNA Amount:

Human dermal papilla cells, which were purchased from Toyobo, were cultured in MEM (Invitrogen) containing 12% FBS.

The dermal papilla cells in passage 5 were sowed in a 12-well plate to give a density of 1.6×10⁵ cells/well and cultured overnight. Then, the medium was replaced by a medium to which no compound was added or a medium containing Compound 1, 2, 3, 4, 5, 7, 9 or 24 and the culture was continued for additional 24 hours. After the completion of the culture, each medium was removed and the cells were washed with PBS(−). Ther [symbol] tal RNAs were extracted by using TRIzol reagent (Invitrogen). Using 50 ng of the total RNAs, 0.4 μM of primers specific to WNT-5A or GAPDH [WNT-5A forward; AATGTCTTCC AAGTTCTTCC TAGTGGC (SEQ ID NO:8), WNT-5A reverse: GATGTCGGAA TTGATACTGG CA (SEQ ID NO:9), GAPDH forward: ACCACAGTCC ATGCCATCAC (SEQ ID NO:10) and GAPDH reverse; TCCACCACCC TGTTGCTGTA (SEQ ID NO:11)] and SUPERSCRIPT One-Step RT-PCT with PLATINUM Taq (Invitrogen), RT-PCR was carried out in accordance with the protocol attached to SUPERSCRIPT One-Step RT-PCT with PLATINUM Taq. In brief, first strand synthesis was carried out at 50° C. for 30 minutes in a reaction system of 25 μl in total volume. After heating to 94° C. for 2 minutes, the cDNA fragment of WNT-5A or GAPDH was amplified in 23 or 20 cycles with each cycle consisting of 30 seconds at 94° C., 30 seconds at 55° C. and 30 seconds at 72° C.

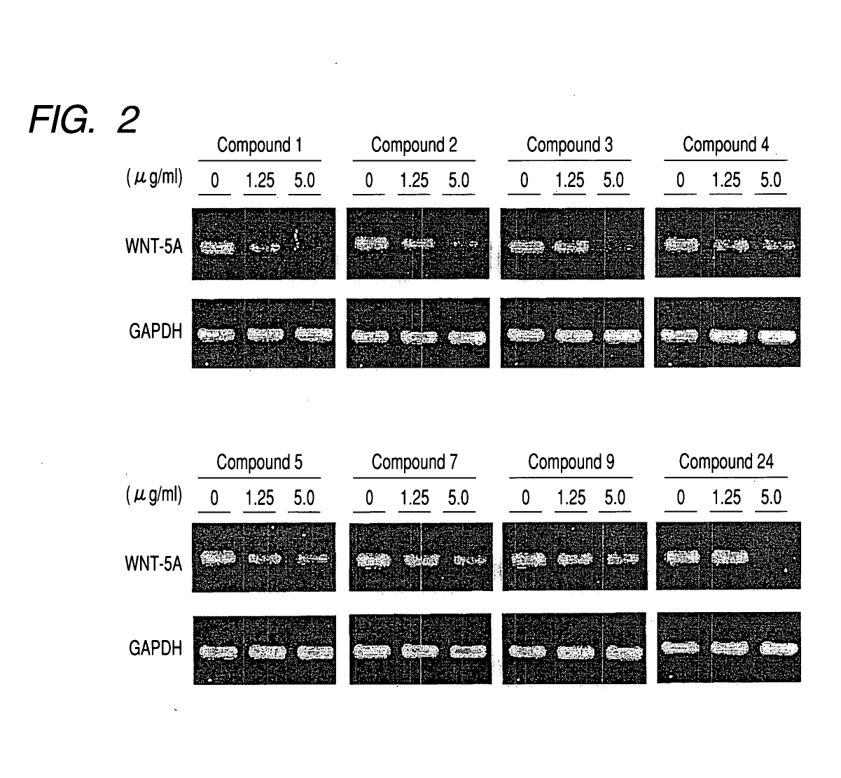

Each reaction solution was electrophoresed in a 1.5% agarose gel and stained with ethidium bromide. FIG. 2 shows the results.

In the 23-cycle PCR using the WNT-5A-specific primers, the amplified WNT-5A cDNA fragments was remarkably decreased in the cultures with the compounds, compared with the culture using the medium to which no compound was added.

In the 20-cycle PCR using the GAPDH-specific primers, on the other hand, no change was observed in the amount of amplified cDNA fragment regardless of the presence or absence of the compounds.

### TEST EXAMPLE 3

Dermal Papilla Cell Growth Promotion Activity Test:

Human dermal papilla cells, which were purchased from Toyobo, were cultured in MEM (Invitrogen) containing 12% FBS.

The dermal papilla cells in passage 5 were sowed in a 96-well plate for spheroid culture to give a density of 1.5×10⁴ cells/well and cultured overnight. Then, the medium was replaced by a medium to which no compound was added or a medium containing Compound 1, 2, 3, 4, 5, 7, 9 or 24 and the culture was continued for additional 72 hours. After the completion of the culture, cells were counted by using Cell Counting Kit (Wako Pure Chemicals). Five hours before the completion of the culture, WST-1 reagent at 1/10 amount per medium was added to each medium and then the optical density (0.1). 450 nm/620 nm) of the medium was measured at the completion of the culture, A positive correlation was observed between the cell number and optical density within the range of 0.25 to 4×10⁴ cells/well.

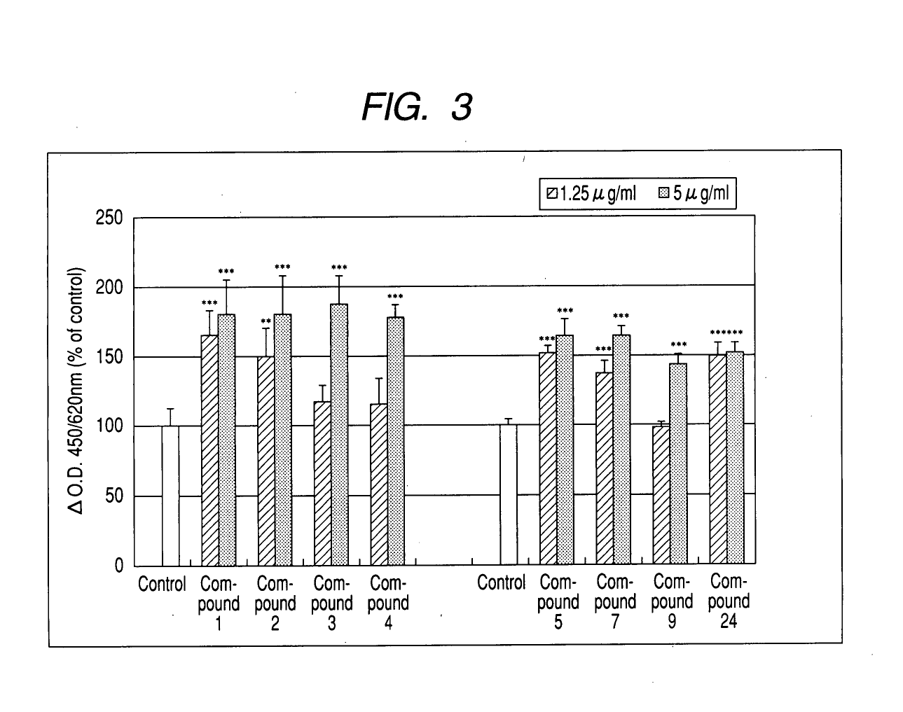

As a result, it is clarified that a compound having an activity of decreasing WNT-5A mRNA amount has an activity of promoting the growth of dermal papilla cells (FIG. 3). In this figure, each numerical value means an average of 6 wells (control group) or 3 wells (test groups). To compare the control group to the test groups, Student's t-test was employed:
*:P<0.05, **:P<0.01, ***:P<0.001.

The above-described Test Examples indicates that WNT-5A is expressed in human dermal papilla cells, the amount of WNT-5A mRNA in dermal papilla cells is decreased by the compound according to the present invention, and a compound having an activity of decreasing WNT-5A mRNA amount has an activity of promoting the growth of dermal papilla cells.

### TEST EXAMPLE 4

Activity of Compounds (Table 2) of Decreasing WNT-5A mRNA Amount (Quantification of mRNA by QuantiGene Method)

Human dermal papilla cells, which were purchased from Toyobo, were cultured in MEM (Invitrogen) containing 12% FBS.

The dermal papilla cells in passage 5 were sowed in a 96-well plate to give a density of 1×10⁴ cells/well and cultured overnight. Then, the medium was replaced by a medium to which no compound was added or a medium containing the compounds and the culture was continued for additional 24 hours. After the completion of the culture, WNT-5A or GAPDH mRNA amount was measured by using QuantiGene High Volume Kit (Bayer Medical) by the branched DNA (bDNA) signal amplification method (Drug Metabolism and Disposition, 28(5), 608-616 (2000)). In accordance with the protocol attached to QuantiGene High Volume Kit, cells were lysed by using Lysis Mixture and the lysis solution was added to capture plate. Next, a set of probes specific to WNT-5A or GPDH was added and allowed to react at 53° C. for 20 hours. After washing the plate by using 0.1×SSC containing 0.03% lauryl sulfate, an amplification probe comprising bDNA was added and allowed to react at 46° C. for 1 hour. After washing the plate, a labeling probe labeled with alkaline phosphatase was added and allowed to react at 46° C. for 1 hour. After washing the plate, a substrate Lumi-Phos Plus was added and allowed to react at 46° C. for 30 minutes. Then, the luminescence was measured by using WALLAC 1420ARVOₛₓ.

The WNT-5A-specific probe set was designed based on the sequence of the coding region of human WNT-5A mRNA. As capture extenders (CEs), 10 probes (SEQ ID NOs:12 to 21) were used; as label extenders (LEs), 31 probes (SEQ ID NOs:22 to 52) were employed; and as blockers, 9 probes (SEQ ID NOs:53 to 60) were employed.

As GAPDH-specific probe set, use was made of bDNA probe set for human GAPDH (XenoTech LLC, B0960).

The amounts of WNT-5A and GAPDH mRNAs are expressed in relative values (%) to the control case of adding of no compound and the concentration of a compound at which the mRNA amount is halved (IC₅₀) is calculated as an index of WNT-5A mRNA decreasing activity of the corresponding compound.

Table 3 shows the effects of the compounds on the WNT-5A and GDPH mRNA amounts. Bach numerical value in the table shows the average of 2 wells. These compounds decreased the WNT-5A mRNA amount in dermal papilla cells. The IC₅₀ value of the most active compound was 0.12 μM. At the IC₅₀ concentration of each compound for WNT-5A mRNA suppression, no decrease was observed in the GAPDH mRNA amount measured at the same time.

TABLE 3

IC₅₀ (μM)

| Compound No. | WNT-5A | GAPDH |
|---|---|---|
| 1 | 0.20 | 28.25 |
| 2 | 0.39 | 26.41 |
| 3 | 3.16 | 57.95 |
| 4 | 3.46 | >30 |
| 5 | 3.47 | >30 |
| 7 | 4.07 | >120 |
| 18 | 0.63 | 6.58 |
| 24 | 4.74 | 21.07 |
| 35 | 0.46 | 4.29 |
| 38 | 6.42 | >120 |
| 52 | 0.12 | 13.47 |
| 53 | 0.12 | 45.45 |
| 54 | 2.37 | >30 |
| 55 | 0.74 | >30 |
| 58 | 0.50 | 79.95 |
| 65 | 8.28 | 109.16 |
| 68 | 10.69 | 50.63 |
| 73 | 9.37 | >120 |
| 76 | 8.56 | 55.94 |
| 77 | 8.13 | >120 |
| 79 | 0.90 | 38.16 |

### TEST EXAMPLE 5

Dermal papilla cell growth promoting activity of compounds (Table 2):

Human dermal papilla cells, which were purchased from Toyobo, were cultured in MEM (Invitrogen) containing 12% FBS.

The dermal papilla cells in passage 5 were sowed in a 96-well plate for spheroid culture to give a density of 1.5×10⁴ cells/well and cultured overnight. Then, the medium was replaced by a medium to which no compound was added or a medium containing the compound and the culture was continued for additional 72 hours. After the completion of the culture, cells were counted by using a Cell Counting Kit (Wako Pure Chemicals). Five hours before the completion of the culture, WST-1 reagent at 1/10 amount per medium was added to each medium and then the optical density (O.D. 450 nm/620 nm) of the medium was measured at the completion of the culture. A positive correlation was observed between the cell number and optical density within the range of 0.25 to 4×10⁴ cells/well.

Table 4 shows the effects of the compounds having an activity of decreasing the WNT-5A mRNA amount on the growth of dermal papilla cells. In this table, each numerical value means an average of 6 wells (control group) or 3 wells (test groups). To compare the control group to the test groups, Student's t-test was employed:
*:P<0.05, **:P<0.01, ***:P<0.001.

Bach of the compounds having an activity of decreasing the WNT-5A mRNA amount showed a remarkable activity of promoting the growth of dermal papilla cells at 16 μM. Compounds 1, 2, 18, 35, 52, 53 and 58, each showing a potent activity of decreasing the WNT-5A mRNA amount (i.e., IC₅₀ being lower than 1 μM, also showed a significant growth promoting activity at 1 μM.

TABLE 4

| | ΔO.D. 450 nm/620 nm (% of control) | | |
|---|---|---|---|
| Compound No. | 1 | 4 | 16 (μM) |
| 1 | 132.9 ± 11.6*** | 183.7 ± 12.1*** | 144.7 ± 13.0*** |
| 2 | 142.6 ± 10.1*** | 167.3 ± 8.5*** | 241.0 ± 25.3*** |
| 3 | 92.9 ± 1.6 | 92.9 ± 9.9 | 244.2 ± 33.4*** |
| 4 | 97.4 ± 24.4 | 148.2 ± 4.9*** | 180.2 ± 6.9*** |
| 5 | 110.2 ± 45.0 | 174.7 ± 29.2*** | 236.4 ± 26.4*** |
| 7 | 82.6 ± 3.9 | 169.7 ± 10.4*** | 194.8 ± 12.7*** |
| 18 | 115.6 ± 9.0* | 147.1 ± 11.3*** | 210.9 ± 25.9*** |
| 24 | 108.2 ± 6.9 | 104.3 ± 10.5 | 119.7 ± 3.7** |
| 35 | 160.8 ± 3.5*** | 181.6 ± 3.7*** | 158.6 ± 18.3*** |
| 38 | 103.5 ± 2.6 | 89.6 ± 7.3 | 124.9 ± 5.1** |
| 52 | 124.5 ± 1.0*** | 143.2 ± 9.5*** | 197.0 ± 5.4*** |
| 53 | 126.6 ± 14.3** | 149.5 ± 12.8*** | 184.4 ± 3.6*** |
| 54 | 80.0 ± 2.4 | 97.1 ± 5.6 | 135.0 ± 0.9*** |
| 55 | 89.0 ± 1.8 | 126.9 ± 7.2*** | 175.6 ± 9.8*** |
| 58 | 123.3 ± 1.8*** | 150.4 ± 9.4*** | 217.4 ± 14.5*** |
| 65 | 92.1 ± 6.7 | 87.6 ± 8.7 | 141.0 ± 5.3*** |
| 73 | 91.7 ± 5.7 | 81.2 ± 7.8 | 205.6 ± 9.2*** |
| 76 | 80.0 ± 5.6 | 77.1 ± 7.0 | 120.7 ± 6.3* |
| 77 | 81.6 ± 4.4 | 86.3 ± 5.0 | 180.8 ± 21.7*** |
| 79 | 87.5 ± 9.9 | 98.6 ± 3.6 | 120.0 ± 4.1** |

*p < 0.05,
**p < 0.01,
***p < 0.001

### TEST EXAMPLE 6

Decrease in WNT-5A mRNA in Monkey Skin Organ Culture

The dorsal skin of a Cynomolgus monkey (Macaca fascicularis) (male, 6 years old) was collected and divided into pieces (5 mm×8 mm) under a stereoscopic microscope. Then each skin tissue piece was cultured in William's Medium E (Invitrogen) to which no compound was added or Compound 7 or Compound 3 was added and which contained 10 μg/ml insulin (Sigma) and 10 ng/ml hydrocortisone (Kurabo).

On the 11th day of the culturing, hair follicles were separated from each skin tissue piece and total RNAs were extracted by using TRIzol reagent (Invitrogen). Using 200 ng portions of the total RNAs, 0.4 μM portions of primers specific to WNT-5A or glyceraldehyde3-phosphate dehydrogenase (GAPDH) [WNT-5A sense: AATGTCTTCC AAGTTCTTCC TAGTGGC (SEQ ID NO-8), WNT-5A antisense: GATGTCGGAA TTGATACTGG CA (SEQ ID NO:9), GAPDH sense: ACCACAGTCC ATGCCATCAC (SEQ ID NO:10) and GAPDH antisense: TCCACCACCC TGTTGCTGTA (SEQ ID NO:11)] and SUPERSCRIPT One-Step RT-PCT with PLATINUM Taq (Invitrogen), RT-PCR was cared out in accordance with the protocol attached to SUPERSCRIPT One-Step RT-PCT with PLATINUM Taq. In brief, first strand synthesis was carried out at 50° C. for 30 minutes in a reaction system of 25 μl in total volume in accordance with the protocol attached to SUPERSCRIPT One-Step RT-PCT with PLATINUM Taq. After heating to 94° C. for 2 minutes, the WNT-5A or GPDH cDNA fragment was amplified in 40 or 30 cycles with each cycle consisting of 30 seconds at 94° C., 30 seconds at 55° C. and 30 seconds at 72° C.

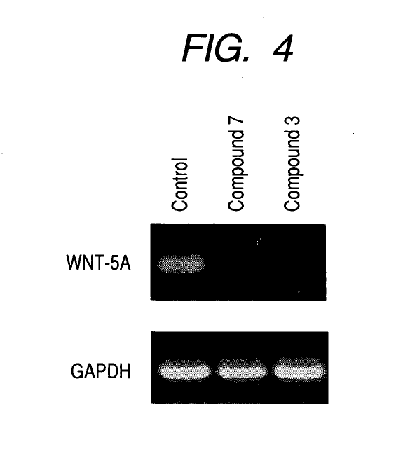

Each reaction solution was electrophoresed in a 1.5% agarose gel and stained with Cyber Green (Takara). FIG. 4 shows the results.

In the 40-cycle PCR using the WNT-5A-specific primers, the amplified WNT-5A cDNA fragment was remarkably decreased in the culture with the addition of Compound 7 or Compound 3, compared with the culture to which no compound was added.

In the 30-cycle PCR using the GAPDH-specific primers, on the other band, no change was observed in the amount of amplified cDNA fragment regardless of the presence or absence of the compounds.

### TEST EXAMPLE 7

Increase in Proliferating Cell Nuclear Antigen (PCNA)-Positive Cells in Monkey Skin Organ Culture:

The dorsal skin of a Cynomolgus monkey (Macaca fascicularis) (male, 6 years old) was collected and divided into pieces (5 mm×8 mm) under a stereoscopic microscope. Then each skin tissue piece was cultured in William's Medium E (Invitrogen) to which no compound was added or Compound 7 or Compound 3 was added and which contained 10 μg/ml of insulin (Sigma) and 10 ng/ml of hydrocortisone (Kurabo).

On the 30th day of the culturing, the skin tissue pieces were each fixed by using 10% neutralized Formalin at room temperature. A section embedded in paraffin was prepared. After adding 1% BSA-containing PBS, it allowed to stand at room temperature for 30 minutes and then boiled in a 0.1 M citrate buffer solution. Then it was reacted with anti-PCNA antibody (Dako Japan, diluted 200-fold) at 4° C. overnight and then reacted with biotinylated antimouse IgG antibody. Further, it was reacted with ABC standard kit (vector) and subjected to color development using AEC substance (Sigma).

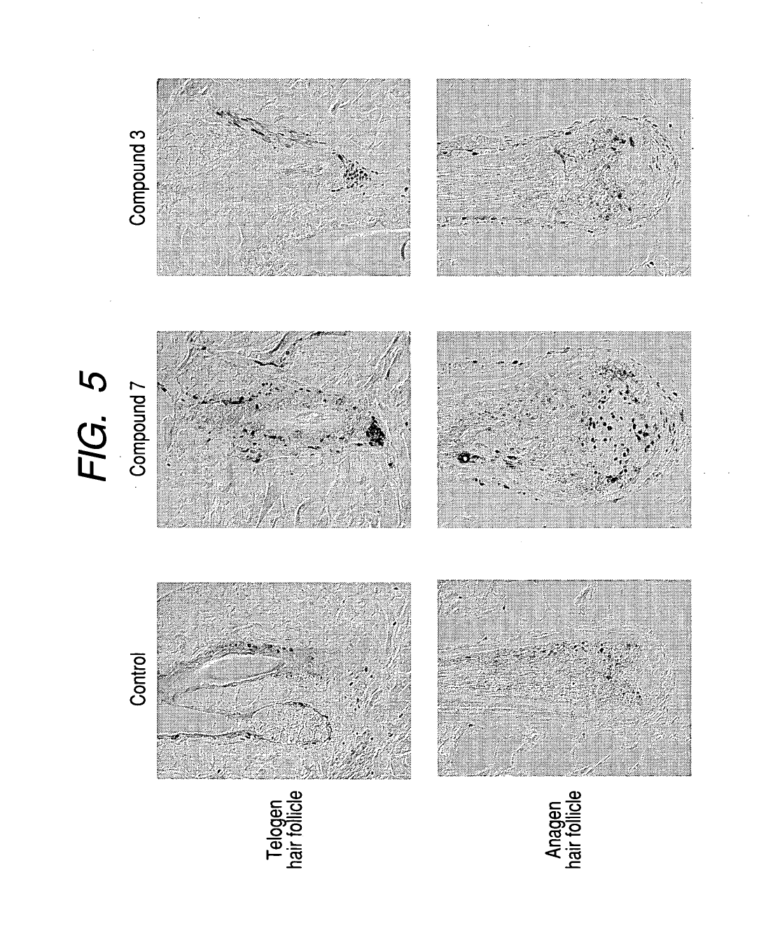

As a result, a positive reaction was observed in cells in hair follicles and the epidermal basal layer. In follicles, remarkable increases in the PCNA-positive cell count are observed in dermal papillae and dermal sheath both in the telogen and anagen hair follicles (FIG. 5).

### TEST EXAMPLE 8

Enlargement in Hair Bulb Diameter in Monkey Skin Organ Culture:

The dorsal skin of a Cynomolgus monkey (Macaca fascicularis) (male, 6 years old) was collected and divided into pieces (5 mm×8 mm) under a stereoscopic microscope. Then each skin tissue piece was cultured in William's Medium E (Invitrogen) to which no compound was added or Compound 7 or Compound 3 was added and which contained 10 μg/ml of insulin (Sigma) and 10 ng/ml of hydrocortisone (Kurabo).

On the 30th day of the culturing, all follicles contained in each skin tissue piece were separated and the diameter of bulbs was measured under a stereoscopic microscope.

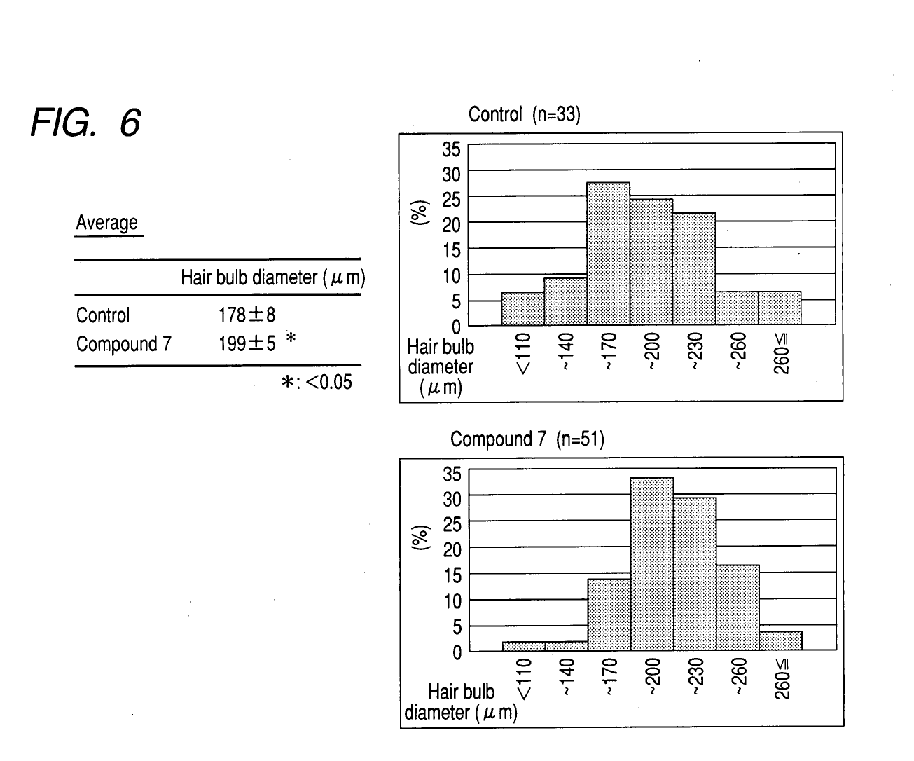

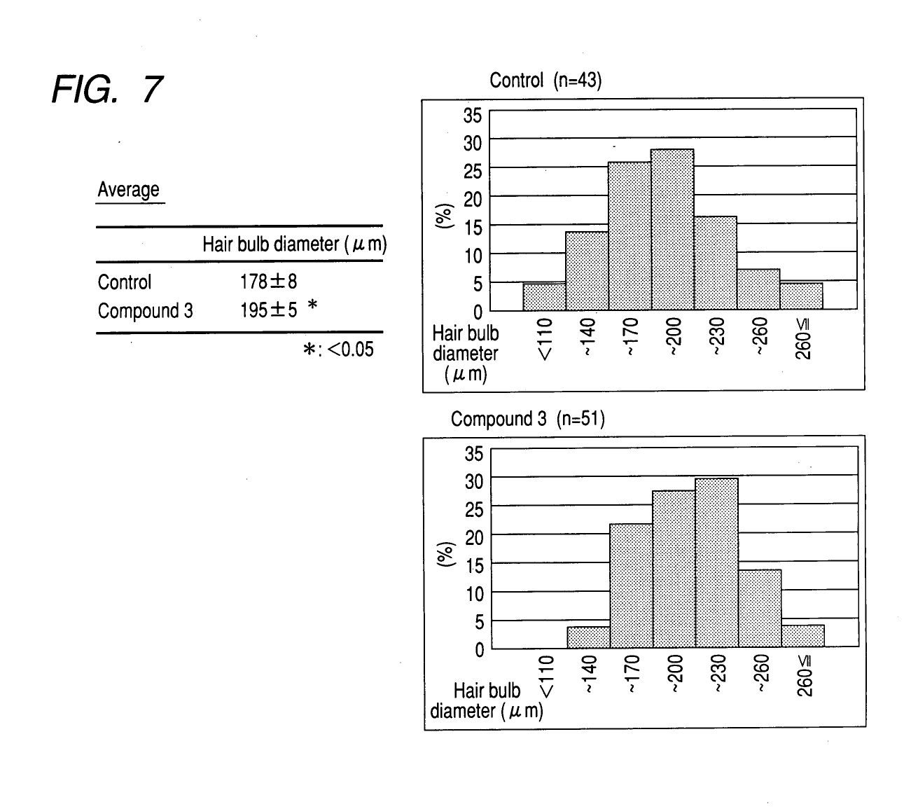

FIGS. 6 and 7 show the results. In these figures, each numerical value means mean±S.E. of bulb diameters of all follicles contained in each skin tissue piece. To compare the control group with the test groups, Student's t-test was employed:
*:P<0.05.

These figures also present histograms showing the bulb diameter distribution. All follicles contained in each skin tissue piece are divided into bulb diameter groups as indicated in the figures and follicle count in each group is expressed in the relative value (%) to the total follicle count separated from the tissue piece. FIG. 6 shows the effect achieved after culturing in the presence of Compound 7. After culturing in the presence of Compound 7 for 30 days, the average bulb diameter of the follicles contained in the skin piece was significantly higher than that of the control group to which no compound was added. As shown by the distribution, the control group to which no compound was added showed the largest follicle count in the group of diameter of 140 μm or longer but shorter than 170 μm, whereas the group to which Compound 7 was added showed the largest follicle count in the group of diameter of 170 μm or longer but shorter than 200 μm. The ratio of follicles of 170 μm or longer in bulb diameter in the group to which no compound was added was 57.6%, whereas the ratio in the group to which Compound 7 was elevated to 82.4%.

On the other hand, FIG. 7 shows the effect achieved after culturing in the presence of Compound 3. Similar to the case of Compound 7, after culturing in the presence of Compound 3 for 30 days, the average bulb diameter of the follicles contained in the skin piece was significantly higher than that of the control group to which no compound was added. As shown by the distribution, the control group to which no compound was added showed the largest follicle count in the group of diameter of 140 μm or longer but shorter than 170 μm, whereas the group to which Compound 3 was added showed the largest follicle count in the group of diameter of 200 μm or longer but shorter than 230 μm. The ratio of follicles of 170 μm or longer in bulb diameter in the group to which no compound was added was 55.8%, whereas the ratio in the group to which Compound 7 was elevated to 74.5%.

### TEST EXAMPLE 9

Hair Growth te [symbol] Stumptailed Macaque:

To the forehead of a stumptailed macaque (Macaca arctoides) (male, 14 years old) suffering from alopecia, 1 mL of a lotion containing 0.3% Compound 7 was applied once a day 5 times per week for 6 months. Before the initiation of the application, the alopecia site in the forehead was marked by tattoo. The skin part including the tattoo was photographed before the initiation of the application and after applying for 6 months.

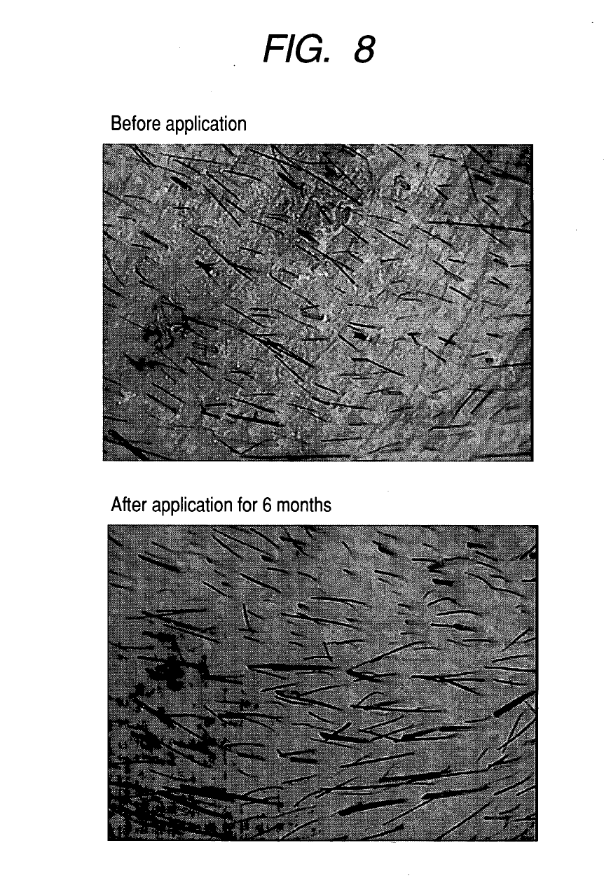

FIG. 8 provides enlarged photos of the skin before and after application of the lotion containing Compound 7 for 6 months. Before the application, many thin vellus hairs were observed. In the same site after applying for 6 months in contrast, vellus hairs decreased and thick terminal hairs increased. That is, it is recognized that the alopecia in the forehead of the stumptailed macaque was clearly ameliorated by applying the lotion containing Compound 7 for 6 months.

The results of the above Test Examples indicate that WNT-5A is expressed in human dermal papilla cells, the amount of WNT-5A mRNA in human dermal papilla cells is decreased by the compound according to the present invention, and a compound having an activity of decreasing WNT-5A mRNA amount has an activity of promoting the growth of dermal papilla cells.

It is also indicated that the compound according to the present invention causes a decrease in the WNT-5A mRNA amount in monkey skin organ culture too, the compound causes a remarkable increase in the PCNA-staining positive proliferative cells both in the telogen and anagen hair follicles, and the compound enlarges the bulb size of follicles.

It is furthermore indicated that the compound according to the present invention has an affect of ameliorating alopecia of a stumptailed macaque which is a model animal of male pattern alopecia.

That is to say, a compound promoting the growth of dermal papilla cells would enhance the cell growing ability, which has been lowered in the dermal papillae in follicles in a part with alopecia, and thus contributes to the formation of well-developed dermal papilla tissues, thereby promoting hair growth.

### INDUSTRIAL APPLICABILITY

A WNT-5A function inhibitor, for example, a compound having an activity of decreasing WNT-5A mRNA amount or a pharmaceutically acceptable salt thereof has an activity of promoting the growth of dermal papilla cells and, therefore, is useful as a hair growth stimulant/hair growth tonic with a novel function mechanism.

The present invention provides a completely novel idea of using a compound inhibiting the function of WNT-5A as an agent ameliorating or preventing alopecia, which has never been known hitherto, and screening of such a compound inhibiting the function of WNT-5A is useful in developing a novel hair growth stimulant/air growth tonic.

1. A dermal papilla cell growth promoter containing a compound having an activity of inhibiting the function of WNT-5A.

2. The dermal papilla cell growth promoter according to claim 1 wherein the compound inhibiting the function of WNT-5A is a WNT-5A production inhibitor.

3. A compound represented by the following formula (I): 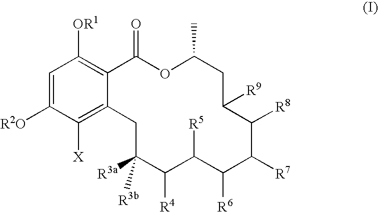
wherein R¹ and R² are the same or different and each represents a hydrogen atom, a C₁₋₆ alkyl group or a C₂₋₆ alkanoyl group;
X represents a hydrogen atom or a halogen atom;
R^{3a} and R^{3b} are the same or different and each represents a hydrogen atom or a hydroxyl group; and
R⁴, R⁵, R⁶, R⁷, R⁸ and R⁹ are the same or different and each represents a hydrogen atom, a hydroxyl group, a halogen atom or a C₂₋₆ alkanoyloxy group, or adjacent groups thereof are taken together to form a π bond or an ether bond, or R⁵ and R⁸ or R⁵ and R⁹ are taken together to form an ether bond.

4. A dermal papilla cell growth promoter containing a compound represented by the following formula (II): 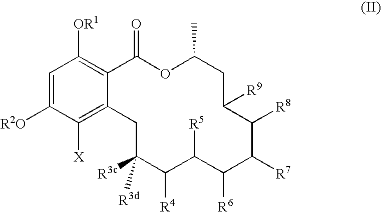
wherein R¹ and R² are the same or different and each represents a hydrogen atom, a C₁₋₆ alkyl group or a C₂₋₆ alkanoyl group; X represents a hydrogen atom or a halogen atom;
R^{3c} and R^{3d} are the same or different and each represents a hydrogen atom, a hydroxyl group or a C₁₋₆ alkoxy group, or R^{3c} and R^{3d} are taken together to form an oxo group, a hydroxyimino group or a C₁₋₆ alkoxyimino group; and
R⁴, R⁵, R⁶, R⁶, R₇, R⁸ and R⁹ are the same or different and each represents a hydrogen atom, a hydroxyl group, a halogen atom or a C₂₋₆ alkanoyloxy group, or adjacent groups thereof are taken together to form a π bond or an ether bond, or R⁵ and R⁸ or R⁵ and R⁹ are taken together to form an ether bond.

5. A dermal papilla cell growth promoter containing a compound represented by the following formula (IX): 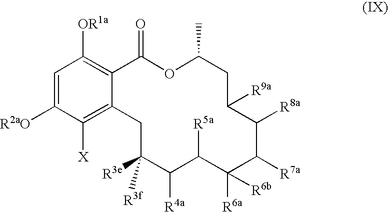
wherein R^{1a} and R^{2a} are the same or different and each represents a hydrogen atom, a C₁₋₆ alkyl group, a C₂₋₆ alkanoyl group,
a group represented by (CH₂)ₚCO—Y—R¹⁰, wherein Y represents an oxygen atom or a sulfur atom; R¹⁰ represents a hydrogen atom, a C₁₋₆ alkyl group or a substituted or unsubstituted aryl group; and p is 0 or 1,
a group represented by (CH₂)_{q}R¹¹, wherein R¹¹ represents a substituted or unsubstituted cycloalkyl group or a substituted or unsubstituted C₂₋₁₀ heterocycle; and q is 0 or 1, or
a group represented by COR¹², wherein R¹² represents a substituted or unsubstituted aryl group or a substituted or unsubstituted C₂₋₁₀ heterocycle;
X represents a hydrogen atom or a halogen atom;
R^{3e} and R^{3f} are the same or different and each represents a hydrogen atom, a hydroxyl group, a C₁₋₆ alkoxy group or a C₁₋₆ alkanoyloxy group, or R^{3e} and R^{3f} are taken together to form an oxo group, a hydroxyimino group or a C₁₋₆ alkoxyimino group; and
R^{4a}, R^{5a}, R^{6a}, R^{7a}, R^{8a} and R^{9a} are the same or different and each represents a hydrogen atom, a hydroxyl group, a halogen atom or
a group represented by Z-R¹³, wherein Z represents an oxygen atom or a sulfur atom; and R¹³ represents a C₁₋₆ alkyl group, a C₁₋₆ alkanoyl group or a substituted or unsubstituted aryl group, or
adjacent groups thereof are taken together to form a π bond or an ether bond, or
R^{5a} and R^{8a} or R^{5a} and R₉ₐ are taken together to form an ether bond; and
R^{6b} represents a hydrogen atom, or is taken together with R^{3e} and R^{3f} to form an ether bond.

6. A hair growth stimulant or a hair growth tonic which comprises the dermal papilla cell growth promoter according to any one of claims 1, 2, 4 and 5 as an active ingredient.

7. A method of stimulating hair growth or promoting hair growth, which comprises administering a pharmaceutically effective amount of a compound having an activity of inhibiting the function of WNT-5A to human.

8. Use of a compound having an activity of inhibiting the function of WNT-5A in producing a hair growth stimulant or a hair growth tonic.

9. A method of screening a dermal papilla cell growth promoter, which comprises selecting a compound inhibiting the function of WNT-5A.

10. The method according to claim 9, which comprises the following steps (a) to (c):
(a) a step of culturing human WNT-5A-expressing cells in a medium to which a compound has been added;
(b) a step of lysing the human WNT-5A-expressing cells cultured in the step (a) to extract RNA, and determining the WNT-5A mRNA amount; and
(c) a step of comparing the WNT-5A mRNA amount determined in the step (b).
